SEQUENCE LISTING (52 sequences)

SEQ ID NO: 1 - protein, 9 aa, Artificial Sequence
  Description of Artificial Sequence Synthetic peptide
HMTEVVRHC

SEQ ID NO: 2 - protein, 10 aa, Artificial Sequence
  Description of Artificial Sequence Synthetic peptide
GMNQRPILTI

SEQ ID NO: 3 - protein, 10 aa, Artificial Sequence
  Description of Artificial Sequence Synthetic peptide
GMNWRPILTI

SEQ ID NO: 4 - protein, 9 aa, Artificial Sequence
  Description of Artificial Sequence Synthetic peptide
MPFATPMEA

SEQ ID NO: 5 - protein, 9 aa, Artificial Sequence
  Description of Artificial Sequence Synthetic peptide
GILGFVFTL

SEQ ID NO: 6 - protein, 9 aa, Artificial Sequence
  Description of Artificial Sequence Synthetic peptide
SLYNTVATL

SEQ ID NO: 7 - protein, 10 aa, Artificial Sequence
  Description of Artificial Sequence Synthetic peptide
ELAGIGILTV

SEQ ID NO: 8 - protein, 9 aa, Artificial Sequence
  Description of Artificial Sequence Synthetic peptide
HMTEVVRRC

SEQ ID NO: 9 - protein, 10 aa, Artificial Sequence
  Description of Artificial Sequence Synthetic peptide
GMNRRPILTI

SEQ ID NO: 10 - protein, 9 aa, Artificial Sequence
  Description of Artificial Sequence Synthetic peptide
EVVRHCPHH

SEQ ID NO: 11 - protein, 10 aa, Artificial Sequence
  Description of Artificial Sequence Synthetic peptide
MTEVVRHCPH

SEQ ID NO: 12 - protein, 9 aa, Artificial Sequence
  Description of Artificial Sequence Synthetic peptide
TEVVRHCPH

SEQ ID NO: 13 - protein, 11 aa, Artificial Sequence
  Description of Artificial Sequence Synthetic peptide
EVVRHCPHHER

SEQ ID NO: 14 - protein, 10 aa, Artificial Sequence
  Description of Artificial Sequence Synthetic peptide
VVRHCPHHER

SEQ ID NO: 15 - protein, 9 aa, Artificial Sequence
  Description of Artificial Sequence Synthetic peptide
VRHCPHHER

SEQ ID NO: 16 - protein, 11 aa, Artificial Sequence
  Description of Artificial Sequence Synthetic peptide
NSSCMGGMNQR

SEQ ID NO: 17 - protein, 9 aa, Artificial Sequence
  Description of Artificial Sequence Synthetic peptide
SCMGGMNQR

SEQ ID NO: 18 - protein, 10 aa, Artificial Sequence
  Description of Artificial Sequence Synthetic peptide
SSCMGGMNQR

SEQ ID NO: 19 - protein, 10 aa, Artificial Sequence
  Description of Artificial Sequence Synthetic peptide
QRPILTIITL

SEQ ID NO: 20 - protein, 10 aa, Artificial Sequence
  Description of Artificial Sequence Synthetic peptide
MGGMNQRPIL

SEQ ID NO: 21 - protein, 9 aa, Artificial Sequence
  Description of Artificial Sequence Synthetic peptide
GGMNQRPIL

SEQ ID NO: 22 - protein, 9 aa, Artificial Sequence
  Description of Artificial Sequence Synthetic peptide
NQRPILTII

SEQ ID NO: 23 - protein, 9 aa, Artificial Sequence
  Description of Artificial Sequence Synthetic peptide
MNQRPILTI

SEQ ID NO: 24 - protein, 10 aa, Artificial Sequence
  Description of Artificial Sequence Synthetic peptide
MNQRPILTII

SEQ ID NO: 25 - protein, 10 aa, Artificial Sequence
  Description of Artificial Sequence Synthetic peptide
CVCACPGRDR

SEQ ID NO: 26 - protein, 10 aa, Artificial Sequence
  Description of Artificial Sequence Synthetic peptide
HVCACPGRDR

SEQ ID NO: 27 - protein, 11 aa, Artificial Sequence
  Description of Artificial Sequence Synthetic peptide
NLLGRNSFEVL

SEQ ID NO: 28 - protein, 10 aa, Artificial Sequence
  Description of Artificial Sequence Synthetic peptide
LLGRNSFEVL

SEQ ID NO: 29 - protein, 9 aa, Artificial Sequence
  Description of Artificial Sequence Synthetic peptide
LGRNSFEVL

SEQ ID NO: 30 - protein, 10 aa, Artificial Sequence
  Description of Artificial Sequence Synthetic peptide
EVCVCACPGR

SEQ ID NO: 31 - protein, 10 aa, Artificial Sequence
  Description of Artificial Sequence Synthetic peptide
EVHVCACPGR

SEQ ID NO: 32 - protein, 9 aa, Artificial Sequence
  Description of Artificial Sequence Synthetic peptide
VLVCACPGR

SEQ ID NO: 33 - protein, 10 aa, Artificial Sequence
  Description of Artificial Sequence Synthetic peptide
EVLVCACPGR

SEQ ID NO: 34 - protein, 9 aa, Artificial Sequence
  Description of Artificial Sequence Synthetic peptide
GRNSFEVCV

SEQ ID NO: 35 - protein, 9 aa, Artificial Sequence
  Description of Artificial Sequence Synthetic peptide
GRNSFEVHV

SEQ ID NO: 36 - protein, 11 aa, Artificial Sequence
  Description of Artificial Sequence Synthetic peptide
LLGRNSFEVHV

SEQ ID NO: 37 - protein, 10 aa, Artificial Sequence
  Description of Artificial Sequence Synthetic peptide
LGRNSFEVLV

SEQ ID NO: 38 - protein, 9 aa, Artificial Sequence
  Description of Artificial Sequence Synthetic peptide
GRNSFEVLV

SEQ ID NO: 39 - protein, 11 aa, Artificial Sequence
  Description of Artificial Sequence Synthetic peptide
LLGRNSFEVLV

SEQ ID NO: 40 - protein, 10 aa, Artificial Sequence
  Description of Artificial Sequence Synthetic peptide
FEVCVCACPG

SEQ ID NO: 41 - protein, 9 aa, Artificial Sequence
  Description of Artificial Sequence Synthetic peptide
FEVCVCACP

SEQ ID NO: 42 - protein, 10 aa, Artificial Sequence
  Description of Artificial Sequence Synthetic peptide
FEVHVCACPG

SEQ ID NO: 43 - protein, 9 aa, Artificial Sequence
  Description of Artificial Sequence Synthetic peptide
FEVHVCACP

SEQ ID NO: 44 - protein, 9 aa, Artificial Sequence
  Description of Artificial Sequence Synthetic peptide
FEVLVCACP

SEQ ID NO: 45 - protein, 10 aa, Artificial Sequence
  Description of Artificial Sequence Synthetic peptide
FEVLVCACPG

SEQ ID NO: 46 - protein, 10 aa, Artificial Sequence
  Description of Artificial Sequence Synthetic peptide
RVCACPGRDW

SEQ ID NO: 47 - protein, 10 aa, Artificial Sequence
  Description of Artificial Sequence Synthetic peptide
WRTEEENLRK

SEQ ID NO: 48 - protein, 9 aa, Artificial Sequence
  Description of Artificial Sequence Synthetic peptide
CACPGRDWR

SEQ ID NO: 49 - protein, 11 aa, Artificial Sequence
  Description of Artificial Sequence Synthetic peptide
RVCACPGRDWR

SEQ ID NO: 50 - protein, 13 aa, Artificial Sequence
  Description of Artificial Sequence Synthetic peptide
GMNWRPILTIITL

SEQ ID NO: 51 - protein, 13 aa, Artificial Sequence
  Description of Artificial Sequence Synthetic peptide
GMNQRPILTIITL

SEQ ID NO: 52 - protein, 13 aa, Artificial Sequence
  Description of Artificial Sequence Synthetic peptide
CMGGMNWRPILTI

(19) United States
(12) Patent Application Publication
(10) Pub. No.: US 2025/0322927 A1
(43) Pub. Date: Oct. 16, 2025

(54) MODELS FOR PREDICTING MUTANT P53 FITNESS AND THEIR IMPLICATIONS IN CANCER THERAPY

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US); Institute for Advanced Study, Princeton, NJ (US); Icahn School of Medicine at Mount Sinai, New York, NY (US)

(72) Inventors: Benjamin GREENBAUM, New York, NY (US); David HOYOS, New York, NY (US); Taha MERGHOUB, New York, NY (US); Jedd WOLCHOK, New York, NY (US); Roberta ZAPPASODI, New York, NY (US); Matthew D. HELLMANN, New York, NY (US); Zachary SETHNA, New York, NY (US); Isabell SCHULZE, New York, NY (US); Vinod BALACHANDRAN, New York, NY (US); Arnold LEVINE, Princeton, NJ (US); Marta LUKSZA, New York, NY (US)

(21) Appl. No.: 18/546,762

(22) PCT Filed: Feb. 16, 2022
(86) PCT No.: PCT/US2022/016594

§ 371 Date: Aug. 16, 2023

Related U.S. Application Data
(60) Provisional application No. 63/150,479, filed on Feb. 17, 2021.

(51) Int. Cl. G16H 20/17; C12Q 1/6886; G16B 15/30; G16B 20/10; G16B 20/20; G16B 20/50

(52) U.S. Cl. CPC G16H 20/17; C12Q 1/6886; G16B 15/30; G16B 20/10; G16B 20/20; G16B 20/50; C12Q 2600/106; C12Q 2600/156

(57) ABSTRACT

The present technology relates to methods, computing devices, and systems for predicting the fitness of mutant p53 based on the loss of transcription factor function and immunogenicity of a particular TP53 mutation. The fitness of mutant p53 may be used to determine whether a patient will benefit from a particular anti-cancer therapy such as immune checkpoint inhibitor therapy, adoptive T-cell therapy, or prophylactic cancer vaccine therapy.

### CROSS-REFERENCE TO RELATED APPLICATIONS

This application claims the benefit of and priority to U.S. Provisional Patent Application No. 63/150,479, filed Feb. 17, 2021, the entire contents of which is incorporated herein by reference.

### SEQUENCE LISTING

The instant application contains a Sequence Listing which has been submitted electronically in ASCII format and is hereby incorporated by reference in its entirety. Said ASCII copy, created on Mar. 21, 2024, is named 115872-2853_SL.txt and is 12,712 bytes in size.

### TECHNICAL FIELD

The present technology relates to methods, devices, and systems for predicting the fitness of mutant p53 based on the loss of transcription factor function and immunogenicity of a particular TP53 mutation. The fitness of mutant p53 may be used to determine whether a patient will benefit from a particular anti-cancer therapy such as immune checkpoint inhibitor therapy, adoptive T-cell therapy, or prophylactic cancer vaccine therapy.

### BACKGROUND

The following description of the background of the present technology is provided simply as an aid in understanding the present technology and is not admitted to describe or constitute prior art to the present technology.

Due to the role of p53 protein as a cell cycle checkpoint for general stress responses, cancer cells gain an enormous selective advantage by having a mutated non-functional p53, generating offspring with poor fidelity. The spectrum of somatic TP53 mutations is highly skewed: while the p53 protein is 393 amino acids long and there are theoretically 2,314 possible missense mutations, only 8 mutations comprise one-third of all the TP53 missense mutations found in tumors. The predominance of these hotspots is highly consistent across databases, populations, and tissues. Several hypotheses have been offered to explain this predominance, including biases in generative mutational processes during tumor evolution, degree of loss of transcription factor function, structural stability, and conservation. Moreover, while TP53 mutations can potentially generate appealing shared tumor-associated neoantigens to target with emerging precision immunotherapies, such as neoantigen-based cancer vaccines, these hotspots are typically predicted to be poor antigens. Mutant p53 proteins are typically present at a far higher concentration than wild-type p53, in a way that is tissue-, copy-number-, and mutation-specific, which could make mutant p53 a better antigen than its wild-type counterpart. However, concentration alone does not predict recognition by T-cells for p53 neoantigens. Determining the extent to which each mechanism specifically drives the skewed distribution of TP53 mutations has significant implications both for exploiting mutant p53 driver genetic mutations, as precision immunotherapy targets and understanding tumor evolution.

Thus, there is a substantial need for methods that are useful in predicting whether individual patients harboring TP53 mutations would benefit from immunotherapeutic interventions.

### SUMMARY OF THE PRESENT TECHNOLOGY

In one aspect, the present disclosure provides a method for selecting a candidate therapy based on mutant p53 fitness, comprising: (a) obtaining or otherwise receiving, by one or more processors of a computing device, a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating, by one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating, by one or more processors, an immunogenic cost metric for the p53 missense mutation based on binding affinities of MHC class I molecules to p53-derived nonamer neopeptides including the p53 missense mutation; and (iii) generating, by one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying, by one or more processors, a subset of p53 missense mutations that have fitness scores that exceed a threshold; (d) selecting adoptive T-cell therapy or neoantigen vaccine therapy for the subset of p53 missense mutations; and (e) storing, by one or more processors, in a computer-readable non-volatile memory device, adoptive T-cell therapy or neoantigen vaccine therapy in association with the subset of p53 missense mutations as a candidate therapy. The neoantigen vaccine therapy may be a RNA neoantigen vaccine, a synthetic long peptide neoantigen vaccine, or a dendritic cell (DC)-based neoantigen vaccine. In some embodiments, the pro-oncogenic advantage metric is assigned a greater weight relative to the immunogenic cost metric. Additionally or alternatively, in some embodiments, the method further comprises administering the adoptive T-cell therapy or neoantigen vaccine therapy to a patient comprising at least one p53 missense mutation that is present in the subset of p53 missense mutations. Additionally or alternatively, in some embodiments of the methods disclosed herein, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric. In any of the above embodiments of the methods disclosed herein, the dataset is generated from DNA sequencing data obtained from one or more patients diagnosed with or at risk for cancer or Li-Fraumeni syndrome (LFS). Examples of cancer include, but are not limited to, colorectal cancer, lung cancer, breast cancer, ovarian cancer, uterine cancer, or thyroid cancer.

Additionally or alternatively, in certain embodiments, the at least one p53 target gene is WAF1, MDM2, BAX, h1433s, AIP1, GADD45, NOXA, or P53R2. In some embodiments, the transactivation levels of the at least one p53 target gene are determined using quantitative transactivation assays in yeast.

In any of the preceding embodiments of the methods disclosed herein, the pro-oncogenic advantage metric is a median probability of the p53 polypeptide encoded by the p53 missense mutation not binding to the promoter region of the at least one p53 target gene. In some embodiments, generating the pro-oncogenic advantage metric comprises applying a cooperative Hill function.

In another aspect, the present disclosure provides a method for selecting a candidate anti-cancer therapy based on mutant p53 fitness, comprising: (a) obtaining or otherwise receiving, by one or more processors of a computing device, a dataset comprising a plurality of p53 missense mutations present in one or more subjects; (b) for each p53 missense mutation in the dataset, applying a model to obtain a fitness score, wherein the model comprises: (i) generating, by one or more processors, an immunogenic cost metric for the p53 missense mutation based on binding affinities of MHC class I molecules to p53-derived nonamer neopeptides including the p53 missense mutation; and (ii) generating, by one or more processors, based on the immunogenic cost metric, a fitness score, wherein generating the fitness score comprises applying a divergence-based statistical analysis to optimize the immunogenic cost metric; (c) identifying, by one or more processors, a subset of p53 missense mutations that have fitness scores that fall below a threshold; (d) selecting, by one or more processors, an immune checkpoint blockade therapy for the subset of p53 missense mutations; and (e) storing, by one or more processors, in a computer-readable non-volatile memory, the immune checkpoint blockade therapy in association with the subset of p53 missense mutations as a candidate anti-cancer therapy. Additionally or alternatively, in some embodiments, the method further comprises administering the immune checkpoint blockade therapy to a cancer patient comprising at least one p53 missense mutation that is present in the subset of p53 missense mutations. Examples of immune checkpoint blockade therapy include, but are not limited to, anti-PD-1 antibodies, anti-PD-L1 antibodies, anti-PD-L2 antibodies, anti-CTLA-4 antibodies, anti-TIM3 antibodies, anti-TIGIT antibodies, anti-VISTA antibodies, anti-B7-H3 antibodies, anti-BTLA antibodies, anti-CD73 antibodies, or anti-LAG-3 antibodies. Additionally or alternatively, in some embodiments of the methods disclosed herein, the model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric. In any of the above embodiments disclosed herein, the dataset is generated from DNA sequencing data obtained from one or more patients diagnosed with or at risk for cancer. Examples of cancer include, but are not limited to, colorectal cancer, lung cancer, breast cancer, ovarian cancer, uterine cancer, or thyroid cancer.

In any and all embodiments of the methods disclosed herein, the divergence-based statistical analysis comprises minimizing divergence scores between observed and predicted frequencies of the p53 missense mutation. In certain embodiments, the divergence scores that are minimized are Kullback-Leibler divergences.

Additionally or alternatively, in some embodiments of the methods disclosed herein, the MHC class I molecules comprise HLA-A alleles, HLA-B alleles, and HLA-C alleles. In some embodiments, generating the immunogenic cost metric comprises determining a geometric mean of probabilities of the p53-derived nonamer neopeptides including the p53 missense mutation binding each allele of the MHC class I molecules. Additionally or alternatively, in some embodiments of the methods disclosed herein, the MHC class I molecules comprise one or more HLA alleles selected from the group consisting of A*02:11, A*26:02, A*68:23, C*07:01, A*02:03, A*02:06, C*12:03, A*68:02, A*24:03, B*15:03, B*15:17, B*57:01, B*58:01, A*31:01, A*33:01, A*68:01, A*11:01, A*30:01, A*32:07, B*08:01, C*03:03, A*02:01, A*02:12, A*02:17, B*39:01, and B*73:01.

In any and all embodiments of the methods disclosed herein, the plurality of p53 missense mutations comprises somatic and/or germline p53 mutations.

In one aspect, the present disclosure provides a method for selecting a patient diagnosed with or at risk for cancer for treatment with an immune checkpoint inhibitor comprising: detecting the presence of a p53 mutation in a biological sample obtained from the patient, wherein the p53 mutation is selected from the group consisting of R248Q, R273H, R248W, R273C, and G245S; and administering to the patient an effective amount of the immune checkpoint inhibitor. The p53 mutation may be a germline or somatic mutation. Examples of cancers, include but are not limited to, colorectal cancer, lung cancer, breast cancer, ovarian cancer, uterine cancer, or thyroid cancer. Additionally or alternatively, in some embodiments, the the p53 mutation is detected via in situ hybridization, polymerase chain reaction (PCR), Next-generation sequencing, Northern blotting, microarray, dot or slot blots, fluorescent in situ hybridization (FISH), electrophoresis, chromatography, or mass spectroscopy. In certain embodiments, the biological sample comprises blood, plasma, serum or tissue.

Examples of immune checkpoint inhibitors include, but are not limited to, an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody.

In another aspect, the present disclosure provides a method for characterizing classifying tumor behavior for a potential tumor based on mutant p53 fitness, comprising: (a) obtaining, by one or more processors of a computing device, a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating, by the one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating, by the one or more processors, an immunogenic cost metric for the p53 missense mutation based on binding affinities of MHC class I molecules to p53-derived nonamer neopeptides including the p53 missense mutation; and (iii) generating, by the one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying, by the one or more processors, a subset of p53 missense mutations that have fitness scores that exceed a threshold; (d) identifying, by the one or more processors, at least one of an age of tumor onset or a tumor type corresponding to the potential tumor for the subset of p53 missense mutations; and (e) storing, by the one or more processors, in a computer-readable non-volatile memory device, the at least one of the age of tumor onset or the tumor type in association with the subset of p53 missense mutations as a tumor behavior classification. Additionally or alternatively, in some embodiments of the methods disclosed herein, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

In any and all embodiments disclosed herein, the tumor behavior classification may identify the age of tumor onset as 10-20 years.

In any and all embodiments disclosed herein, the tumor behavior classification may identify the age of tumor onset as 30-50 years.

In any and all embodiments disclosed herein, the tumor behavior classification may identify the age of tumor onset as 50 years or older.

In any and all embodiments disclosed herein, the pro-oncogenic advantage metric may have a greater weight relative to the immunogenic cost metric.

In any and all embodiments disclosed herein, the at least one p53 target gene may be WAF1, MDM2, BAX, h1433s, AIP1, GADD45, NOXA, or P53R2.

In any and all embodiments disclosed herein, the transactivation levels of the at least one p53 target gene may be determined using quantitative transactivation assays in yeast.

In any and all embodiments disclosed herein, the pro-oncogenic advantage metric may be a median probability of the p53 polypeptide encoded by the p53 missense mutation not binding to the promoter region of the at least one p53 target gene.

In any and all embodiments disclosed herein, generating the pro-oncogenic advantage metric may comprise applying, by the one or more processors, a cooperative Hill function.

In any and all embodiments disclosed herein, the divergence-based statistical analysis may comprise minimizing, by the one or more processors, divergence scores between observed and predicted frequencies of the p53 missense mutation.

In any and all embodiments disclosed herein, the divergence scores that are minimized may be Kullback-Leibler divergences.

In any and all embodiments disclosed herein, the MHC class I molecules may compris HLA-A alleles, HLA-B alleles, and HLA-C alleles.

In any and all embodiments disclosed herein, generating the immunogenic cost metric may comprise determining, by the one or more processors, a geometric mean of probabilities of the p53-derived nonamer neopeptides including the p53 missense mutation binding each allele of the MHC class I molecules.

In any and all embodiments disclosed herein, the MHC class I molecules may comprise one or more HLA alleles selected from the group consisting of A*02:11, A*26:02, A*68:23, C*07:01, A*02:03, A*02:06, C*12:03, A*68:02, A*24:03, B*15:03, B*15:17, B*57:01, B*58:01, A*31:01, A*33:01, A*68:01, A*11:01, A*30:01, A*32:07, B*08:01, C*03:03, A*02:01, A*02:12, A*02:17, B*39:01, and B*73:01.

In any and all embodiments disclosed herein, the dataset may be generated, by the one or more processors, from DNA sequencing data obtained from one or more patients diagnosed with or at risk for Li-Fraumeni syndrome (LFS).

In any and all embodiments disclosed herein, the tumor behavior classification may identify the tumor type as corresponding to colorectal cancer, lung cancer, breast cancer, ovarian cancer, uterine cancer, or thyroid cancer.

In any and all embodiments disclosed herein, the plurality of p53 missense mutations may comprise germline p53 mutations.

In another aspect, the present disclosure provides a computing device comprising one or more processors and a computer-readable memory with instructions executable by the one or more processors to cause the computing device to perform steps for selecting a candidate therapy based on mutant p53 fitness, the steps comprising: (a) obtaining a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating an immunogenic cost metric for the p53 missense mutation based on binding affinities of MHC class I molecules to p53-derived nonamer neopeptides including the p53 missense mutation; and (iii) generating, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying a subset of p53 missense mutations that have fitness scores that exceed a threshold; (d) selecting adoptive T-cell therapy or neoantigen vaccine therapy for the subset of p53 missense mutations; and (e) storing, in a non-volatile memory device, adoptive T-cell therapy or neoantigen vaccine therapy in association with the subset of p53 missense mutations as a candidate therapy. Additionally or alternatively, in some embodiments, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

In another aspect, the present disclosure provides a computing device comprising one or more processors and a computer-readable memory with instructions executable by the one or more processors to cause the computing device to perform steps for selecting a candidate anti-cancer therapy based on mutant p53 fitness, the steps comprising: (a) obtaining a dataset comprising a plurality of p53 missense mutations present in one or more subjects; (b) for each p53 missense mutation in the dataset, applying a model to obtain a fitness score, wherein the model comprises: (i) generating an immunogenic cost metric for the p53 missense mutation based on binding affinities of MHC class I molecules to p53-derived nonamer neopeptides including the p53 missense mutation; and (ii) generating, based on the immunogenic cost metric, a fitness score, wherein generating the fitness score comprises applying a divergence-based statistical analysis to optimize the immunogenic cost metric; (c) identifying a subset of p53 missense mutations that have fitness scores that fall below a threshold; (d) selecting an immune checkpoint blockade therapy for the subset of p53 missense mutations; and (e) storing, in a non-volatile memory device, the immune checkpoint blockade therapy in association with the subset of p53 missense mutations as a candidate anti-cancer therapy. Additionally or alternatively, in some embodiments, the model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

In another aspect, the present disclosure provides a computing device comprising one or more processors and a computer-readable memory with instructions executable by the one or more processors to cause the computing device to perform steps for classifying tumor behavior for a potential tumor based on mutant p53 fitness, steps comprising: (a) obtaining a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating an immunogenic cost metric for the p53 missense mutation based on binding affinities of MHC class I molecules to p53-derived nonamer neopeptides including the p53 missense mutation; and (iii) generating, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying a subset of p53 missense mutations that have fitness scores that exceed a threshold; (d) identifying at least one of an age of tumor onset or a tumor type corresponding to the potential tumor for the subset of p53 missense mutations; and (e) storing, in a non-volatile memory device, the at least one of the age of tumor onset or the tumor type in association with the subset of p53 missense mutations as a tumor behavior classification. Additionally or alternatively, in some embodiments, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

### BRIEF DESCRIPTION OF THE DRAWINGS

FIGS. 1A-1H: Particular driver gene hotspots are highly conserved and avoid neoantigen presentation. FIG. 1A: Left panel: rank correlation between shared mutation frequencies in TCGA and the COSMIC database for commonly mutated tumor suppressors and oncogenes plotted versus the −log 10 of the rank correlation p-value—gene names are annotated. All points except MSH2 and MSH3 correspond to p-value <0.05. Right panel: correlation of individual hotspot mutation frequencies in TCGA and the COSMIC database (Pearson r=0.904, p-value=7.106e-24; Spearman r=0.908, p-value=2.332e-24). FIG. 1B: Comparison of TP53 mutation distributions in TCGA (N=2,764) and IARC (N=20,712) databases (Pearson r=0.97, p-value <0.0001; Spearman r=0.6, p-value <0.0001)—key hotspots are labelled. FIG. 1C: Comparison of conservation in hotspots and other mutations in the same gene (Welch's T-test p-value, p-value <0.05 are annotated). FIG. 1D: Comparison of neoantigen presentation between hotspots and other mutations in the same gene (Welch's T-test p-value, p-value <0.05 are labeled). FIG. 1E: p-values corresponding to panels FIG. 1C and FIG. 1D plotted against each other, genes in the upper right have hotspots which are both significantly conserved and avoid neoantigen presentation. FIG. 1F: Median of inferred association constant for p53 transcription factor affinity across eight p53 transcriptional targets (WAF1, MDM2, BAX, h1433s, AIP1, GADD45, NOXA, P53R2) versus frequency of p53 mutations in TCGA (Pearson r=−0.204, p-value <0.0001; Spearman r=−0.404, p-value <0.0001). FIG. 1G: Effective inferred association constants of 9-mers surrounding TP53 mutations versus frequency of TP53 mutations in TCGA (Pearson r=−0.079, p-value=0.088; Spearman r=−0.053, p-value=0.256). Mutant p53 hotspots are indicated in the oval, association constant is defined as inverse of the dissociation constant. FIG. 1H: Scatter plot of mutant p53 functional and immune association constants showing a weak dependence between the two phenotypes (Pearson r=0.073, p-value=0.117; Spearman r=0.144, p-value=0.002)—hotspots are indicated by arrows.

FIGS. 2A-2F: Mutant p53 fitness model quantifies trade-off between loss of function and immunogenicity. FIG. 2A: Model with only background intrinsic mutational frequencies (KL divergence=1.222, Pearson r=0.324, p-value <0.0001; Spearman r=0.2, p-value <0.0001)—hotspots are indicated by ovals. FIG. 2B: Relationship between mutant p53 concentration (log 2-transformed) and predicted effective p53 association constant to MDM2 promoter DNA across TCGA. Concentrations are normalized across tumor heterogeneity and allele count. Effective association constants are inferred across all possible p53 tetramer species given cell-specific wild-type and mutant allele distributions (N=219, Pearson p-value <0.0001, Spearman p-value <0.0001). FIG. 2C: Correlation of predicted p53 mutation frequencies to observed frequencies on a permutation (KL divergence=0.599; Pearson r=0.671, p-value <0.0001; Spearman r=0.39, p-value <0.0001). FIG. 2D: Correlation of predicted p53 mutation frequencies to observed frequencies on per-protein position basis (KL divergence=0.337; Pearson r=0.794, p-value <0.0001; Spearman r=0.782, p-value <0.0001). FIG. 2E: Sum of the log of background frequencies and positive functional fitness (Intrinsic Fitness) versus negative immune fitness (Extrinsic Fitness) (Pearson r=−0.31, p-value <0.0001; Spearman r=−0.33, p-value <0.0001). White line corresponds to the Pareto front (R175 and R248 are annotated), silver star indicates optimal free fitness constrained by the Pareto front, and the heatmap corresponds to the distance to the front. FIG. 2F: Comparison of free fitness distributions of nonhotspot and hotspot mutations (p-value <0.0001, Welch's T-test).

FIGS. 3A-3F: Validation of differential reactivity to mutant p53 neoepitopes in cancer patients and healthy donors. FIGS. 3A-3B: PBMCs from patients with R175H and/or R248Q p53 mutant tumors (FIG. 25) were cultured with the indicated p53 neopeptides (FIG. 24), CEF, or DMSO as positive and negative controls, respectively. FIG. 3A: Flow cytometry quantification of IFN-γ±TNFα expressing cells among CD8+CD3+ live T cells in the indicated samples. DMSO data are mean±SD of 2-3 technical replicates. FIG. 3B: Assessment of IFN-g responses (IFN-g+ cells among CD8+ T cells) in the same samples as FIG. 3A, in association with frequencies of total CD8+ T cells in those cultures. Black arrows indicate reacting samples; white arrow indicates low input CD8+ T cells. FIGS. 3C-3F: Reactivity of PBMCs from healthy donors to the indicated TP53 mutations by an optimized ex vivo priming assay (FIGS. 3C-3D), and MIRA assay using TCR-sequencing to quantify specific T-cell clonal expansion (FIG. 3E). IFN-γ (FIG. 3C) and Ki67 (FIG. 3D) expression in total CD8 T-cell fraction (top) or non-naive memory CD8 T-cell fraction (bottom). Frequencies are shown for two individual healthy donors as % of live single cells in culture after two weeks of in vitro stimulation with indicated p53 neopeptides in comparison with CEF and DMSO or an HIV peptide pool as positive and negative controls, respectively. FIG. 3E: Quantification of reactive TCRs in 107 healthy donors in 222 MIRA assay experiments (Adaptive Biotech.), with an average of two experiments per donor. Median values denoted by a red horizontal line, and zero values are circled in red with the number of points annotated in blue. FIG. 3F: TP53 hotspots along the Pareto front yielding fewer or increased TCRs are grouped in red squares—hotspots are annotated in black. Statistical significance is assessed by unpaired two-sided T-test (FIGS. 3C-3D) or Mann-Whitney U-test (FIG. 3E). *p≤0.05, **p≤0.01, ***p≤0.001, ****p≤0.0001.

FIGS. 4A-4F: Mutant p53 fitness relates to non-neoplastic p53 mutation distribution and Li-Fraumeni Syndrome age of tumor onset. FIGS. 4A-4B: Kaplan-Meier curves are split on median mutant p53 fitness for age of tumor onset in the (FIG. 4A) IARC R20 germline dataset (N=998) and the (FIG. 4B) NCI LFS dataset (N=82). FIG. 4C: Comparison of TP53 mutation frequencies in non-neoplastic tissues (3,451 mutation occurrences) with respect to TCGA (2,764 mutation occurrences): Pearson r=0.732, p-value <0.0001; Spearman r=0.544, p-value <0.0001. The top ten non-neoplastic mutations are colored in red and annotated. FIG. 4D: Positive relationship between hotspot frequency difference between non-cancerous and cancerous cells and magnitude of immune fitness. CpG-associated hotspots and non-CpG-associated hotspot Y220C are indicated (Overall: Pearson r=0.594, p-value=0.120; Spearman r=0.619, p-value=0.102; CpG-associated hotspots only: Pearson r=0.827, p-value=0.022; Spearman r=0.786, p-value=0.036). FIG. 4E: Kullback-Leibler divergence plotted as a function of relative immune weight for the largest tissue-specific mutation distributions across collected non-neoplastic somatic p53 mutations. The optimal immune weights are denoted as stars. The optimal relative immune weight for TCGA is denoted as a black dotted line. FIG. 4F: Most explanatory models across mutant TP53 datasets is indicated with a dot.

FIG. 5: Inferred relationships between relative transactivation and apparent dimer dissociation constant. Non-linear relationship between relative transactivation and inferred apparent dimer dissociation constant for mutant dimer p53. Blue dotted lines correspond wild-type p53, which has a relative transactivation of 1 (Methods section: Relative transactivation yeast assays). The hotspots' inferred values are annotated.

FIGS. 6A-6C: Relationship between mutant p53 concentration and predicted MDM2 binding affinities. FIG. 6A: Variation in normalized concentration across mutant p53 versus predicted MDM2 DNA affinity in common p53-mutated tissues in TCGA. Protein concentration is expressed as log 2 of inferred protein concentration in nanomolar (nM) units. FIG. 6B: Fraction positive immunohistochemistry (IHC) assay derived from IARC R20 dataset plotted against predicted per-allele mutant p53 concentration averaged across tissues. Correlations are for mutations with at least 10 IHC data entries (Pearson p-value 0.00848, Spearman p-value 0.00967). FIG. 6C: Fraction positive IHC assay plotted against predicted per-allele mutant p53 concentration averaged across tissues only for the TP53 hotspots (Pearson p-value 0.0207, Spearman p-value 0.00503).

FIGS. 7A-7D: Fitness model prediction analysis. FIG. 7A: Predicted ratio from full fitness model plotted against posterior ratio for each TP53 mutation. Mutations are colored by their observed frequency. Ratios >1 are predicted to be fixed in the cancer population. Diagonal line corresponds to the ratios being equal. FIG. 7B: Prediction accuracy plotted as proportion of observed mutation frequency for true positive (TP), false positive (FP), true negative (TN), and false negative (FN) model predictions. FIG. 7C: Fixing model weights and increasing the number of simulated HLA haplotypes improves model predictions according to the mutation sample size. FIG. 7D: Internal validation by shuffling background mutation frequencies, functional phenotypes, and immune phenotypes of TP53 mutations for 1,000 iterations and computing the KL divergence for each iteration. Histogram is of distribution of Kullback-Leibler divergences from all iterations. Permutation mean KL divergence is plotted as a vertical black dotted line and the true Kullback-Leibler divergence is plotted as a vertical dotted line.

FIGS. 8A-8E: Fitness model predicts mutation frequencies in commonly mutated cancer driver genes. FIG. 8A: Degree to which models of varying complexity account for mutation distributions from TCGA and COSMIC across 27 commonly mutated cancer driver genes. Models are ranked by Bayesian Information Criterion (BIC) in descending order. FIG. 8B: Variance in mutation frequencies for models of different complexities. FIG. 8C: Fitness model results for PTEN, using both conservation and immunogenicity over background mutation rates. Full model is justified by the BIC for TCGA per protein position (KL divergence=0.269; Pearson r=0.701, p-value=2.013e-24; Spearman r=0.701, p-value=2.386e-24). FIG. 8D: Fitness model results for KRAS, using a full model with conservation, function, and immunogenicity over background mutation rates using functional information available for seven KRAS cancer hotspot mutations. All components are justified by the BIC for TCGA per mutation per protein position (KL divergence=0.256; Pearson r=0.981, p-value=2.095e-24; Spearman r=0.616, p-value=0.000104). FIG. 8E: Trade-off between gain-of-function and avoidance of antigen presentation in TCGA pancreatic cancer for KRAS hotspots (Pearson −0.750, p-value=2.599e-23; Spearman r=−0.774, p-value=1.507e-25).

FIGS. 9A-9G: Inferred mutant immunogenicity is not related to pathogenicity. FIGS. 9A-9F: Comparison of inferred immunogenicity across not-pathogenic and pathogenic missense mutations in nine non-cancerous disease driver genes (HBA, HBB, HBD, HG1, HG2, F8, PAH, PHEX, and POGZ). Six out of nine genes had sufficient data for comparison between not-pathogenic and pathogenic mutations (HBA, HBB, F8, PAH, PHEX, and POGZ). FIG. 9G: Data corresponding to all the hemoglobin subunits (HBA, HBB, HBD, HG1, HG2) was combined and compared (HEMOGLOBIN). Mutations and their “Not-Pathogenic” and “Pathogenic” status were determined using the NCBI's dbSNP and ClinVar systems, respectively.

FIGS. 10A-10D: Fitness trade-offs inferred from ATAC- and RNA-seq. FIG. 10A: Lack of binding score plotted versus predicted functional fitness. Most ATAC-seq samples are breast cancer (BRCA), therefore TCGA BRCA samples were only plot matched to normalize on tissue-specific protein abundance (Pearson r=0.46, p-value=0.063, Spearman r=0.55, p-value 0.023, N=17). FIG. 10B: log 2 of median RNA expression (TPM) of eight TP53 target genes utilized in fitness model split on median ATAC-seq lack of DNA binding score (Mann-Whitney p-value 0.006). FIG. 10C: Immune fitness plotted versus ATACseq-based lack of DNA binding footprinting score for each TCGA sample (Pearson r=−0.45, p-value <0.0001; Spearman r=−0.49, p-value <0.0001). FIG. 10D: log 2 of median RNA expression (TPM) of target genes (WAF1, BAX, h1433s, AIP1, GADD45, and NOXA) with available ATAC-seq data plotted versus median probability of mutant p53 binding DNA conditioned on target DNA chromatin accessibility (Pearson r=0.25, p-value 0.0459; Spearman r=0.088, p-value 0.480).

FIGS. 11A-11F: Differential T-cell reactivity to p53 neopeptides. FIG. 11A: Flow cytometry quantification of HLA-A*02:01 expression on the surface of live T2 cells as a measure of peptide:MHC stabilization via binding to specific peptides. T2 cells were incubated overnight in serum-free media with recombinant human B2M and the indicated peptides at the indicated concentrations, or DMSO as vehicle control. Negative controls (DMSO and unrelated HLA-B*35-restricted NY-ESO-1-derived peptide); positive controls (HLA-A*02:01-restricted peptides from flu and HIV viral antigens and Mart1/Melan-A melanoma-associated antigen); experimental peptides containing the indicated mutation in comparison with the corresponding wild-type (wt) sequence. Data are mean±SD of 2-3 replicates. p-values are calculated with a two-sided unpaired T-test. FIG. 11B: Model illustrating the molecular basis of the T-cell stimulation assay and stimulation conditions (APC, antigen presenting cell; TCR, T-cell receptor). FIG. 11C: Representative plots of IFN-γ±TNF-α expressing cells among CD8+CD3+ live T cells in PBMCs from patients with mutant p53 tumors as in FIGS. 3A, 3D, Correlation analyses between indicated parameters in PBMC samples from R248Q mutant patients with presence of disease at the time of PBMC collection as in FIGS. 3B, 3E, Estimate of mutant p53 amount per tumor cell before treatment in the same patients. Samples with R175H mutations are colored in blue. The sample which reacted is in solid blue, and the sample which did not react has filled-in lines. FIG. 3F: Flow cytometry gating strategy for total CD8 and non-naïve memory CD8 T cells analyzed in FIGS. 3C, 3D. TN: naïve T cells, TCM: central memory T cells, TEM, effector memory T cells, TEMRA: effector memory T cells reexpressing CD45RA. FIG. 11B discloses SEQ ID NOS: 1-3, respectively, in order of appearance.

FIGS. 12A-12C: Relationships between immune fitness and immune checkpoint protein expression in TCGA. FIGS. 12A-12B: Continuous and categorical relationships between both CTLA4 (FIG. 12A) and PD-1 (FIG. 12B) protein expression available from TCGA RPPA proteomics assay and immune fitness. Least-squares best-fit line plotted. For the CTLA4 scatterplot, Pearson p-value <0.0001, Spearman p-value <0.0001. For the PD-1 scatterplot, Pearson p-value=0.00153, Spearman p-value <0.0001. FIG. 12C: Continuous and categorical relationships between PD-L1 protein expression available from TCGA RPPA proteomics assay and immune fitness in commonly TP53-mutated tissues. Least-squares best-fit line plotted in red. Correlation p-values: Ovarian—Pearson p-value=0.2, Spearman p-value=0.0829; Colorectal—Pearson p-value=0.157, Spearman p-value 0.003; NSCLC—Pearson p-value=0.0812, Spearman p-value=0.00793; Breast—Pearson p-value=0.00671, Spearman p-value=0.000140.

FIGS. 13A-13B: p53 fitness predicts survival and immune relevance in diverse p53-mutated groups. FIG. 13A: Kaplan-Meier curves separated by median functional, immune, and total fitness in TCGA and non-small cell lung cancer (NSCLC) ICB-treated samples. Matched HLA-p53 mutations with lung-specific and allele-specific concentrations were used to determine functional, immune, and total fitness. NS p>0.05, *p≤0.05, **p≤0.01, ***p≤0.001, ****p≤0.0001. FIG. 13B: Log-rank score of TCGA (N=1941), NSCLC (N=289) and LFS cohorts (IARC N=898, NCI N=82) across the range of relative immune weights. Dashed horizonal line corresponds to a log-rank score for p-value 0.05 and the dashed vertical line marks the choice of parameters trained independently to best represent the observed mutation frequency in TCGA, which are the weights used for scoring of mutations in FIG. 13A, for the right-most column.

FIG. 14: Relationships of mutant fitness to non-neoplastic p53 mutations and germline p53 mutations. Kaplan-Meier curves separated by median functional and immune mutant p53 fitness for first-cancer age of onset in LFS IARC R20 germline dataset (N=998) and the NCI LFS cohort (N=82). Mutant p53 fitness was determined using TCGA-derived tissue-specific mutant p53 concentrations for both datasets, and individual HLA types for the NCI cohort and averages taken over simulated HLA types for the IARC dataset, which lacked individual HLA types.

FIG. 15: Correlation of observed mutation frequencies to expected intrinsic background mutation frequencies. Comparison of the expected background dinucleotide mutation frequencies and the observed mutation frequencies of selected cancer driver genes in TCGA.

FIG. 16: Additional fitness model results on specific hotspots. Distributions of predicted HLA-I haplotype-specific frequency values (Methods, Eq. 6) for each of the hotspot mutations for the TCGA pan-cancer model. The distributions are computed across haplotypes of patients in TCGA, where different HLA-I haplotypes correspond to different levels of immune selection. The HLA-I haplotype averaged frequencies are marked with dashed red lines, the observed frequencies are marked with vertical dashed green lines, and the horizontal dashed green lines correspond to 95% confidence intervals of the observed mutation frequency.

FIGS. 17A-17J: Heterogeneity and inferred mutant p53 concentration. FIG. 17A: Distribution of wild-type p53 concentration used for transforming RPPA values to concentration values. FIG. 17B: Distribution of mutant p53 concentration across mutations and tissues. FIGS. 17C-17D: Distribution of mutant (MT) and total number of TP53 alleles across TCGA. FIG. 17E: Cancer cell fraction distribution of TP53 mutations. FIGS. 17F-17G: Relationships between TP53 and MDM2 RNA and inferred p53 protein expression.

FIGS. 17H-17I: Distribution of mutant and fraction of mutant alleles across different TCGA tissues. FIG. 17J: Distribution of inferred mutant p53 concentration across TCGA tissues.

FIG. 18: Relationships between haplotype populations. Highly-correlated shared HLA-I frequencies in simulated and TCGA MHC-I haplotype populations.

FIGS. 19A-19C: Relationships between inferred mutant p53 conservation, stability, and mutation frequency in additional models. FIGS. 19A-19B: Relationship between conservation, stability and mutation frequency. Most hotspots are conserved and induce protein instability. The temperature used for the stability calculations is 310 K, approximately human body temperature. FIG. 19C: Relationship between conservation and protein stability.

FIG. 20A is a block diagram depicting an embodiment of a network environment comprising a client device in communication with server device.

FIG. 20B is a block diagram depicting a cloud computing environment comprising client device in communication with cloud service providers.

FIGS. 20C and 20D are block diagrams depicting embodiments of computing devices useful in connection with the methods and systems described herein.

FIG. 21 depicts a system that includes a computing device and a sample processing system according to various potential embodiments.

FIG. 22 depicts well-established pan-cancer TP53 hotspots whose order is conserved across databases

FIG. 23 shows comparison of the performance of the models in predicting the observed mutation frequencies in tumors

FIG. 24 shows summary of the binding affinities of test peptides when loaded onto autologous antigen presenting cells. Figure discloses SEQ ID NOS 4-8, 1, 9 and 2-3, respectively, in order of appearance.

FIG. 25 depicts the ability of R175H and R248Q/W TP53 hotspot mutations to elicit immune responses in cancer patients in vivo.

FIG. 26 shows the ability of the models to predict TP53 mutation distribution in a neoplastic setting.

FIG. 27 shows HLA molecules predicted to present hotspot TP53 peptides using NetMHC 3.4. Figure discloses SEQ ID NOS 50, 3, 51-52 and 2, respectively, in order of appearance.

FIG. 28 shows a summay of distinct 9-11 length peptide epitopes that encompassed common p53 mutations at positions pR175 (H), pR248 (Q), pR273 (C/H/L), and pR282 (W) that were predicted to bind to at least one of 60 common HLA class I alleles by NetMHCpan version 4.1. Figure discloses SEQ ID NOS 10-15, 10, 15, 11-14, 16-24, 19, 21, 20, 23-24, 22, 16-18, 25-45, 27, 44, 30-31, 33, 32, 25-26, 34-35, 38, 37, 29, 36, 28, 39, 41, 40, 43, 42, 45-49, 47-48, 46 and 49, respectively, in order of appearance.

### DETAILED DESCRIPTION OF THE DRAWINGS

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The methods disclosed herein are useful in determining whether a patient harboring TP53 mutations will benefit from immune checkpoint inhibitor therapy. Further, the methods of the present technology are useful in predicting the clinical phenotypes and/or age of tumor onset in a patient diagnosed with or at risk for LFS and comprising germline p53 mutations.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the terms “amplify” or “amplification” with respect to nucleic acid sequences, refer to methods that increase the representation of a population of nucleic acid sequences in a sample. Nucleic acid amplification methods, such as PCR, isothermal methods, rolling circle methods, etc., are well known to the skilled artisan. Copies of a particular nucleic acid sequence generated in vitro in an amplification reaction are called “amplicons” or “amplification products”.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is colorectal cancer, lung cancer, breast cancer, ovarian cancer, uterine cancer, or thyroid cancer.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a normal adjacent tumor (NAT), or any other non-cancerous sample from the same or a different subject.

“Detecting” as used herein refers to determining the presence of a mutation or alteration in a nucleic acid of interest in a sample. Detection does not require the method to provide 100% sensitivity.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “epitopes” refer to a class of major histocompatibility complex (MHC) bounded peptides that are recognized by the immune system as targets for T cells and can elicit an immune response in a subject. “Neoepitopes” refer to epitopes that arise from tumor-specific mutations that may elicit an immune response to cancer. Epitopes usually consist of chemically active surface groupings of molecules such as amino acids or sugar side chains and usually have specific three dimensional structural characteristics, as well as specific charge characteristics.

As used herein, “fitness” of a p53 mutation refers to the probability or propensity of a p53 mutation to be naturally selected and propagated during tumor evolution.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are described in Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” are used interchangeably and refer to an individual organism, a vertebrate, a mammal, or a human. In a preferred embodiment, the individual, patient or subject is a human.

As used herein, “major histocompatibility complex (MHC)” refers to a group of genes that code for proteins found on the surfaces of cells that help the immune system recognize foreign substances. MHC proteins are found in all higher vertebrates. In human beings the complex is also called the human leukocyte antigen (HLA) system. HLAs corresponding to MHC class I (A, B, and C) which all are the HLA Class 1 group present peptides from inside the cell. In general, these particular peptides are small polymers, about 9 amino acids in length. Foreign antigens presented by MHC class I attract killer T-cells (also called CD8 positive- or cytotoxic T-cells) that destroy cells. HLAs corresponding to MHC class II (DP, DM, DO, DQ, and DR) present antigens from outside of the cell to T-lymphocytes. These particular antigens stimulate the multiplication of T-helper cells (also called CD4 positive T cells), which in turn stimulate antibody-producing B-cells to produce antibodies to that specific antigen. Self-antigens are suppressed by regulatory T cells.

As used herein, a “mutation” of a gene refers to the presence of a variation within the gene or gene product that affects the expression and/or activity of the gene or gene product as compared to the normal or wild-type gene or gene product. The genetic mutation can result in changes in the quantity, structure, and/or activity of the gene or gene product in a cancer tissue or cancer cell, as compared to its quantity, structure, and/or activity, in a normal or healthy tissue or cell (e.g., a control). For example, a mutation can have an altered nucleotide sequence (e.g., a mutation), amino acid sequence, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene. In certain embodiments, the mutations are associated with a phenotype, e.g., a cancerous phenotype (e.g., one or more of cancer risk, oncogenesis, immunogenicity, or responsiveness to treatment). In one embodiment, the mutation is associated with one or more of: a genetic risk factor for cancer, a positive treatment response predictor, a negative treatment response predictor, a positive prognostic factor, a negative prognostic factor, or a diagnostic factor. As used herein, a “missense mutation” refers to a mutation in which a single nucleotide substitution alters the genetic code in a way that produces an amino acid that is different from the usual amino acid at that position. In some embodiments, missense mutations alter one or more functions or physical-chemical properties of the encoded protein.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to a nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a methylated target nucleic acid. Probes are typically at least about 10, 15, 20, 25, 30, 35, 40, 50, 60, 75, 100 nucleotides or more in length.

The term “promoter region” of a target gene as used herein refers to a segment of the target gene to which RNA polymerase can bind to and initiate transcription of the target gene. In some embodiments, the promoter region may include the first 250 nucleotides (nt), first 300 nt, first 350 nt, first 400 nt, first 450 nt, first 500 nt, first 1 kb, first 5 kb, first 10 kb, first 15 kb, first 20 kb, first 21 kb or first 22 kb of genomic sequence directly upstream of the translation start site of target gene.

As used herein, a “sample” refers to a substance that is being assayed for the presence of a mutation in a nucleic acid of interest. Processing methods to release or otherwise make available a nucleic acid for detection may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 420° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target gene”, “target sequence” and “target nucleic acid sequence” refer to a specific nucleic acid sequence to be detected and/or quantified in the sample to be analyzed.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission. In some embodiments, “inhibiting,” means reducing or slowing the growth of a tumor. In some embodiments, the inhibition of tumor growth may be, for example, by 5% or more, 10% or more, 20% or more, 30% or more, 40% or more, 50% or more, 60% or more, 70% or more, 80% or more, or 90% or more. In some embodiments, the inhibition may be complete.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Systems, Devices, and Methods for Modeling Fitness of p53 Mutations

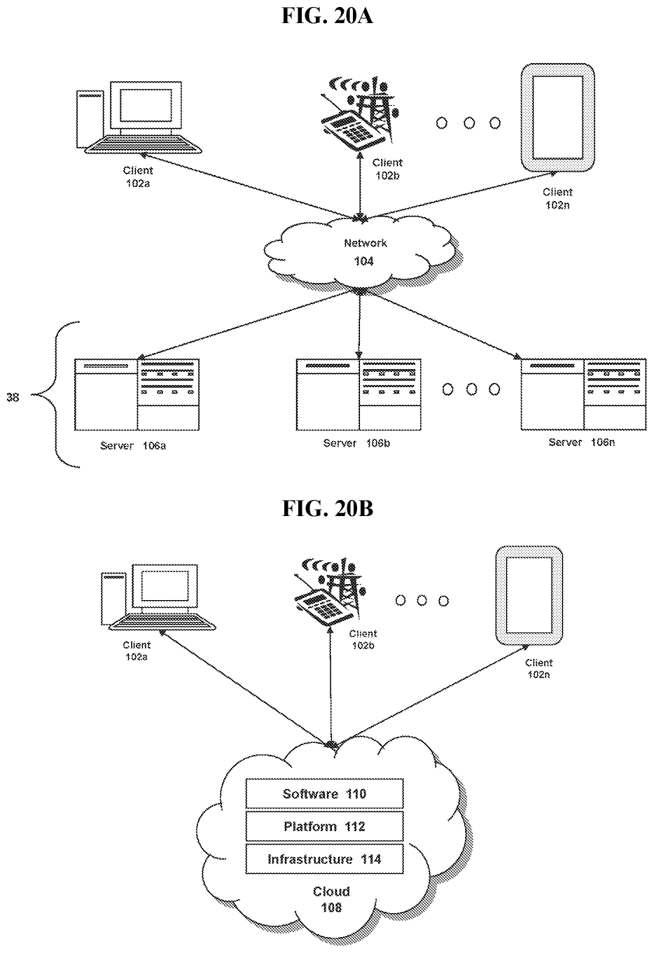

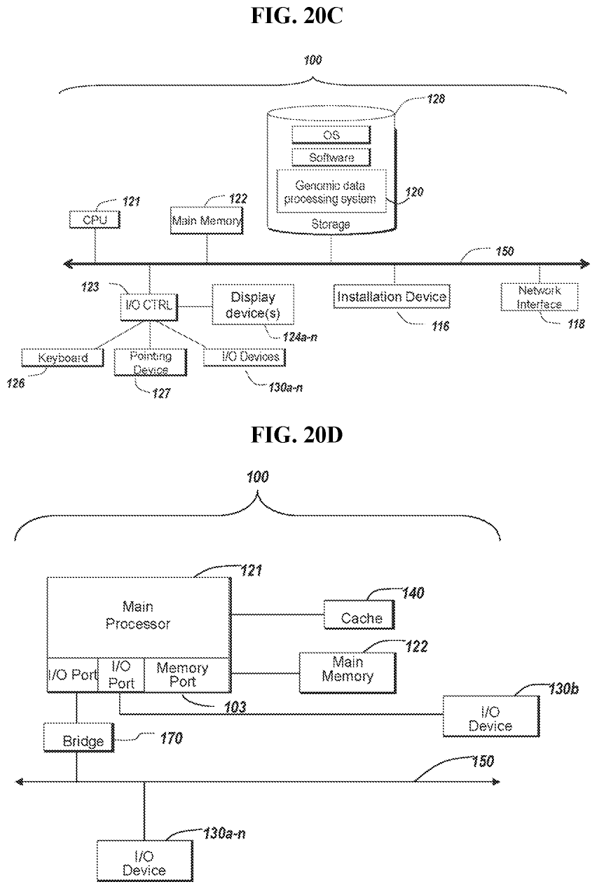

Aspects of the operating environment as well as associated system components (e.g., hardware elements) in connection with various embodiments of the methods and systems described herein will now be discussed. Referring to FIG. 20A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 20A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 20B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 20C and 20D depict block diagrams of a computing device 100 useful for practicing an embodiment of the client 102 or a server 106. As shown in FIGS. 20C and 20D, each computing device 100 includes a central processing unit 121, and a main memory unit 122. As shown in FIG. 20C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 20D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit or memory device 122 may include one or more memory chips capable of storing data and allowing any storage location to be directly accessed by the microprocessor 121. Main memory unit or device 122 may be volatile and faster than storage 128 memory. Main memory units or devices 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 20C, the processor 121 communicates with main memory 122 via a system bus 150 (described in more detail below). FIG. 20D depicts an embodiment of a computing device 100 in which the processor communicates directly with main memory 122 via a memory port 103. For example, in FIG. 20D the main memory 122 may be DRDRAM.

FIG. 20D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 20D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 20D depicts an embodiment of a computer 100 in which the main processor 121 communicates directly with I/O device 130b or other processors 121′ via HYPERTRANSPORT, RAPIDIO, or INFINIBAND communications technology. FIG. 20D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 20C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 20C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 20B and 20C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

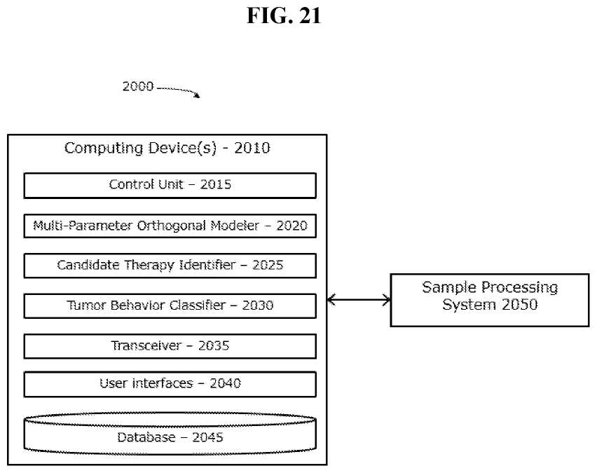

Referring to FIG. 21, in various embodiments, a system 2000 may include a computing device 2010 (or multiple computing devices, co-located or remote to each other) and a sample processing system 2050. In various embodiments, computing device 2010 (or components thereof) may be integrated with the sample processing system 2050 (or components thereof). In various embodiments, the sample processing system 2050 may include, may be, or may employ, in situ hybridization, PCR, Next-generation sequencing, Northern blotting, microarray, dot or slot blots, FISH, electrophoresis, chromatography, and/or mass spectroscopy on such biological sample as blood, plasma, serum, and/or tissue. For example, in certain embodiments, the sample processing system 2050 may be or may include a Next-generation sequencer.

The computing device 110 (or multiple computing devices) may be used to control, and receive signals acquired via, components of sample processing system 2050. The computing device 110 may include one or more processors and one or more volatile and non-volatile memories for storing computing code and data that are captured, acquired, recorded, and/or generated. The computing device 110 may include a control unit 114 that is configured to exchange control signals with sample processing system 2050, allowing the computing device 110 to be used to control, for example, processing of samples and/or delivery of data generated and/or acquired through processing of samples. An orthoganol modeler 2020 may be used, for example, to perform analyses of data captured using sample processing system 150, and may include, for example, generating various metrics and fitness scores as discussed herein. A candidate therapy identifier 2025 and/or a tumor behavior classifier 2030 may use analysis performed via modeler 2020 to, for example, select candidate therapies and/or classify tumor behavior for potential tumors (e.g., make predictions regarding age of onset and/or tumor type).

A transceiver 2035 allows the computing device 2010 to exchange readings, control commands, and/or other data with sample processing system 2050 (or components thereof). One or more user interfaces 2040 allow the computing device 2010 to receive user inputs (e.g., via a keyboard, touchscreen, microphone, camera, etc.) and provide outputs (e.g., via display screen, audio speakers, etc.). The computing device 2010 may additionally include one or more databases 2045 (stored in, e.g., on or more computer-readable non-volatile memory devices) for storing, for example, data and analyses obtained via multi-parameter orthogonal modeler 2020, candidate therapy identifier 2025, tumor behavior classifier 2030, and/or sample processing system 2050. In some implementations, database 2045 (or portions thereof) may alternatively or additionally be part of another computing device that is co-located or remote and in communication with computing device 2010 and/or sample processing system 2050 (or components thereof).

In one aspect, the present disclosure provides a method for selecting a candidate therapy based on mutant p53 fitness, comprising: (a) obtaining or otherwise receiving, by one or more processors of a computing device, a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating, by one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating, by one or more processors, an immunogenic cost metric for the p53 missense mutation based on binding affinities of MHC class I molecules to p53-derived nonamer neopeptides including the p53 missense mutation; and (iii) generating, by one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying, by one or more processors, a subset of p53 missense mutations that have fitness scores that exceed a threshold; (d) selecting adoptive T-cell therapy or neoantigen vaccine therapy for the subset of p53 missense mutations; and (e) storing, by one or more processors, in a computer-readable non-volatile memory device, adoptive T-cell therapy or neoantigen vaccine therapy in association with the subset of p53 missense mutations as a candidate therapy. The neoantigen vaccine therapy may be a RNA neoantigen vaccine, a synthetic long peptide neoantigen vaccine, or a dendritic cell (DC)-based neoantigen vaccine. In some embodiments, the pro-oncogenic advantage metric is assigned a greater weight relative to the immunogenic cost metric. Additionally or alternatively, in some embodiments, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

Additionally or alternatively, in some embodiments, the method further comprises administering the adoptive T-cell therapy or neoantigen vaccine therapy to a patient comprising at least one p53 missense mutation that is present in the subset of p53 missense mutations. In any of the above embodiments of the methods disclosed herein, the dataset is generated from DNA sequencing data obtained from one or more patients diagnosed with or at risk for cancer or Li-Fraumeni syndrome (LFS). Examples of cancer include, but are not limited to, colorectal cancer, lung cancer, breast cancer, ovarian cancer, uterine cancer, or thyroid cancer.

Additionally or alternatively, in certain embodiments, the at least one p53 target gene is WAF1, MDM2, BAX, h1433s, AIP1, GADD45, NOXA, or P53R2. In some embodiments, the transactivation levels of the at least one p53 target gene are determined using quantitative transactivation assays in yeast.

In any of the preceding embodiments disclosed herein, the pro-oncogenic advantage metric is a median probability of the p53 polypeptide encoded by the p53 missense mutation not binding to the promoter region of the at least one p53 target gene. In some embodiments, generating the pro-oncogenic advantage metric comprises applying a cooperative Hill function.

In another aspect, the present disclosure provides a method for selecting a candidate anti-cancer therapy based on mutant p53 fitness, comprising: (a) obtaining or otherwise receiving, by one or more processors of a computing device, a dataset comprising a plurality of p53 missense mutations present in one or more subjects; (b) for each p53 missense mutation in the dataset, applying a model to obtain a fitness score, wherein the model comprises: (i) generating, by one or more processors, an immunogenic cost metric for the p53 missense mutation based on binding affinities of MHC class I molecules to p53-derived nonamer neopeptides including the p53 missense mutation; and (ii) generating, by one or more processors, based on the immunogenic cost metric, a fitness score, wherein generating the fitness score comprises applying a divergence-based statistical analysis to optimize the immunogenic cost metric; (c) identifying, by one or more processors, a subset of p53 missense mutations that have fitness scores that fall below a threshold; (d) selecting, by one or more processors, an immune checkpoint blockade therapy for the subset of p53 missense mutations; and (e) storing, by one or more processors, in a computer-readable non-volatile memory, the immune checkpoint blockade therapy in association with the subset of p53 missense mutations as a candidate anti-cancer therapy. Additionally or alternatively, in some embodiments, the model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

Additionally or alternatively, in some embodiments, the method further comprises administering the immune checkpoint blockade therapy to a cancer patient comprising at least one p53 missense mutation that is present in the subset of p53 missense mutations. Examples of immune checkpoint blockade therapy include, but are not limited to, anti-PD-1 antibodies, anti-PD-L1 antibodies, anti-PD-L2 antibodies, anti-CTLA-4 antibodies, anti-TIM3 antibodies, anti-TIGIT antibodies, anti-VISTA antibodies, anti-B7-H3 antibodies, anti-BTLA antibodies, anti-CD73 antibodies, or anti-LAG-3 antibodies. In any of the above embodiments disclosed herein, the dataset is generated from DNA sequencing data obtained from one or more patients diagnosed with or at risk for cancer. Examples of cancer include, but are not limited to, colorectal cancer, lung cancer, breast cancer, ovarian cancer, uterine cancer, or thyroid cancer.

In any and all embodiments disclosed herein, the divergence-based statistical analysis comprises minimizing divergence scores between observed and predicted frequencies of the p53 missense mutation. In certain embodiments, the divergence scores that are minimized are Kullback-Leibler divergences.

Additionally or alternatively, in some embodiments disclosed herein, the MHC class I molecules comprise HLA-A alleles, HLA-B alleles, and HLA-C alleles. In some embodiments, generating the immunogenic cost metric comprises determining a geometric mean of probabilities of the p53-derived nonamer neopeptides including the p53 missense mutation binding each allele of the MHC class I molecules. Additionally or alternatively, in some embodiments disclosed herein, the MHC class I molecules comprise one or more HLA alleles selected from the group consisting of A*02:11, A*26:02, A*68:23, C*07:01, A*02:03, A*02:06, C*12:03, A*68:02, A*24:03, B*15:03, B*15:17, B*57:01, B*58:01, A*31:01, A*33:01, A*68:01, A*11:01, A*30:01, A*32:07, B*08:01, C*03:03, A*02:01, A*02:12, A*02:17, B*39:01, and B*73:01.

In any and all embodiments disclosed herein, the plurality of p53 missense mutations comprises somatic and/or germline p53 mutations.

In another aspect, the present disclosure provides a method for classifying tumor behavior for a potential tumor based on mutant p53 fitness, comprising: (a) obtaining or otherwise receiving, by one or more processors of a computing device, a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating, by the one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating, by the one or more processors, an immunogenic cost metric for the p53 missense mutation based on binding affinities of MHC class I molecules to p53-derived nonamer neopeptides including the p53 missense mutation; and (iii) generating, by the one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying, by the one or more processors, a subset of p53 missense mutations that have fitness scores that exceed a threshold; (d) identifying, by the one or more processors, at least one of an age of tumor onset or a tumor type corresponding to the potential tumor for the subset of p53 missense mutations; and (e) storing, by the one or more processors, in a computer-readable non-volatile memory device, the at least one of the age of tumor onset or the tumor type in association with the subset of p53 missense mutations as a tumor behavior classification. Additionally or alternatively, in some embodiments, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

In any and all of the embodiments disclosed herein, the tumor behavior classification identifies the age of tumor onset as, for example, 10-20 years, or as 30-50 years, or as 50 years or older. In any and all of the embodiments disclosed herein, the pro-oncogenic advantage metric is assigned a greater weight relative to the immunogenic cost metric. In any and all of the embodiments disclosed herein, the at least one p53 target gene is WAF1, MDM2, BAX, h1433s, AIP1, GADD45, NOXA, or P53R2. In any and all of the embodiments disclosed herein the transactivation levels of the at least one p53 target gene are determined using quantitative transactivation assays in yeast. In any and all of the embodiments disclosed herein, the pro-oncogenic advantage metric is a median probability of the p53 polypeptide encoded by the p53 missense mutation not binding to the promoter region of the at least one p53 target gene. In any and all of the embodiments disclosed herein generating the pro-oncogenic advantage metric comprises applying, by the one or more processors, a cooperative Hill function. In any and all of the embodiments disclosed herein, the divergence-based statistical analysis comprises minimizing, by the one or more processors, divergence scores between observed and predicted frequencies of the p53 missense mutation. In any and all of the embodiments disclosed herein, the divergence scores that are minimized are Kullback-Leibler divergences. In any and all of the embodiments disclosed herein, the MHC class I molecules comprise HLA-A alleles, HLA-B alleles, and HLA-C alleles. In any and all of the embodiments disclosed herein, generating the immunogenic cost metric comprises determining, by the one or more processors, a geometric mean of probabilities of the p53-derived nonamer neopeptides including the p53 missense mutation binding each allele of the MHC class I molecules. In any and all of the embodiments disclosed herein, the MHC class I molecules comprise one or more HLA alleles selected from the group consisting of A*02:11, A*26:02, A*68:23, C*07:01, A*02:03, A*02:06, C*12:03, A*68:02, A*24:03, B*15:03, B*15:17, B*57:01, B*58:01, A*31:01, A*33:01, A*68:01, A*11:01, A*30:01, A*32:07, B*08:01, C*03:03, A*02:01, A*02:12, A*02:17, B*39:01, and B*73:01. In any and all of the embodiments disclosed herein, the dataset is generated, by the one or more processors, from DNA sequencing data obtained from one or more patients diagnosed with or at risk for Li-Fraumeni syndrome (LFS). In any and all of the embodiments disclosed herein, the tumor behavior classification identifies the tumor type as corresponding to colorectal cancer, lung cancer, breast cancer, ovarian cancer, uterine cancer, or thyroid cancer. In any and all of the embodiments disclosed herein, the plurality of p53 missense mutations comprises germline p53 mutations.

In another aspect, the present disclosure provides a method for predicting fitness of p53 mutations, comprising: (a) obtaining, by one or more processors of a computing device, a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating, by the one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating, by the one or more processors, an immunogenic cost metric for the p53 missense mutation based on binding affinities of MHC class I molecules to p53-derived nonamer neopeptides including the p53 missense mutation; and (iii) generating, by the one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying, by the one or more processors, a subset of p53 missense mutations that have fitness scores that exceed a threshold as high-fitness p53 mutations and/or identifying, by the one or more processors, a subset of p53 missense mutations that have fitness scores that fall below a threshold as low-fitness p53 mutations.

In another aspect, the present disclosure provides one or more computing devices comprising one or more processors and a computer-readable storage medium with instructions executable by the one or more processors to cause the computing device to perform steps for predicting fitness of p53 mutations, said steps comprising: (a) obtaining, by one or more processors of a computing device, a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating, by the one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating, by the one or more processors, an immunogenic cost metric for the p53 missense mutation based on binding affinities of MHC class I molecules to p53-derived nonamer neopeptides including the p53 missense mutation; and (iii) generating, by the one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying, by the one or more processors, a subset of p53 missense mutations that have fitness scores that exceed a threshold as high-fitness p53 mutations and/or identifying, by the one or more processors, a subset of p53 missense mutations that have fitness scores that fall below a threshold as low-fitness p53 mutations.

In another aspect, the present disclosure provides one or more computing devices comprising one or more processors and a computer-readable storage medium with instructions executable by the one or more processors to cause the computing device to perform steps for selecting a candidate therapy based on mutant p53 fitness, the steps comprising: (a) obtaining a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating an immunogenic cost metric for the p53 missense mutation based on binding affinities of MHC class I molecules to p53-derived nonamer neopeptides including the p53 missense mutation; and (iii) generating, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying a subset of p53 missense mutations that have fitness scores that exceed a threshold; (d) selecting adoptive T-cell therapy or neoantigen vaccine therapy for the subset of p53 missense mutations; and (e) storing, in a non-volatile memory device, adoptive T-cell therapy or neoantigen vaccine therapy in association with the subset of p53 missense mutations as a candidate therapy. Additionally or alternatively, in some embodiments, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

In another aspect, the present disclosure provides one or more computing devices comprising one or more processors and a computer-readable storage medium with instructions executable by the one or more processors to cause the computing device to perform steps for selecting a candidate anti-cancer therapy based on mutant p53 fitness, the steps comprising: (a) obtaining a dataset comprising a plurality of p53 missense mutations present in one or more subjects; (b) for each p53 missense mutation in the dataset, applying a model to obtain a fitness score, wherein the model comprises: (i) generating an immunogenic cost metric for the p53 missense mutation based on binding affinities of MHC class I molecules to p53-derived nonamer neopeptides including the p53 missense mutation; and (ii) generating, based on the immunogenic cost metric, a fitness score, wherein generating the fitness score comprises applying a divergence-based statistical analysis to optimize the immunogenic cost metric; (c) identifying a subset of p53 missense mutations that have fitness scores that fall below a threshold; (d) selecting an immune checkpoint blockade therapy for the subset of p53 missense mutations; and (e) storing, in a non-volatile memory device, the immune checkpoint blockade therapy in association with the subset of p53 missense mutations as a candidate anti-cancer therapy. Additionally or alternatively, in some embodiments, the model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

In another aspect, the present disclosure provides one or more computing devices comprising one or more processors and a computer-readable storage medium with instructions executable by the one or more processors to cause the computing device to perform steps for classifying tumor behavior for a potential tumor based on mutant p53 fitness, steps comprising: (a) obtaining a dataset comprising a plurality of p53 missense mutations present in one or more subjects; (b) for each p53 missense mutation in the dataset, applying a multi-parameter orthogonal model to obtain a fitness score, wherein the multi-parameter orthogonal model comprises: (i) generating a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene; (ii) generating an immunogenic cost metric for the p53 missense mutation based on binding affinities of MHC class I molecules to p53-derived nonamer neopeptides including the p53 missense mutation; and (iii) generating, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric; (c) identifying a subset of p53 missense mutations that have fitness scores that exceed a threshold; (d) identifying at least one of an age of tumor onset or a tumor type corresponding to the potential tumor for the subset of p53 missense mutations; and (e) storing, in a non-volatile memory device, the at least one of the age of tumor onset or the tumor type in association with the subset of p53 missense mutations as a tumor behavior classification. Additionally or alternatively, in some embodiments, the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric.

In another aspect, the present disclosure provides a method for predicting fitness of PTEN mutations, comprising: (a) obtaining, by one or more processors of a computing device, a dataset comprising a plurality of PTEN missense mutations present in one or more subjects; (b) for each PTEN missense mutation in the dataset, applying a model to obtain a fitness score, wherein the model comprises: (i) generating, by the one or more processors, a conservation metric for the PTEN missense mutation based on an evolutionary rate assigned to each amino acid of a polypeptide encoded by the PTEN missense mutation; (ii) generating, by the one or more processors, an immunogenic cost metric for the PTEN missense mutation based on binding affinities of MHC class I molecules to PTEN-derived nonamer neopeptides including the PTEN missense mutation; and (iii) generating, by the one or more processors, based on the conservation metric and the immunogenic cost metric, a fitness score, wherein generating the fitness score comprises assigning weights to the conservation metric and the immunogenic cost metric, and applying a divergence-based statistical analysis to optimize the conservation metric and the immunogenic cost metric; and (c) identifying, by the one or more processors, a subset of PTEN missense mutations that have fitness scores that exceed a threshold as high-fitness PTEN mutations and/or identifying, by the one or more processors, a subset of PTEN missense mutations that have fitness scores that fall below a threshold as low-fitness PTEN mutations.

In another aspect, the present disclosure provides one or more computing devices comprising one or more processors and a computer-readable storage medium with instructions executable by the one or more processors to cause the computing device to perform steps for predicting fitness of PTEN mutations, said steps comprising: (a) obtaining, by one or more processors of a computing device, a dataset comprising a plurality of PTEN missense mutations present in one or more subjects; (b) for each PTEN missense mutation in the dataset, applying a model to obtain a fitness score, wherein the model comprises: (i) generating, by the one or more processors, a conservation metric for the PTEN missense mutation based on an evolutionary rate assigned to each amino acid of a polypeptide encoded by the PTEN missense mutation; (ii) generating, by the one or more processors, an immunogenic cost metric for the PTEN missense mutation based on binding affinities of MHC class I molecules to PTEN-derived nonamer neopeptides including the PTEN missense mutation; and (iii) generating, by the one or more processors, based on the conservation metric and the immunogenic cost metric, a fitness score, wherein generating the fitness score comprises assigning weights to the conservation metric and the immunogenic cost metric, and applying a divergence-based statistical analysis to optimize the conservation metric and the immunogenic cost metric; and (c) identifying, by the one or more processors, a subset of PTEN missense mutations that have fitness scores that exceed a threshold as high-fitness PTEN mutations and/or identifying, by the one or more processors, a subset of PTEN missense mutations that have fitness scores that fall below a threshold as low-fitness PTEN mutations.

In another aspect, the present disclosure provides a method for predicting fitness of KRAS mutations, comprising: (a) obtaining, by one or more processors of a computing device, a dataset comprising a plurality of KRAS missense mutations present in one or more subjects; (b) for each KRAS missense mutation in the dataset, applying a model to obtain a fitness score, wherein the model comprises: (i) generating, by the one or more processors, a conservation metric for the KRAS missense mutation based on an evolutionary rate assigned to each amino acid of a polypeptide encoded by the KRAS missense mutation; (ii) generating, by the one or more processors, an immunogenic cost metric for the KRAS missense mutation based on binding affinities of MHC class I molecules to KRAS-derived nonamer neopeptides including the KRAS missense mutation; (iii) generating, by the one or more processors, a pro-oncogenic advantage metric for the KRAS missense mutation based on binding affinity of the polypeptide encoded by the KRAS missense mutation to RAF effector protein; and (iv) generating, by the one or more processors, based on the conservation metric, the pro-oncogenic advantage metric, and the immunogenic cost metric, a fitness score, wherein generating the fitness score comprises assigning weights to the conservation metric, the pro-oncogenic advantage metric, and the immunogenic cost metric, and applying a divergence-based statistical analysis to optimize the conservation metric, the pro-oncogenic advantage metric, and the immunogenic cost metric; and (c) identifying, by the one or more processors, a subset of KRAS missense mutations that have fitness scores that exceed a threshold as high-fitness KRAS mutations and/or identifying, by the one or more processors, a subset of KRAS missense mutations that have fitness scores that fall below a threshold as low-fitness KRAS mutations.

In another aspect, the present disclosure provides one or more computing devices comprising one or more processors and a computer-readable storage medium with instructions executable by the one or more processors to cause the computing device to perform steps for predicting fitness of KRAS mutations, said steps comprising: (a) obtaining, by one or more processors of a computing device, a dataset comprising a plurality of KRAS missense mutations present in one or more subjects; (b) for each KRAS missense mutation in the dataset, applying a model to obtain a fitness score, wherein the model comprises: (i) generating, by the one or more processors, a conservation metric for the KRAS missense mutation based on an evolutionary rate assigned to each amino acid of a polypeptide encoded by the KRAS missense mutation; (ii) generating, by the one or more processors, an immunogenic cost metric for the KRAS missense mutation based on binding affinities of MHC class I molecules to KRAS-derived nonamer neopeptides including the KRAS missense mutation; (iii) generating, by the one or more processors, a pro-oncogenic advantage metric for the KRAS missense mutation based on binding affinity of the polypeptide encoded by the KRAS missense mutation to RAF effector protein; and (iv) generating, by the one or more processors, based on the conservation metric, the pro-oncogenic advantage metric, and the immunogenic cost metric, a fitness score, wherein generating the fitness score comprises assigning weights to the conservation metric, the pro-oncogenic advantage metric, and the immunogenic cost metric, and applying a divergence-based statistical analysis to optimize the conservation metric, the pro-oncogenic advantage metric, and the immunogenic cost metric; and (c) identifying, by the one or more processors, a subset of KRAS missense mutations that have fitness scores that exceed a threshold as high-fitness KRAS mutations and/or identifying, by the one or more processors, a subset of KRAS missense mutations that have fitness scores that fall below a threshold as low-fitness KRAS mutations.

### Therapeutic Selection Methods of the Present Technology

In one aspect, the present disclosure provides a method for selecting a patient diagnosed with or at risk for cancer for treatment with an immune checkpoint inhibitor comprising: detecting the presence of a p53 mutation in a biological sample obtained from the patient, wherein the p53 mutation is selected from the group consisting of R248Q, R273H, R248W, R273C, and G245S; and administering to the patient an effective amount of the immune checkpoint inhibitor. The p53 mutation may be a germline or somatic mutation. Examples of cancers, include but are not limited to, colorectal cancer, lung cancer, breast cancer, ovarian cancer, uterine cancer, or thyroid cancer. Additionally or alternatively, in some embodiments, the the p53 mutation is detected via in situ hybridization, polymerase chain reaction (PCR), Next-generation sequencing, Northern blotting, microarray, dot or slot blots, fluorescent in situ hybridization (FISH), electrophoresis, chromatography, or mass spectroscopy. In certain embodiments, the biological sample comprises blood, plasma, serum or tissue.

Examples of immune checkpoint inhibitors include, but are not limited to, an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody.

Nucleic Acid Amplification and/or Detection

Polynucleotides encoding p53 can be detected by the use of nucleic acid amplification techniques that are well known in the art. The starting material may be cDNA, RNA or mRNA. Nucleic acid amplification can be linear or exponential. Target sequences may be detected by the use of amplification methods with the aid of oligonucleotide primers or probes designed to interact with or hybridize to a particular target sequence in a specific manner, thus amplifying the target sequence.

Non-limiting examples of nucleic acid amplification techniques include reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7(suppl 2):S 11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80: 1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120),7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes can be designed so that they hybridize under stringent conditions to p53 target nucleic acid sequences in humans. In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, for example, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### EXAMPLES

The present technology is further illustrated by the following Examples, which should not be construed as limiting in any way.

### Example 1: Materials and Methods

### Background Distribution Determinants

Predicted background mutation frequencies. We quantified the background mutation frequencies of amino acid mutations across commonly mutated tumor suppressors and oncogenes with dinucleotide-based mutation rates³⁸. In brief, dinucleotide-based mutation rates were derived from a nonreversible mutation model based on alignments between human and mouse non-coding DNA sequences from human chromosomes 10 and 21. In this mutational model, there are no external mutational pressures such as those derived from UV radiation or toxins such as aflatoxin. Since we observed such strong mutation distribution conservation across databases, we posited that although such mutational signatures may be relevant, intrinsic mutational processes should be the main determinant of background mutation rates. The internal CpG-associated C→T mutation signature is an order of magnitude more common than the other types of mutations. For each nucleotide position across each gene, we assign a mutational rate which is the average of the left and and right neighboring dinucleotide mutation rates for dinucleotides containing the position in question, effectively assigning a trinucleotide mutation rate for single nucleotide mutations. To derive the background mutation frequencies, we first assign each nucleotide in the coding region of each gene an effective trinucleotide mutation rate. Next, for each amino acid mutation we sum the mutation rates of all of its possible nucleotide mutations. Finally, we normalize the rates to create a probability distribution.

Let lₙ and rₙ be the rates of mutation of a nucleotide n corresponding to its left and and right dinucleotides. Further, let the set Nₘ correspond to all of the nucleotide mutations that result in amino acid mutation m. We set the average rate of mutation for a nucleotide n as μₙ=(lₙ+rₙ)/2. We set the background mutation frequency of an amino acid mutation by normalizing across all amino acid mutation rates,

$${p}_{m} = \frac{{\sum }_{n ∈ {N}_{m}} {μ}_{n}}{{\sum }_{{m}^{′}} {\sum }_{{n}^{′} ∈ {N}_{{m}^{′}}} {μ}_{{n}^{′}}} . \qquad ( 1 )$$

We consider the full gene sequence with both introns and exons, which we downloaded from the National Center for Biotechnology Information (NCBI)³⁹. All nucleotides within the coding region of each gene have a right and left neighboring nucleotide, i.e. there are no boundary cases. The amino acid alterations corresponding to each nucleotide mutation were determined with the SnpEff software package (Version 4.3)⁴⁰.

### Inference of Apparent Dimer Dissociation Constants

In order to estimate the functional capacity of all possible p53 missense mutants, we leveraged a trans-activation yeast assay dataset⁵. In that work, all possible p53 missense mutations derived from single nucleotide mutations were mono-alleleically expressed in yeast cells in which eight target promoter sequences were tagged with either enhanced green fluorescent protein (EGFP) on the p21^{WAF1} target sequence or Ds-Red on the other seven target genes (MDM2, BAX, GADD45, h1433s, p53AIP1, NOXA, and p53R2). The p21^{WAF1} and MDM2 sequences are human-derived, and the others were synthetic with p53 response elements. Fluorescence intensity was measured for each mutant. The average relative fluorescence intensity of each p53 mutant was reported with respect to wild-type p53.

Under such conditions, it is assumed that all of the mutant and wild-type p53 proteins are expressed at equally-low concentration. Therefore, we expect the relative transactivation values reported are largely driven by the different affinities of each mutant to the target DNA sequence. p53 monomers have a tendency to oligomerize as dimers or tetramers⁴¹. p53 primarily transactivates target DNA in a highly-cooperative manner as a tetramer, i.e. a “dimer of dimers”⁴², which may sequentially bind the same promoter sequence. The affinity of the second dimer is typically much larger than the affinity of the first dimer due to cooperativity. The “effective dimer dissociation constant” is equal to the geometric mean of the two dimer dissociation constants. The effective dimer dissociation constants of truncated wild-type p53 (DNA-binding domain and oligomerization domain, amino acids 94-360) to well-known targets of p53 transactivation have also been quantified in vitro⁴³. For promoter sites with multiple binding sites, we take the geometric mean of the affinities as the effective affinity. Additionally, it has been shown that the N- and C-termini of p53 regulate DNA binding, as they non-specifically bind to DNA and reduce the effective affinity of the dimer complex to a specific sequence, with an approximately 10-fold reduction in specific binding affinity both in vitro and in vivo⁴⁴⁻⁴⁶. Furthermore, the termini contain residues that are targets of post-translational modification such as acetylation⁴⁷ which may or may not be post-translationally modified. Therefore, we correct for the full-sequence dissociation constant by multiplying the reported dissociation constants by a factor of 10 in order to correct for the termini.

The likelihood of p53 binding a target sequence will involve both the p53 concentration and the amino acid sequence-based binding affinity. We interpret the probability that p53 binds target DNA via a Hill function with cooperativity of 2⁴²:

$${P}_{m}^{DNA} = \frac{{( {L}_{REF} )}^{2}}{{( {L}_{REF} )}^{2} + {( {K}_{T}^{m} )}^{2}} , \qquad ( 2 )$$

where Pₘ^{DNA} is the probability of binding target DNA, L_{REF} is the concentration of p53 dimer in the yeast assay, and K_{T}^{m} is the effective dimer dissociation constant specific for a DNA promoter sequence.

The yeast assays report an averaged relative transactivation value, which is the ratio of the mutant fluorescence over the wild-type fluorescence. We assume the fluorescence value is in the linear range of the binding curve, so that the fluorescence of wild-type or mutant p53 binding DNA is proportional to its probability of binding DNA. The relative transactivation value can be estimated as:

$$\frac{{F}_{m}}{\vert {F}_{wt}} = \frac{{( {L}_{REF} )}^{2} + {( {K}_{T}^{w t} )}^{2}}{{( {L}_{REF} )}^{2} + {( {K}_{T}^{m} )}^{2}} , \qquad ( 3 )$$

where Fₘ and F_{wt} are the mutant and wild-type fluorescence values, and

$${K}_{T}^{w t} and {K}_{T}^{m}$$

are the effective dimer dissociation constants for the wild-type homotetramer and the mutant homotetramer for a specific DNA target sequence. Therefore, we can transform the mutant-specific relative transactivation to the mutant-specific K_{T}^{m} via:

$${K}_{T}^{m} = \sqrt{\frac{{( {K}_{T}^{w t} )}^{2} + {( {L}_{REF} )}^{2} ( 1 - ( {F}_{m} / {F}_{w t} ) )}{( {F}_{m} / {F}_{w t} )}} ≃ \frac{{K}_{T}^{w t}}{\sqrt{( {F}_{m} / {F}_{w t} )}} , \qquad ( 4 )$$

where the final approximation arises since the dissociation constants tend to be of order ≥10² nM, as the ratio of flourescence values is bounded in the experimental data by 0≤(Fₘ/F_{wt})≤4.6. We choose a reference concentration for a p53 dimer L_{REF}, of approximately 1 nM that is consistent with a previously-defined low-concentration regime of p53 in yeast⁴⁸. To account for non-specific binding, we add an offset of 9×10⁻⁴, which is an order of magnitude lower than the lowest non-zero transactivation value in the experiment (Fₘ/F_{wt}=0.001).

### Inference of Tissue- and Mutant-Specific Concentrations

Under normal conditions, wild-type p53 is maintained at low concentrations and has a half-life of approximately 20 minutes largely via a negative-feedback loop with MDM2^{41,50}. Under conditions of stress, wild-type concentration typically rises, increasing transactivation of target genes responsible for cellular stress response. Missense-derived mutant p53 concentration tends to increase to non-typically high levels that are both tissue- and mutant-specific, while nonsense mutations tend to strongly reduce p53 concentration¹²⁻¹⁴.

The fitness model strongly depends on the mutant concentration, as it links both the functional and immune components via a biophysical binding model. However, quantitative concentration information for most p53 mutants is unavailable. To address this, we aimed to infer each missense mutant's concentration. p53 concentration is directly regulated by MDM2 and p53 mutants alter the ability for the transcription factor to bind promoter sites on DNA, such as the MDM2 promoter site. From this, we expect that mutants which retain MDM2 promoter DNA capacity will induce wild-type p53 comparable levels of MDM2, which will in turn constrain p53 concentration to wild-type levels. Mutants which greatly reduce p53 binding of MDM2 promoter DNA will reduce the amount of circulating MDM2, thus permitting a higher concentration of mutant p53. We leverage this principle and apply it to a TCGA proteomics dataset to infer tissue- and mutant-specific concentrations utilizing inferred mutant DNA-binding affinities from previous work in yeast⁵. In doing so we quantify the role of the p53-MDM2 negative feedback loop in a large dataset such as TCGA. We describe the methods in detail below.

Quantifying mutant p53 concentrations. For a particular sample, the concentration of p53 will depend on the tissue type. It will also depend on the mutational status and the number of mutant/wild-type p53 alleles available. It will also strongly depend on tumor heterogeneity, such as the clonal status of the mutation and the purity of the sample. We aimed to quantify the distribution of mutant p53 concentration using TCGA, where the Reverse-Phase Protein Assay (RPPA) has been used to quantify relative protein expression in TCGA samples. We downloaded Level 4 RPPA data (TCGA-PANCAN32-L4) from The Cancer Proteome Atlas (TCPA)^{51,52}. In a manner similar to a Western blot, protein expression is inferred via fluorescence from a tagged antibody. To account for batch effects, the inferred log₂ concentration values are median-normalized by subtracting each sample's value with two medians—the median log₂ concentration for that protein across all samples and the median log₂ concentration of all of the proteins in one sample. The value reported is proportional to the log₂ of the true concentration in the sample. If we define RPPA reported values as R, the subtracted constants as c and assume that c is distributed around a central value, and the protein concentration as L, then R=log₂L−c or L=C×2^{R}, where C is a constant which provides the appropriate units. We show the values for C, and by extension, c, are distributed around a central value for wild-type p53.

Multiple efforts have tried to quantify wild-type p53 concentration in cells under different conditions,^{41,50} typically converging on concentrations on the order of 10² to 10³ nM across different cell types. In MCF-7 wild-type p53 breast cancer cell lines, the average concentration is estimated at approximately 150 nM⁵⁰. We leverage this value to define the constant C, with appropriate nanomolar units (nM) using TCGA wild-type breast cancer (BRCA) p53 RPPA data. An average concentration of 150 nM of p53 in MCF-7 breast cancer cells with two wild-type p53 alleles means that we expect each p53 allele to contribute approximately 75 nM. In order to find the equivalent protein expression in the RPPA dataset, we examined the distribution of 2^{R} per allele in p53 wild-type breast cancer cells (BRCA) in TCGA. We selected samples for which there were no p53 mutations nor amplifications/deletions. In general, for each p53-mutated TCGA sample, when possible, wd (1) estimate the purity of the sample, (2) estimate the clonal status of the p53 mutation in the tumor, (3) infer the number of p53 alleles, and (4) distinguish which p53 alleles are wild-type and mutant. This methodology is described below.

Estimating tumor heterogeneity. The p53 RPPA value for a mutant p53 tumor sample in TCGA is not entirely due to the mutated p53. The p53 RPPA value of a tumor sample may be decomposed as:

$${2}^{{R}_{s}} = {2}^{{R}_{w t}} [ {𝒞}_{N} ( 1 - p ) + {𝒞}_{T} p ( 1 - f ) + ( {𝒞}_{T} - {N}_{m} ) pf ] + {2}^{{R}_{m}} [ {N}_{m} pf ] , \qquad ( 5 )$$

where R_{S} is the sample p53 RPPA value, R_{wt} is the wild-type p53 component of the sample RPPA, R_{M} is the mutant p53 component of the sample RPPA, C_{N} is the expected p53 ploidy in typical, non-cancerous cells, p is the purity of the sample, f is the cancer cell fraction, C_{T} is the number of p53 alleles in tumor cells, and Nₘ is the number of mutant alleles in a p53-mutant cell. The components may be justified as follows:
WT p53 alleles from the normal portion of the sample: C_{N}(1−p)
WT p53 alleles from the tumor portion of the sample without p53 mutations:

$${C}_{τ} p ( 1 - f )$$

WT p53 alleles from the tumor portion of the sample with p53 mutations:

$$( {C}_{T} - {N}_{m} ) pf$$

MT p53 alleles from the tumor portion of sample with p53 mutations: Nₘpf subject to the following constraints: C_{N}=2 (typical p53 ploidy), C_{T}≥0,Nₘ≥0, 0≤Nₘ≤C_{T}, 0≤p≤1, and 0≤f≤1. For example, if C_{T}=2 and Nₘ=1, then the cell is heterozygous in mutant p53, and if Nₘ=2, then it is homozygous in mutant p53.

The purity of TCGA samples was quantified with ASCAT⁵³ and downloaded from COSMIC⁵⁴. Copy number variation data for TCGA samples was downloaded from the National Cancer Institute's Genomic Data Commons repository⁵⁵. For processing of p53 copy number variation data, we averaged all p53 copy number values, after converting from segmentation values (C_{T}=2×2^{seg}, where seg is the segmentation value), overlapping with the TP53 gene region (defined as chr17: [7661779, 7687550], reference genome GRCh38). Neither the cancer cell fraction nor the zygosity of p53 mutants in TCGA have been previously quantified, which we compute in the next section. Knowing these quantities allows us to solve for 2^{R}^{_{M}}, a value that is proportional to the concentration of one mutant p53 allele.

### Estimating Cancer Cell Fraction f and the Number of Mutant Alleles Nₘ.

Sequencing of DNA from tumor samples provides the number of reads that cover a mutation. It indicates the number of reference and alternate alleles, given a reference genome. If we define the number of reference allele reads as Rᵣ, the number of alternate allele reads to Rₐ, and the variant allele fraction as V, we have:

$$V = \frac{{R}_{a}}{{R}_{r} + {R}_{a}} , \qquad ( 6 )$$

where 0≤V≤1. The variant allele fractions for p53 mutations in TCGA were downloaded from the Genomic Data Commons repository,⁵⁵ the values of which were averaged across mutation callers. Theoretically, the variant allele frequency can also be defined as⁵⁶⁻⁵⁹:

$$V = \frac{{pfN}_{m}}{{𝒞}_{𝒩} ( 1 - p ) + {𝒞}_{T} p} . \qquad ( 7 )$$

We have estimates for all variables except f and N_{M}. The term w=fN_{M} is defined as the multiplicity. The probability distribution of the number of reads that align to a mutation may be interpreted in terms of a binomial distribution, where Rₐ is the number of successes and Rᵣ+Rₐ is the number of trials. We find the value of w that maximizes the posterior distribution. We treat f and Nₘ as independent. We calculate f by computing the value that maximizes the likelihood of getting a sample variant allele frequency according to the following procedure:

For each sample in TCGA we obtain a value of Rᵣ, Rₐ, and V for a p53 mutant.

We vary f from 0.01 to 1 for 100 evenly-spaced values and calculate V across the variations of f.

We calculate the probability of getting Rₐ successes given Rᵣ+Rₐ trials given a probability V for each varied

$$f . p = B ( {R}_{r} + {R}_{a} , V ) = ( \begin{matrix}{R}_{r} + {R}_{a} \\ {R}_{a}\end{matrix} ) {{V}^{{R}_{a}} ( 1 - V )}^{{R}_{r}} .$$

We then normalize the probability distribution and find the cancer cell fraction that maximizes the binomial probability. This is fₒₚₜ.

Finally, we solve for Nₘ using the actual V from TCGA sample and round it to an integer. If Nₘ>C_{T}, then we set Nₘ=C_{T}.

Having these components for TCGA tumors, we can estimate the heterozygosity of p53 mutations, the concentration of the mutant alleles in a sample, and the typical concentrations of different p53 mutants. Furthermore, we can quantify the MDM2 and p53 negative-feedback loop from such data as a check for consistency. There is no RPPA information available for the MDM2 protein, but there is RNA expression data available. As MDM2 is transactivated by p53, we expect MDM2 RNA expression (quantified in Transcripts Per Million (TPM)) to be proportional to MDM2 concentration, and negatively related to p53 concentration. Similarly, we expect the p53 concentration to be positively correlated with p53 RNA expression as a check on self-consistency.

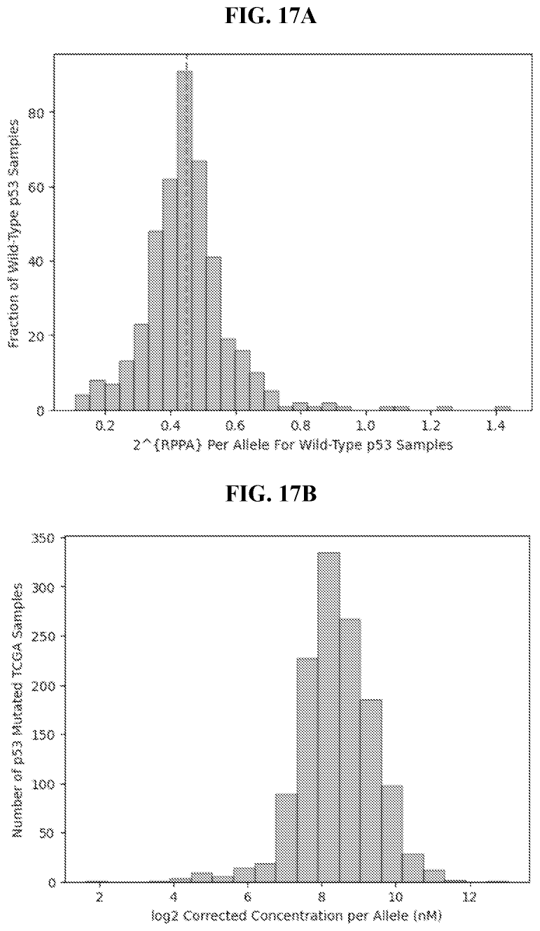

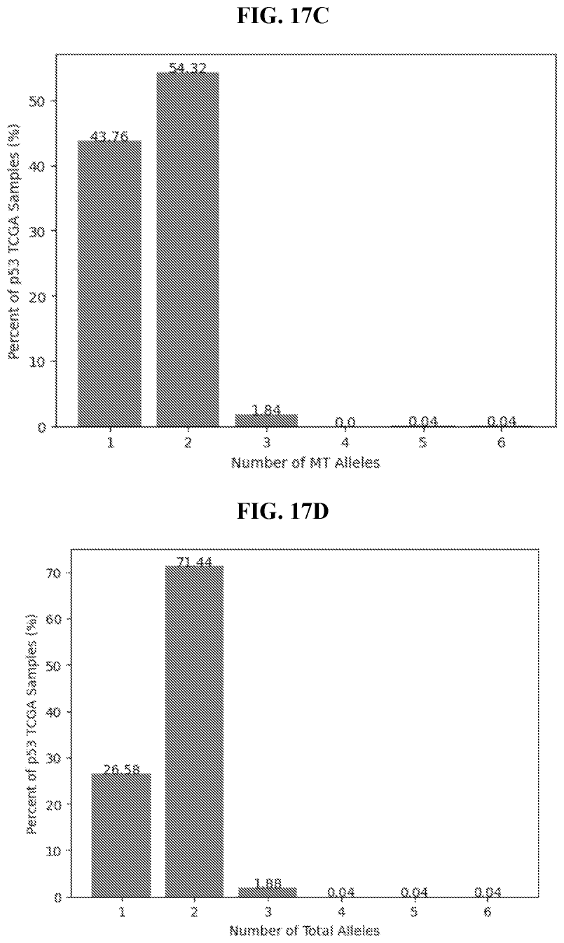

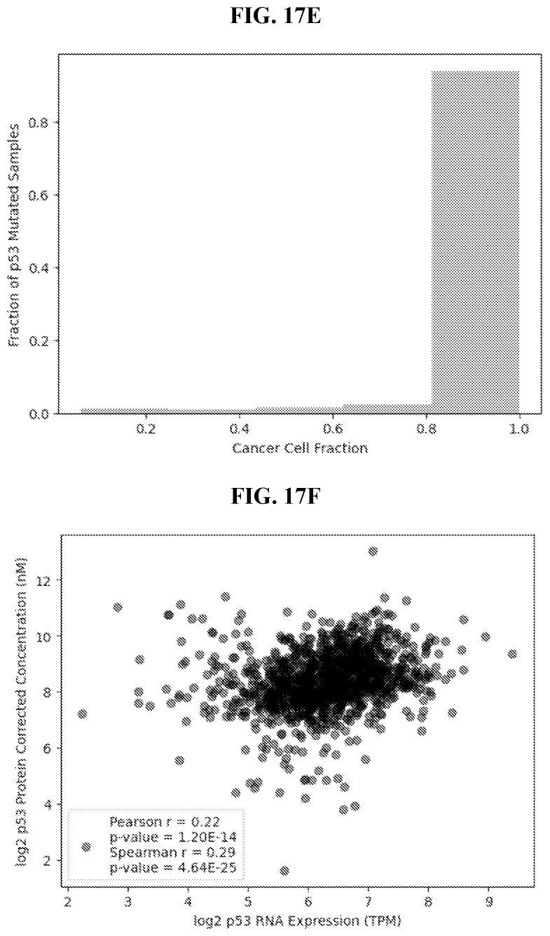

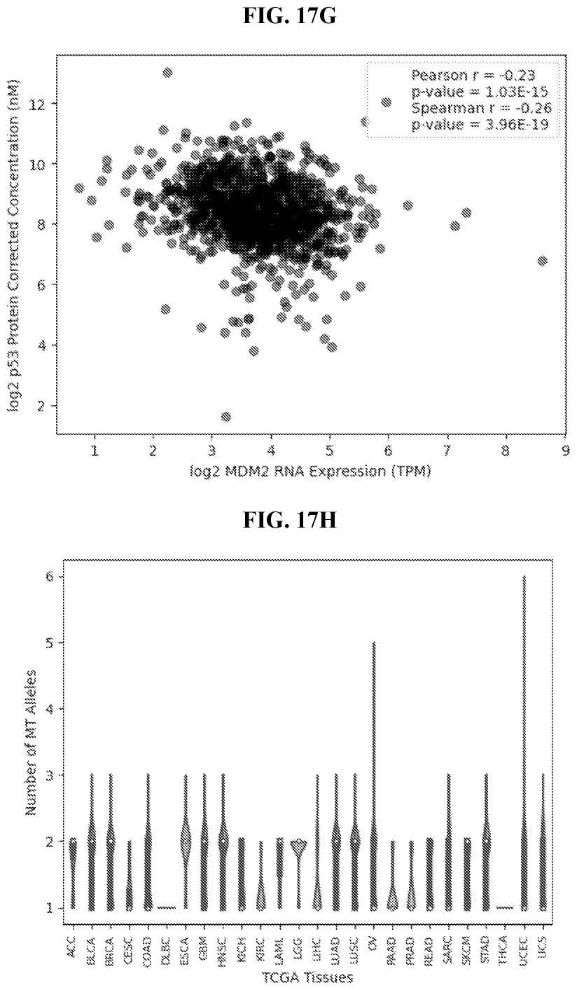

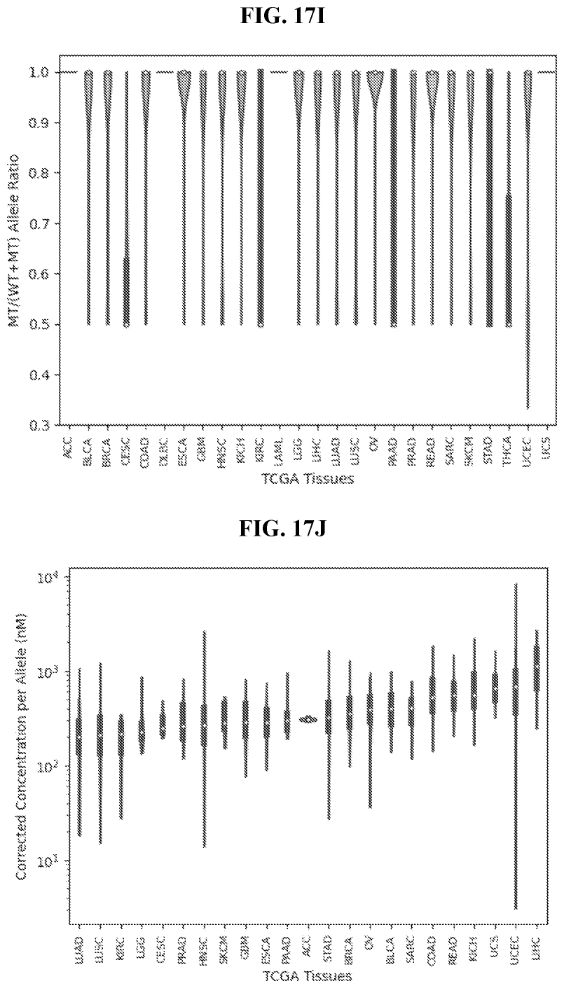

Inferred heterogeneity and related transcriptomic and proteomic analysis for p53 mutations in TCGA is presented in FIG. 17.

Computing the effective p53 MDM2 promoter affinity in TCGA samples. By normalizing by the number of mutant p53 alleles, the methods outlined above allow us to infer the per-allele mutant concentration in a particular sample. Next, we predicted the level of MDM2 transactivation within a cancer cell based on the estimated distribution of mutant and wild-type p53 alleles. Samples in TCGA may contain different distributions of the number of MT and WT alleles, as some may be heterozygous in a p53 mutation, others may be homozygous, and others may have deletions/amplifications in the TP53 gene. A sample with both wild-type and mutant TP53 alleles will not only contain fully mutant and fully wild-type tetramers, but to a larger extent will also contain a distribution of hybrid wild-type and mutant tetramers. Previous work has attempted to quantify the effect that mixed mutant and wild-type tetramers have on binding affinity, suggesting that hybrid wild-type and mutant p53 tetramers are not fully inactivated, taking approximately three mutant p53 monomer subunits to truly render a p53 tetramer non-functional for certain mutations⁶⁰.

The dissociation constant used in the cooperative Hill function for the functional term is the apparent dimer dissociation constant, defined as the geometric mean of the sequential dissociation constants of two dimers to same promoter region, where the first is large and the second is small⁴². We can infer the wild-type dimer dissociation constant from previous work⁴³, and we earlier estimated the dimer dissociation constant for a fully-mutant p53 tetramer. In order to estimate the effective dissociation constants associated with mixed wild-type/mutant p53 tetramers, we assume that an equally-mixed tetramer composed of one WT:WT dimer and one MT:MT dimer must have the same binding efficiency as one composed of two WT:MT dimers. The dimer dissociation constant of an equally-mixed tetramer is assumed to be

$${K}_{T}^{MT , WT} = \sqrt{{K}_{T}^{2 WT} \times {K}_{T}^{2 MT}} .$$

By similar logic, the dimer dissociation constant of a 3 WT:1 MT mixed tetramer is assumed to be

$$\sqrt{{K}_{T}^{2 WT} \times {K}_{T}^{MT , WT}} ,$$

and the dimer dissociation constant of a 1 WT: 3 MT mixed tetramer is assumed to be

$$\sqrt{{K}_{T}^{2 MT} \times {K}_{T}^{MT , WT}} .$$

The probability of a particular tetramer species existing will depend on the number of WT and MT TP53 alleles in a sample. If we define the total number of TP53 alleles as N_{Tot}=C_{N}+C_{T}, the number of TP53 wild-type alleles as C_{N}, and the number of TP53 mutant alleles as C_{T}, then the probability of a wild-type monomer incorporated into a tetramer is q_{w}=C_{N}/N_{Tot}, and the corresponding mutant probability is qₘ=C_{T}/N_{Tot}.

The probability of a tetramer λ is then:

$${P}_{λ} = P ( X , Y = 4 - X ) = ( \begin{matrix}{N}_{Tot}^{X} \\ X\end{matrix} ) {q}_{w}^{X} {q}_{m}^{Y} , \qquad ( 8 )$$

where X is the number of wild-type monomer units in the tetramer, Y is the number of mutant monomer units in the tetramer, and X+Y=4 for all tetramers λϵΛ.

We define the effective association constant of MDM2 promoter as the expectation value across tetramer species, weighted by their probability of being formed in a cell:

$${〈 \frac{1}{{K}_{T}} 〉}_{Λ} = {\sum }_{λ ∈ Λ} {P}_{λ} \frac{1}{{K}_{T}^{λ}} = ( \begin{matrix}{N}_{tot} \\ X\end{matrix} ) {q}_{w}^{X} {q}_{m}^{Y} \frac{1}{{K}_{T}^{λ}} . \qquad ( 9 )$$

Now we can determine if there are any relationships between the effective MDM2 promoter association constant and the per-allele corrected concentration across TCGA samples with available data. In pan-cancer and tissue-specific settings, we plot all of the unique MDM2 promoter association constants versus the median of the per-allele concentrations corresponding to that association constant to control for noise. We fit a line to the data using a least-squares regression, which defines a quantitative expression for the relationship between normalized mutant p53 concentration and expected MDM2 transactivation by p53 across missense mutants. The predominantly negative relationships between the expected MDM2 association constant and the p53 concentration provide additional evidence for the p53-MDM2 negative feedback loop in TCGA and allow us to estimate tissue- and mutant-specific concentrations based on the regression line for mutations with unavailable concentration data.

In all cases, the relationship between the effective MDM2 promoter association constant and p53 concentration is given by the expression:

$${log}_{2} {L}_{p}^{m} = a \times {〈 \frac{1}{{K}_{T}} 〉}_{Λ} + b , \qquad ( 10 )$$

where

$${log}_{2} {L}_{p}^{m}$$

is the log₂ of the per-allele concentration of mutant p53 monomers,

$${〈 \frac{1}{{K}_{T}} 〉}_{Λ}$$

is the effective association constant of MDM2 promoter across the Λ tetramers, and a and b are the slope and intercept that are being fit. We present fitness models in both the pan-cancer setting and a tissue specific model for colorectal cancer. In the pan-cancer setting, a=−133.06 and b=8.68.

### Fitness Model

Fitness model components. We propose a minimal biophysical model of the fitness advantage a tumour acquires from a TP53 mutation in order to explain the observed population mutation frequency distribution. We expect higher fitness TP53 mutations are more likely to be fixed in tumors and therefore will have a higher observed mutation frequency, and the opposite will occur for less fit mutations.

The relative fitness of a mutation m for a patient with HLA haplotypes H=[A₁,A₂,B₁,B₂,C₁,C₂] is defined as:

$${f}_{m} ( H ) = {σ}_{T} {T}_{m} + {σ}_{I} {I}_{m} ( H ) , \qquad ( 11 )$$

where the term σ_{T}Tₘ quantifies the effect a TP53 mutation has on mutant p53 transcription factor-associated binding activity, and σₗIₘ(H) corresponds to the immunogenicity of the mutant peptides corresponding to a p53 mutation, which will depend on the set of HLA-I molecules in haplotype H. The parameters {σ_{T},σ₁} assign relative weights to the fitness components and set the overall scale of the fitness amplitude. They are optimized to fit the the training set in our model.

We define Tₘ as the median probability that a mutant p53 homotetramer does not bind target promoter sites in DNA across the eight target genes (WAF1, MDM2, BAX, h1433s, AIP1, GADD45, NOXA, and P53R2) for which we have data available from previous work defining mutant p53 binding in a quantitative yeast assay⁵. Tₘ is modeled by a cooperative Hill function with a cooperativity coefficient of two,⁴²

$${T}_{m} = \frac{{( {K}_{T}^{m} )}^{2}}{{( {L}_{D}^{{m}_{}} )}^{2} + {( {K}_{T}^{m} )}^{2}} , \qquad ( 12 )$$

where

$${L}_{D}^{m}$$

is the concentration of a mutant p53 homodimer for mutation m, which is equivalent to half of the total mutant p53 monomer concentration, and

$${K}_{T}^{m}$$

is the median apparent dimer dissociation constant for binding target DNA across the eight target genes studied for mutation m. The methods for computation of

$${K}_{T}^{m} and {L}_{D}^{m}$$

are described in detail in the subsequent sections.

We define Iₘ(H) as the geometric mean of the predicted probabilities of all mutant peptides binding class-I MHC molecules for mutation m, via a non-cooperative Hill function,

$${I}_{m} ( H ) = {( {\prod }_{p ∈ {P}_{m} , h ∈ H} \frac{{L}_{p}^{m}}{{L}_{p}^{m} + {K}_{l}^{m} ( p , h )} )}^{\frac{1}{[ {P}_{m} ] \times [ H ]}} , \qquad ( 13 )$$

where p is a peptide, Pₘ is the set of mutated peptides around mutation m, h is an HLA-I within the set H of germline HLA-I in the host,

$${L}_{p}^{m}$$

is the concentration of the peptide (which is also the p53 monomer concentration and twice the p53 dimer concentration), and Kₗ^{m}(p, h) is the predicted dissociation constant between a mutant peptide and an HLA-I molecule. Here we consider all mutated peptides of length nine, the most common length presented⁶¹. Typically, |Pₘ|=9 and |H|=6; exceptions may occur if the mutation occurs close to the edges of the protein or if there are additional mutations which reduce MHC-I expression. The peptides are derived from canonical protein transcripts as determined by UniProt⁶².

We infer the concentrations

$${L}_{p}^{m} and {L}_{D}^{m} = {L}_{p}^{m} / 2$$

from TCGA in nanomolar (nM) units, and infer dissociation constants

$${K}_{I}^{m} ( p , h )$$

in nM units from computationally-derived IC56 values calculated from NetMHC 3.4 and NetMHC 4.0^{61,63-65}. We estimated the effective mutant peptide MHC-I affinities K_{eff}_{₁}^{m}(p, h) for all TP53 missense mutants by computing:

$$\frac{1}{{K}_{{eff}_{I}^{m}}} = {〈 {max}_{{p ∈ P}_{h ∈ H}} \frac{1}{{K}_{l}^{m} ( p , h )} 〉}_{H ∈ {D}_{H}} , \qquad ( 14 )$$

with all mutant peptides p∈Pₘ and HLA-I h∈H across the population D_{H}. We also consider alternative fitness models with additional components, which we discuss in the section on model performance and comparison.

Let mutation m occur in a patient with MHC-I haplotype H. The relative contribution of a mutation to the growth of the tumor clone with this mutation is described by exp[(f_{wt}+fₘ(H; σ_{T},σ_{I}))], where f_{wt} is the background growth rate of the tumor clone without the mutation and fₘ is the fitness effect of mutation m. Given all possible mutations and their background frequencies, pₘ, as determined by the background mutation rates, the model-predicted frequency of an observed mutation m within the haplotype H is given by

$${\hat{x}}_{m} ( H ) = {Z}_{H}^{- 1} {p}_{m} exp [ {f}_{m} ( H ) ] , \qquad ( 15 )$$

where Z_{H}=Σₘpₘ exp[fₘ(H)]. Since we consider the relative frequencies of mutations, the constant wildtype growth term f_{wt} factors out from the above expression. The population level predictions for frequencies of mutations are computed as the expectation value over the database of haplotypes D_{H} representative of a population,

$${\hat{x}}_{m} = {〈 {\hat{x}}_{m} ( H ) 〉}_{H ∈ {D}_{H}} . \qquad ( 16 )$$

The mutation frequency predictions depend on the fitness model parameters: \hat{x}ₘ≡\hat{x}ₘ(σ_{T},σ_{I}). Each mutation occurs within a TP53 codon. We define the codon mutation frequency as the sum of the missense mutation frequencies that alter a codon's amino acid (i.e. all missense mutations within a codon). For instance, the codon frequency at position R175 is the sum of all individual missense mutations which alter the arginine corresponding to codon 175. This step is done as an additional check on the predictive power of the fitness model, as the p53 mutation hotspots are clustered in a set of well-defined hotspot codons.

The relative fitness of a TP53 mutation defines whether or not its population frequency increases or decreases with respect to the background mutation frequency. Higher fitness mutations will increase their population frequency with respect to their background mutation frequency, and lower fitness mutants will have a lower population frequency with respect to their background mutation frequency. We define the predicted ratio Ŵₘ=\hat{x}ₘ/pₘ, as the relative increase or decrease of the predicted frequency with respect to the background mutation frequency for mutation m, and the posterior ratio Wₘ=xₘ/pₘ as the relative increase or decrease of the observed mutation frequency with respect to the background mutation frequency for mutation m. These terms are the Wrightian fitness of a mutation—ratios which are >1 indicate population frequency growth, and ratios which are <1 indicate population frequency decrease.

Haplotype distributions. We train the weights for our model on the missense p53 mutation frequencies and haplotypes available within TCGA⁶⁶. Our training cohort was chosen since these are non-simulated haplotypes with full MHC-I linkage information. There are a total of 8,507 haplotypes which correspond to 6,379 unique haplotypes. For testing the relevance of the sampled haplotype space on the fitness model predictions, we used marginal haplotype frequencies from the National Marrow Donor Program (NMDP) database corresponding to European Caucasian-Americans, which provides information on 1,242,890 donors^{67,68}. Within this database, there is no extensive haplotype information but there is extensive individual HLA population frequency information.

We assume the MHC-I haplotype will consist of two each of HLA-A, HLA-B, and HLA-C for a total of 6 MHC-I genes. We assume a multinomial distribution with an independent frequency model without MHC-I linkage for each HLA-A, HLA-B, and HLA-C gene. We constructed all possible haplotypes using all available MHC-I within the database. The number of heterozygous HLA-I genes is given by N_{H}, where N_{H}∈[0, 1, 2, 3]. The probability of a haplotype H=[A₁,A₂,B₁,B₂,C₁,C₂] is given by

$$p ( H ) = {2}^{{N}_{H}} {\prod }_{h ∈ H} {p}_{h} , \qquad ( 17 )$$

where pₕ corresponds to the marginal probability of HLA-I h within haplotype H.

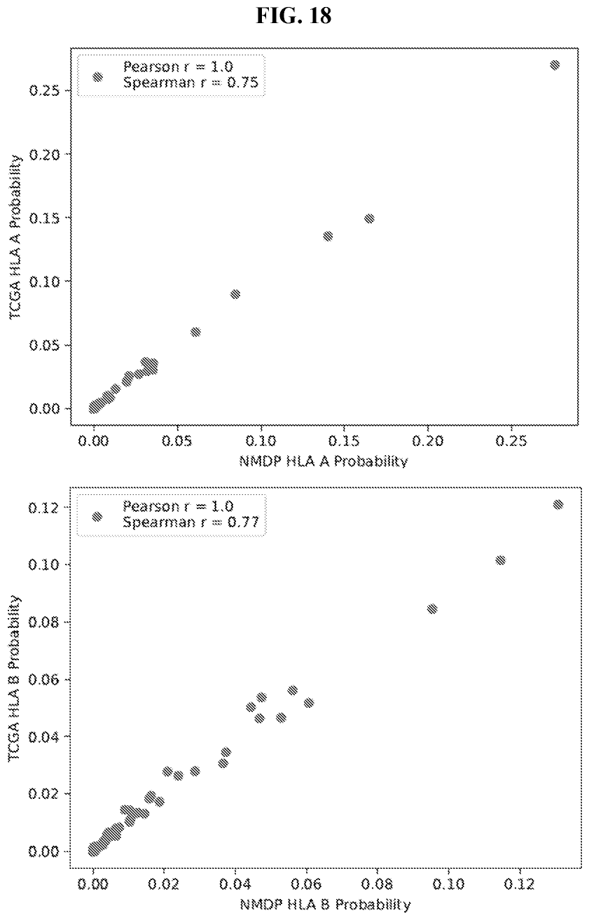

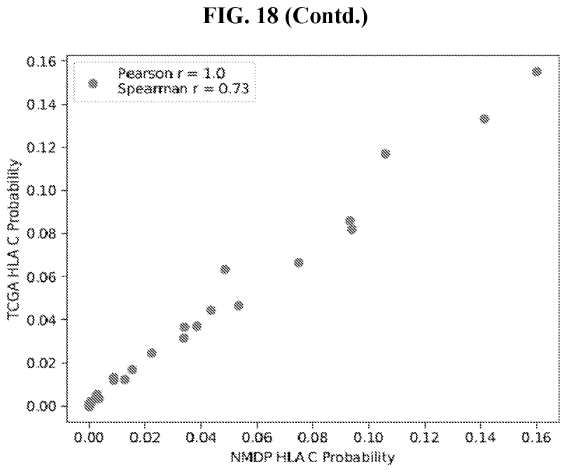

We sort the haplotype probabilities and take a subset of the most frequent haplotypes. In both training and testing, we compute the expected mutation frequency for each haplotype and calculate a weighted average across the population, with weights given by the expected haplotype probabilities, resulting in the expected mutation population frequency according to Eq. 16. TCGA and NMDP-derived simulated HLA-I frequencies are highly correlated (FIG. 18).

### Model Training

Model fitting. To optimize the fitness model parameters, Θ={σ_{T},σ_{I}}, we minimize the cross entropy between the observed mutation frequencies xₘ and the frequencies predicted by the model, \hat{x}ₘ,

$$ℋ ( {x}_{m} , {\hat{x}}_{m} , ; ⊖ ) = - {\sum }_{m} {x}_{m} log {\hat{x}}_{m} ( ⊖ ) . \qquad ( 18 )$$

Minimization of the cross-entropy is equivalent to the minimization of the Kullback-Leibler divergence between the distributions of the observed and predicted frequencies and to maximization of the likelihood of the mutation data under the given fitness model. Each unique observed mutation m in the database Dₘ is predicted to occur with probability \hat{x}ₘ(Θ). The data log-likelihood under our model is given by:

$$ℒ ( {D}_{m} \vert ⊖ ) = {\sum }_{m ∈ {D}_{m}} log {\hat{x}}_{m} ( ⊖ ) = - n ℋ ( ⊖ ) , \qquad ( 19 )$$

where n=|Dₘ| is the size of the database of p53 mutations. We minimize the cross entropy using the limited-memory Broyden-Fletcher-Goldfarb-Shanno algorithm (L-BFGS) with the analytically-computed gradient of the cross-entropy. We find that the optimized parameters for pan-cancer TCGA are

$${σ}_{T}^{*} = 4.152 and {σ}_{I}^{*} = - 42.212 .$$

Alternative models. We compared our minimal model in Eq. 11 to alternative models of varying complexity. To assess the predictive power of individual components, we performed model decompositions, where only a subset of components was used. We also examined more complex models which include other phenotypes of p53.

Models without selection. These models account for the mutation rates only and assume no selection on the mutations (fₘ=0). We consider a uniform model and a model of dinucleotide-specific frequencies. The predicted frequencies of mutations will therefore reflect the background distributions:

$${\hat{x}}_{m} = 1 / N , \qquad ( 20 )$$

$${\hat{x}}_{m} = {p}_{m} . \qquad ( 21 )$$

Partial models. In these models the background distribution of mutations is assumed to follow the dinucleotide-based estimation. We consider decompositions of the minimal fitness model into individual components:

$${f}_{m} = {σ}_{T} {T}_{m} , \qquad ( 22 )$$

$${f}_{m} ( H ) = {σ}_{I} {I}_{m} ( H ) . \qquad ( 23 )$$

Extended functional models. For extended models, we included all target genes in set G (WAF1, MDM2, BAX, h1433s, AIP1, GADD45, NOXA, and P53R2) for evaluating the transactivation component of the fitness model:

$${f}_{m} = {\sum }_{g ∈ G} {σ}_{T}^{g} {{T}^{g}}_{m} , \qquad ( 24 )$$

$${f}_{m} ( H ) = {\sum }_{g ∈ G} {σ}_{T}^{g} {{T}^{g}}_{m} + {σ}_{I} {I}_{m} ( H ) . \qquad ( 25 )$$

For each additional model, we retrained the parameters using TCGA-derived mutations and haplotypes.

Protein conservation and stability: We also evaluated an additional conservation/stability term for each p53 mutant. Conserved amino acids may play a role in function and protein structure, as well as protein-protein binding. Mutations in conserved amino acids may contribute to the cancer phenotype more so than not-conserved amino acids.

In brief, we infer conservation via evolutionary rates for each amino acid across commonly mutated tumor suppressors and oncogenes in cancer similar to the default parameters of the ConSurf server^{69,70}. For each protein sequence (except p53), we find homologous sequences available within the Uniref90 database^{62,71} using the phmmer software (HMMER Version 3.3.2, hmmer.org) with an E-value cutoff of 0.0001. We then select 150 equally-spaced homologous Uniref90 protein sequences. Next, we cluster the sequences using cdhit^{72,73} with a sequence identity threshold of 0.95. We then align the 150 homologous sequences with the original protein sequence using the MAFFT software (Version 7.475⁷⁴). Finally, we run the rate4site software (Version 3.0.0⁷⁵) to assign a standardized evolutionary rate to each amino acid of the protein sequence. For p53, we used 33 pre-determined homologous protein sequences across species used in previous work² using the ConSurf server with default parameters. The evolutionary rates are defined so that the average of them is zero and the standard deviation is one. Lower scores indicate increased conservation (lower evolutionary rate).

Previous work has suggested different mutants have different temperature-dependent stabilities that do not necessarily reflect functional binding of target DNA⁷⁶. The stability of the protein is quantified as the change in free energy between the natural and denatured state. If the change in free energy of folding is zero, then at equilibrium there will be an equal amount of folded and unfolded protein, as both the unfolded and folded states are equally likely. The fraction of stable folded protein, which is equivalent to the probability that the protein is folded properly (defined as Sₘ) is defined as:

$${S}_{m} = \frac{1}{1 + {exp}^{- Δ G / RT}} , \qquad ( 26 )$$

where R is the gas constant, T is the temperature in Kelvin, and ΔG is the change in free energy.

Previous work experimentally quantified ΔG for a number of mutants⁷⁶. Structural algorithms have been developed that are highly correlated to the experimental values previously reported⁷⁷. In order to quantify the changes in free energy for all possible mutants, we use values from the PBSA algorithm, as it had the highest linear correlation to experimental values⁷⁷. The PBSA algorithm only reports on the DNA-binding domain of p53 (here defined as amino acids between 96 to 289) as available crystal structures for p53 largely report on this region. For the other mutants outside of the defined DNA-binding domain, we assume they do not alter protein stability, and are assigned ΔG=0, which is consistent with the fact that these regions are largely disordered and difficult to structurally characterize⁷⁸.

The PBSA algorithm reports ΔΔG, which is defined as⁷⁷:

$$ΔΔ G = Δ {G}_{N - 0}^{M} - Δ {G}_{N - 0}^{W} , \qquad ( 27 )$$

where

$$Δ {G}_{N - D}^{M}$$

is the change in free energy for the mutant, and

$$Δ {G}_{N - D}^{W}$$

is the change in free energy for the wild-type, both for the natured-to-denatured direction. The value of

$$Δ {G}_{N - D}^{W}$$

has been reported by extrapolation to be approximately −3 kcal/mol⁷⁶. We then solve for

$$Δ {G}_{N - D}^{M}$$

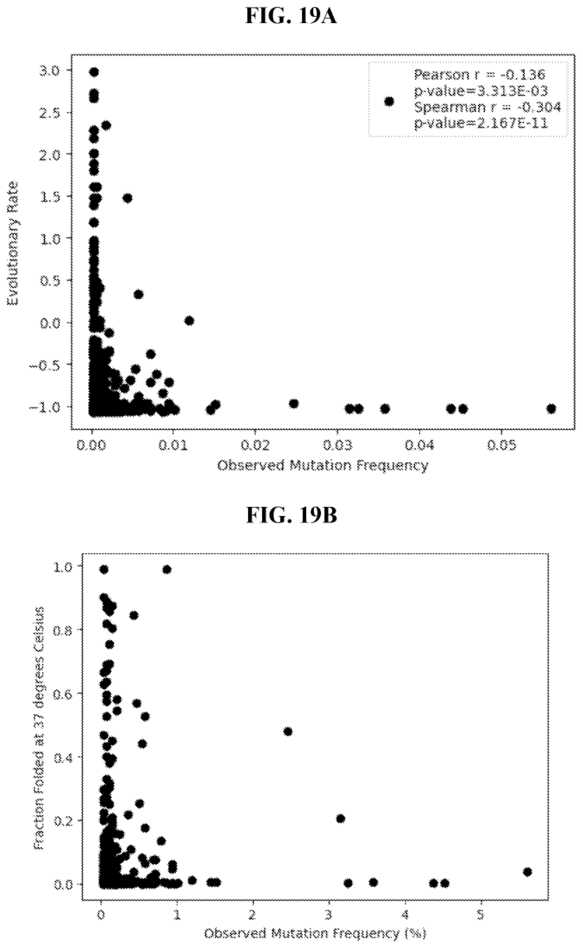

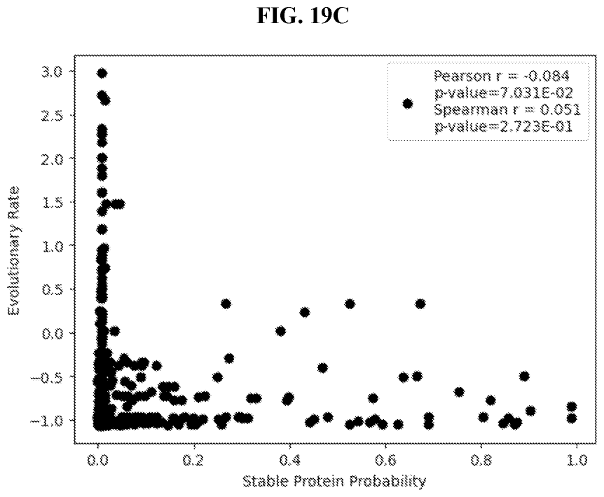

for the mutant change in free energy across mutants. Relationships between conservation, protein stability, and mutation frequency are presented in FIG. 19.

Models across commonly mutated tumor suppressors and oncogenes. For each considered gene, we compared the predicted conservation/function and immune phenotypes to each other via determination of rank correlations. Furthermore, we determined the degree to which hotspots were optimized for a certain phenotype, with respect to the other mutations within the same gene, using Welch's T-test. For all considered commonly mutated tumor suppressors and oncogenes, we also considered models of varying complexity. We considered models with only a dinucleotide background, as in Eq. 21. Additionally, defining the conservation term as σ_{C}Cₘ, we also considered models with only conservation predictions over a dinucleotide background,

$${f}_{m} = {σ}_{C} {C}_{m} , \qquad ( 28 )$$

only in silico immunogenicity predictions (as in Eq. 14) over a dinucleotide background,

$${f}_{m} = {σ}_{I} \frac{1}{{{K}_{{eff}_{I}}}^{m}} , \qquad ( 29 )$$

and combined models with both conservation and immunogenicity components over a dinucleotide background,

$${f}_{m} = {σ}_{C} {C}_{m} + {σ}_{I} \frac{1}{{{K}_{{eff}_{I}}}^{m}} , \qquad ( 30 )$$

For KRAS, there is additional function information available for seven hotspot mutants (G12A/C/D/R/V, G13D, and Qc1L)²⁵. We included the additional functional information for just these mutants as an additional level of complexity for modeling KRAS mutations,

$${f}_{m} = {σ}_{C} {C}_{m} + {\sum }_{q ∈ Q} {σ}_{F}^{q} {{F}^{q}}_{m} + {σ}_{I} \frac{1}{{{K}_{{eff}_{I}}}^{m}} , \qquad ( 31 )$$

where the term

$${\sum }_{q ∈ Q} {σ}_{F}^{q} {{F}^{q}}_{m}$$

considers functional phenotypes F^{q} within the set of Q functional phenotypes, which are intrinsic and extrinsically-assisted GTPase activity, as well as downstream binding to RAF effector protein. Notably, of these functional phenotypes for KRAS only the downstream binding to RAF effector was predictive of the KRAS mutation frequencies.

The structural term σ_{S}Sₘ is only available for p53. For p53, we consider fitness models that are extended by the protein conservation and stability terms, across two versions of the functional component, namely:

$${f}_{m} ( H ) = {σ}_{T} {T}_{m} + {σ}_{I} {I}_{m} ( H ) + {σ}_{C} {C}_{m} , \qquad ( 32 )$$

$${f}_{m} ( H ) = {\sum }_{g ∈ G} {σ}_{T}^{g} {{T}^{g}}_{m} + {σ}_{I} {I}_{m} ( H ) + {σ}_{C} {C}_{m} , \qquad ( 33 )$$

for conservation and

$${f}_{m} ( H ) = {σ}_{T} {T}_{m} + {σ}_{I} {I}_{m} ( H ) + {σ}_{S} {S}_{m} , \qquad ( 34 )$$

$${f}_{m} ( H ) = {\sum }_{g ∈ G} {σ}_{T}^{g} {{T}^{g}}_{m} + {σ}_{I} {I}_{m} ( H ) + {σ}_{S} {S}_{m} , \qquad ( 35 )$$

for stability.

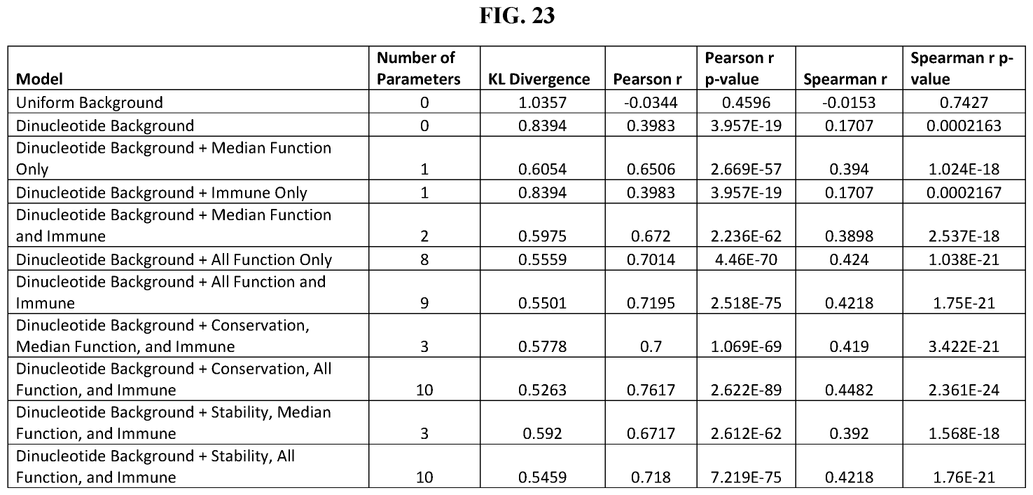

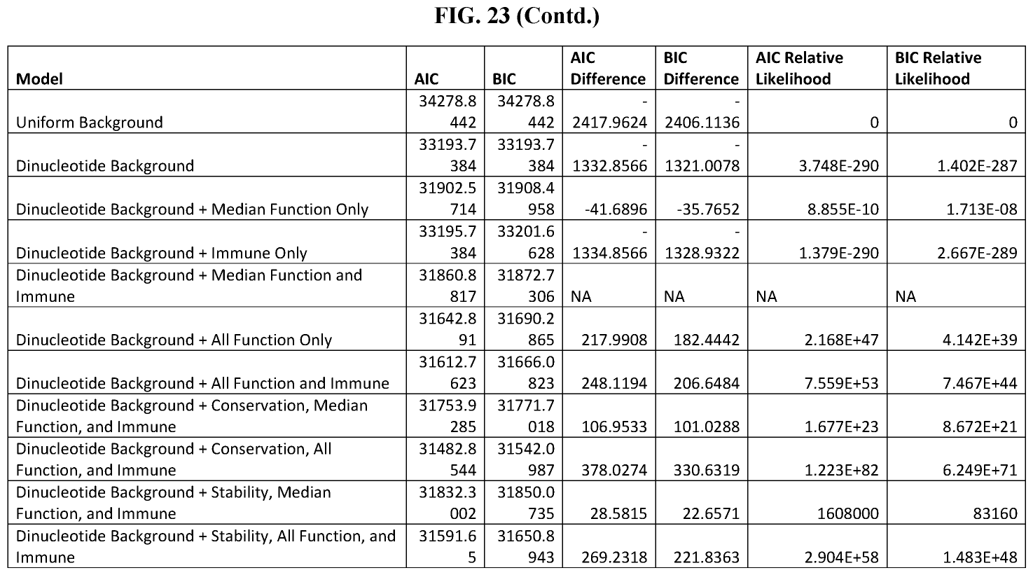

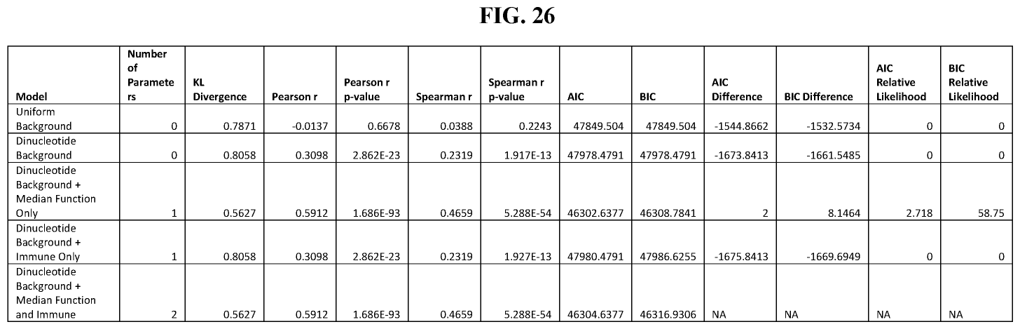

Predictive performance of models. For each model, we train parameters by maximizing data likelihood (Eq. 19), and compare the performance of the models in predicting the observed mutation frequencies in tumors (FIG. 23) as well as non-tumor mutated cells for p53 (FIG. 26). To compare between the models M of different complexity, which corresponds to the number of parameters, we utilize both the Bayesian Information Criterion (BIC) and the Aikake Information Criterion (AIC):

$$AIC = 2 ( k - ln ( \hat{L} ( {D}_{m} \vert {Θ}_{M}^{*} ) ) ) , BIC = k ln ( n ) - 2 ln ( \hat{L} ( {D}_{m} \vert {Θ}_{M}^{*} ) \qquad ( 36 )$$

where k is the number of parameters, n is the number of data points being fit, and

$${Θ}_{M}^{*}$$

is the set of parameters that maximizes the likelihood for model M (Eq. 19). BIC has a higher penalty for the number of parameters in a model for our case where there are many mutation frequency data points being fit. Each version of the fitness model is assigned an AIC and a BIC value, which depends on the number of parameters, the number of datapoints being fit (for BIC), and how well the data is fit. Model selection can be further justified by calculating the relative likelihood of models with respect to a reference criterion value corresponding to a reference model. We justify model selection by calculating the relative likelihood of models with respect to the two-parameter reference model (Eq. 11), which can be expressed as:

$${r}_{M} = exp [ ( {C}_{REF} - {C}_{M} ) / 2 ] , \qquad ( 37 )$$

where r_{M} is the relative likelihood value corresponding to model M, C_{M} is the criterion value corresponding to model M, and C_{REF} is the criterion value corresponding to the reference model. The criterion value can either be from AIC or BIC. The relative likelihood value quantifies how likely model M minimizes information loss with respect to the reference model. We evaluate r_{M} for all alternative models with respect to our two-component minimal fitness model, (Eq. 11), which is used as the reference model throughout the manuscript.

We additionally compute the rank (Spearman) and linear (Pearson) correlation coefficients and p-values for the observed and predicted frequencies, x and \hat{x}, respectively. The p-values are computed assuming a null distribution of correlation values derived from two independent t-distributions using the exact Pearson and Spearman probability density functions in the Python stats.pearsonr and stats.spearmanr functions from the scipy package.

Datasets. We have evaluated the performance of all models for mutant p53 with parameters fit on TCGA haplotypes and mutations on simulated haplotypes based on marginal NMDP HLA-I frequencies.

In the models we developed across tumor suppressors and oncogenes, which did not have concentration information, we evaluated the performance of all possible models with parameters fit on TCGA haplotypes and separately on TCGA and COSMIC mutation distributions for each gene. We fit models on both TCGA and COSMIC mutation distributions since the mutation distributions for many genes were less consistent between the databases as compared to p53.

Predictive performance: For p53, the two component minimal fitness model (Eq. 11) leads to predicted mutation frequencies that are strongly correlated to the observed mutation distribution in tumors. Moreover, consistently, as evaluated across the measures and reported in FIG. 23, models including both the functional and immune fitness components over-perform partial models, leading to predicted mutation frequencies that are strongly correlated to the observed mutation distribution. The addition of the immunogenicity component reduces the KL divergence of the predicted mutation frequencies with respect to the observed mutation frequencies and, despite increased model complexity, significantly improves model performance. Evaluation of the relative likelihood ratio (Eq. 37) demonstrates that the partial models have virtually no probability of minimizing information loss with respect to the minimal two-parameter model (Eq. 11). Moreover, while the two-parameter minimal model is highly predictive, we observe further increased predictive power of the extended models. These results illustrate how the proposed fitness model framework can be extended and can be used to gauge the importance of various phenotypes.

In predicting the non-neoplastic mutation distribution, addition of the immune component improved predictions to a lower degree than for the neoplastic mutation distribution. In the neoplastic setting, a combined model is ≃10⁷ times more likely to be a more appropriate model, whereas in the non-neoplastic setting the benefit was zero for a comparable sample size (2,764 mutation occurrences in TCGA versus 3,451 mutation occurrences in non-neoplastic settings) as determined via BIC (FIG. 26). This suggested that the role of the immune system in non-neoplastic cells may be smaller, which possibly depends on other genetic mutations, its environment, and how close the lesion is to becoming a neoplastic tumor.

For the models we tested across all examined cancer driver genes, we determined the appropriate model complexity via the Bayesian Information Criteria for both TCGA- and COSMIC-fit mutation frequency fit-ness models. We find that the appropriate model differs across the examined tumor suppressors and oncogenes, and that there is a positive relationship between the complexity of the model and the variance in the mutation frequencies for a particular gene. We find that genes with increased variance in mutation frequency are best explained by immunogenicity-only and combined models. These results illustrate the unique driving forces behind the mutation frequencies across diverse tumor suppressors and oncogenes, and show how minimal models successfully predict the mutation frequencies across these commonly mutated genes central in cancer development.

Effect of number of simulated haplotypes. We train p53 models on germline TCGA HLA haplotypes and TCGA mutations. TCGA haplotypes are directly inferred from TCGA samples and are not simulated. To investigate the effect of the number of haplotypes on the modeling results, we applied the same model weights to models with populations in the half-open interval [1, 10,000) simulated haplotypes in 100 haplotype steps, and in each case quantified the Kullback-Leibler divergence.

Internal validation. In the fitness model, each p53 mutation is assigned an effective background mutation rate, functional phenotype, and immune phenotype, where the phenotypes are linked by mutant p53 concentration. We investigated the consequences of shuffling these components on the model fitting. We posited that the fitness model was only appropriate based on the available experimental and computational data for TP53 mutations, and randomly shuffling these values should render phenotype data which the fitness model cannot appropriately fit. For each internal validation step, we randomly permuted the background mutational frequencies, functional phenotypes, and immune phenotypes 1,000 times and attempted each time to fit the reference two-parameter model (Eq. 11). In each iteration, the minimized Kullback-Leibler divergence is always an order of magnitude larger than the results with non-shuffled data, and we found that in no case were we able to fit a model as well as with the non-permuted data. This suggests that the fitness model presented would not be appropriate for randomly-generated datasets.

Relative immune weight. To quantify the relative contribution of the fitness components, we refactor our fitness expression to a form that is equivalent for predicting mutation frequency. To do so, we standardize the Tₘ and Iₘ distributions across mutations and haplotypes to an equivalent relative fitness form:

$$\begin{matrix}{\tilde{f}}_{m} ( \{ {σ}_{T} , {σ}_{I} \} , H ) = {σ}_{T} {s}_{T} ( \frac{{T}_{m} - {μ}_{T}}{{s}_{T}} ) + {σ}_{I} {s}_{I} ( \frac{{I}_{m} - {μ}_{I}}{{s}_{i}} ) \\ = {σ}_{T} {s}_{T} ( \frac{{T}_{m}}{{s}_{T}} ) + {σ}_{I} {s}_{I} ( \frac{{I}_{m}}{{s}_{I}} ) - [ \frac{{σ}_{T} {μ}_{T}}{{s}_{T}} + \frac{{σ}_{T} {μ}_{T}}{{s}_{T}} ] \\ = {( {σ}_{T} )}^{′} ( \frac{{T}_{m}}{{s}_{T}} ) + {( {σ}_{I} )}^{′} ( \frac{{I}_{m}}{{s}_{I}} ) - [ \frac{{σ}_{T} {μ}_{T}}{{s}_{T}} + \frac{{σ}_{I} {μ}_{I}}{{s}_{I}} ] ,\end{matrix} \qquad ( 38 )$$

where μ_{T,I} and s_{T,I} are the means and standard deviations of the Tₘ and Iₘ distributions, respectively, across mutations and haplotypes, and (σₜ)′=s_{T}σ_{T} and (σ_{I})′=s_{I}σ_{I}. Note that the fitness is translationally invariant, so the final constant term is not relevant for predicting the mutant frequencies. Therefore, the fitness as expressed in Eq. 38 is equivalent to the original fitness expression (Eq. 11) for predicting mutation frequencies, as the only difference between them is a constant.

The sum of the two rescaled fitness weights correspond to a particular amplitude A′. Note that both fitness expressions from Eq. 11 and Eq. 38 have only one degree of freedom despite the fact that there are two parameters, since (σ_{T})′=A′−(σ_{I})′. Therefore, Eq. 38 can be written as a linear function of (σ_{I})′ only. Knowing this, we can define the relative immune weight v_{I} as:

$${v}_{I} = \frac{\vert {( {σ}_{I} )}^{′} \vert}{\vert {( {σ}_{T}^{*} )}^{′} \vert + \vert {( {σ}_{I}^{*} )}^{′} \vert} , \qquad ( 39 )$$

where

$${( {σ}_{T}^{*} )}^{′} and {( {σ}_{I}^{*} )}^{′}$$

are optimized standardized weights. We derive s_{T}=0.178 and s_{I}=0.0106 from the full TCGA mutant TP53 Tₘ and Iₘ(H) distributions, respectively, across mutations, haplotypes, and tissues, as this is the data with which we train our fitness model.

To determine the optimal model for a cohort, we vary the parameter v_{I} over the interval [0,1] to determine the relative importance of the immune component to an optimized model. We do so by recomputing the logrank scores for Kaplan-Meier curves separated on the median mutant pQ3 total fitness defined for each v_{I} value.

### Free Fitness and Pareto Optimality

Free fitness. The fitness model predicts mutation frequencies based on three terms for each mutation: background mutation frequency pₘ, functional fitness

$${f}_{m}^{T} = {σ}_{T} {T}_{m} ,$$

and immune fitness

$${f}_{m}^{I} ( H ) = {σ}_{I} {I}_{m} ( H ) .$$

Moving the background mutation frequency into the exponent gives us an equivalent expression for predicting the mutation frequency:

$$\begin{matrix}{\hat{x}}_{m} ( H ) = {Z}_{H}^{- 1} {p}_{m} exp [ {f}_{m} ( H ) ] \\ = {Z}_{H}^{- 1} {p}_{m} exp [ {f}_{m}^{T} + {f}_{m}^{I} ( H ) ] \\ = {Z}_{H}^{- 1} exp [ ( log {p}_{m} + {f}_{m}^{T} ) + {f}_{m}^{I} ( H ) ] \\ = {Z}_{H}^{- 1} exp [ {F}_{m} ( H ) ] ,\end{matrix}$$

where we define the free fitness

$${F}_{m} ( H ) ≡ log {p}_{m} + {f}_{m}^{T} + {f}_{m}^{I} ( H ) ,$$

in an analogy to the free energy from statistical physics. We average across a population of MHC-I haplotypes to obtain

$${f}_{m}^{I} = {( {f}_{m}^{I} ( H ) )}_{H} .$$

To illustrate the free fitness landscape, we plot the coordinates of each mutation in a phenotypic space, denoted as

$${P}_{m} = ( log {p}_{m} + {f}_{m}^{T} , {f}_{m}^{i} ) .$$

We therefore plot the phenotypic consequence of all TP53 missense mutations derived from single nucleotide variants using the free fitness.

Pareto optimality. We compute the Pareto front for our data as follows: we query each mutation m and its corresponding point Pₘ in phenotype space and compare it to every other mutation n and its corresponding phenotype point Pₙ. A mutation not on the Pareto front is one for which there exists a point in phenotype space for which one feature is improved while the others are at least equal.

Specifically, for each pair of mutations m and n we consider the two differences between their coordinates in the phenotypic space:

$$( log {p}_{{m}_{j}} + {f}_{{m}_{j}}^{T} ) - ( log {p}_{{m}_{i}} + {f}_{{m}_{i}}^{T} ) = log ( \frac{{p}_{{m}_{j}}}{{p}_{{m}_{i}}} ) + ( {f}_{{m}_{j}}^{T} - {f}_{{m}_{i}}^{T} ) = {d}_{1} , \qquad ( 41 )$$

$$and$$

$${f}_{{m}_{j}}^{I} - {f}_{{m}_{i}}^{I} = {d}_{2} . \qquad ( 42 )$$

For a mutation mᵢ, if d₁ or d₂ are greater or equal to ϵ=0.1 for all other mutations in the phenotypic space, then point mᵢ is on the Pareto front. To illustrate the Pareto front, we draw a convex hull containing the Pareto front coordinate set using the shapely Python package smoothed using the following parameters: pareto_front.buffer(10, join_style=1, mitre_limit=50). buffer(−10, join_style=1, mitre_limit=50).

We then truncate the the convex hull based on the maximum of the intrinsic fitness,

$$log {p}_{{m}_{i}} + {f}_{{m}_{i}}^{T} ,$$

and the maximum of the immune fitness,

$${f}_{{m}_{i}}^{I} ,$$

and do not close the convex hull, allowing the Pareto front to be delimited by the Pareto optimal coordinate set. To obtain the optimal solution on the Pareto front, we calculate the point on the Pareto front with the maximum free fitness by discretizing the Pareto front into 10,000 equally spaced points and calculating the free fitness value for each point.

### Modeling Trade-Offs for Other Driver Genes

Utilizing the detailed functional information available for a number of KRAS hotspot mutations²⁵, we inferred the oncogenicity and the immunogenicity of these KRAS hotspot mutations in TCGA PAAD samples. Importantly, the only functional component which was predictive for fitting the KRAS mutational distribution was the downstream RAF protein effector binding. Therefore, for the functional “oncogenic” component, we determined the probability of a particular mutant KRAS binding downstream RAF effector protein in the MAPK pathway in a non-cooperative fashion, normalized by the number of KRAS alleles and assuming equal number of wild-type and mutant KRAS alleles as well as fully-active mutant KRAS. This component summarizes the likelihood of active, mutant KRAS binding RAF protein and transducing cell growth signaling:

$${P}_{RAF} = \frac{{L}_{KRAS}}{{L}_{KRAS} + {K}_{RAF}} , \qquad ( 43 )$$

where L_{KRAS} is the inferred concentration of mutant KRAS in a particular cancer cell, and K_{RAF} is the provided dissociation constant for KRAS-RAF protein binding from Ref. 38. For the immune component, we inferred the effective probability of mutant KRAS nonamer peptides being presented on matched HLA-I molecules, in a manner similar to Eq. 4.

There is no RPPA proteomic data available for KRAS in TCGA. In order to address this, we inferred the concentrations of KRAS in TCGA PAAD samples using KRAS RNA expression, calibrated using known wild-type KRAS concentrations in a WT/WT KRAS SW48 cell line. We infer the wild-type KRAS concentrations from Ref. 83, using the parental wild-type cell line. We assume a cell diameter of 20 micrometers,⁷⁹ a typical KRAS ploidy of two which means suggest ≃10⁵ KRAS protein molecules per allele, and a spherical cell shape. In brief, we assumed that all RNA expression was strongly linearly correlated to protein expression. Next, since the SW48 cell line is derived from a colon cancer, we calibrated the RNA expression to an expected concentration value across wild-type KRAS TCGA COAD tumors. This was done in an analogous way as for p53, where we inferred concentration using wild-type p53 BRCA RPPA data calibrated using a breast cancer-derived cell line with known wild-type p53 concentration. From this, we obtain an expected concentration of KRAS for each TCGA PAAD tumor cell. We further normalize by the number of alleles, assuming equal numbers of wild-type and mutant KRAS alleles.

As the protein concentration goes into both the oncogenic and immunogenic terms, cancer cells which upregulate mutant KRAS, for the purpose of increased cell growth, do so at the cost of increasing the concentration of the mutant antigen, implying a trade-off between the oncogenic potential of a mutant and its immune selection in upregulated oncogenes.

Validating Trade-Offs with ATAC-Seq and RNA

We predict functional fitness based on the yeast functional assay (see Section Inference of apparent dimer dissociation constants). We estimate downstream functional capacities of mutant p53 on target gene RNA expression in a tumor using ATAC-seq (Assay for Transposase-Accessible Chromatin with high-throughput sequencing) and RNA-seq data in matched TCGA samples. Previous work performed ATAC-seq on 423 TCGA samples across 23 cancer types, predominantly breast cancer⁸⁰. We leverage ATAC-seq transcription factor footprinting, using three TP53 motifs (M3698_1.02, M1929_1.02, and M3699_1.02) for which transcription depth and flank are measured. Increased depth indicates higher transcription factor occupancy and increased flank indicates increased increased chromatic accessibility by other factors⁸¹.

The flanking accessibility (A_{F}) and footprint depth (D_{F}) are computed for each TP53 motif (M) as follows:

$${A}_{F}^{M} = {log}_{2} [ \frac{\text{Flank Height}}{Background} ] , \qquad ( 44 )$$

$$and$$

$${D}_{F}^{M} = {log}_{2} [ \frac{\text{Footprint Base}}{\text{Flank Height}} ] . \qquad ( 45 )$$

We compute a lack of DNA binding score (N_{F}^{M}) for p53 for each motif M as:

$${N}_{F}^{M} = {2}^{\frac{{D}_{F}^{M}}{{A}_{F}^{M}}} . \qquad ( 46 )$$

As this value increases, either the depth increases and/or the flank decreases, indicating a lack of binding compared to background. For each sample with available data, we identified samples with one mutant TP53 allele. We extract the depth, flank, and determine the combined lack of binding score

$${N}_{F}^{M}$$

for each motif, which we use as a proxy for the likelihood p53 is not binding its DNA target motif. For each mutation, we define the effective lack of DNA binding score as the harmonic mean of the lack of binding scores across the three motifs in order to control for large outlier values:

$${N}_{F} = \frac{3}{{\sum }_{M} {2}^{- \frac{{D}_{F}^{M}}{{A}_{F}^{M}}}} . \qquad ( 47 )$$

We consider RNA expression in Transcripts per Million (TPM) of the eight p53 target genes previously examined in the yeast assay (WAF1, MDM2, BAX, h1433s, AIP1, GADD45, NOXA, and P53R2). There are 373 TCGA samples with matched ATAC-seq and RNA-seq data.

Independently, we also consider the chromatin accessibility on regulatory regions for the eight target genes where such data is available⁸⁰. This was the case for only six target genes (WAF1, BAX, h1433s, AIP1, GADD45, and NOXA). Each gene may have multiple regulation sites, and each site has an associated number of Tn5 transposase insertion events which correlate to the site's chromatin accessibility. We transform the accessibility of these regulation sites into a probability distribution as follows. First, we define the chromatin accessibility of each gene G as G_{A}, which is the sum of the insertions across all regulatory sites:

$${G}_{A} = {\sum }_{r} {I}_{r} , \qquad ( 48 )$$

where r is a regulatory site and Iᵣ is the number of Tn5 transposase insertions corresponding to a regulatory site. This takes into account both the number of regulatory sites and the accessibility of these sites. For each sample, we define the median target gene accessibility S_{A} as the median number of gene insertions across all of the target genes' regulatory sites:

$${S}_{A} = {median}_{G} ( {G}_{A} ) . \qquad ( 49 )$$

Finally, we transform the distribution of S_{A} into a probability distribution via the softmax function, where P(A) is the probability the p53 target genes are accessible in a particular sample:

$$P ( A ) = \frac{exp {S}_{A}}{{\sum }_{S} exp {S}_{A}} \qquad ( 50 )$$

We then define the probability of p53 binding target DNA P(B) as follows:

$$P ( B ) = \frac{P ( B \vert A ) P ( A )}{P ( A \vert B )} . \qquad ( 51 )$$

where P(B|A) is the probability of p53 binding DNA given it is accessible, which is derived from the yeast assay and the mutant's typical concentration (see Sections Inference of apparent dimer dissociation constants, Inference of tissue- and mutant-specific concentrations, and Fitness Model), P(A) is the sample's target gene regulatory site accessibility probability, and P(A|B)=1, where if p53 is binding DNA then it follows that the DNA is by accessible. Therefore, the probability of p53 binding DNA is conditioned on the probability of the target genes having sufficient chromatin accessibility.

### Patient Data

Immunotheraphy-treated non-small cell lung cancer cohort. Patients were those with metastatic non-small cell lung cancer (NSCLC) treated with PD-(L)1 blockade-based immunotherapy between 2013-2019. Those treated with concurrent PD-(L)1+ cytotoxic chemotherapy were excluded. To be included, patients had to have molecular next-generation sequencing results by MSK-IMPACT as well as available outcomes data from their response to PD(L)1 therapy. Objective overall response and progression-free survival outcomes were determined by RECIST, performed by a blinded thoracic radiologist. Patients who did not progress were censored at the time of their last available imaging assessment. Overall survival was determined from the start of PD-(L)1 blockade until date of death; those who were still alive were censored at the time of last contact.

National Cancer Institute Li-Fraumeni Syndrome cohort. A total of 82 individuals carrying either pathogenic or likely pathogenic missense germline TP53 variants from the National Cancer Institute (NCI) LFS cohort (NCT014434c8; http://lfs.cancer.gov)⁸² were included. All participants or their legal guardians signed informed consent. As of Mar. 24, 2020, 52 carriers had developed at least one cancer while 30 had remained cancer-free. Non-melanoma skin cancers and HPV-associated high grade dysplasias were excluded from the cancer count. Genotyping was conducted using the Illumina Infinium Global Screening Array-24 (Illumina Inc. San Diego) at the Cancer Genomics Research Laboratory (CGR) in the Division of Cancer Epidemiology and Genetics (DCEG). HLA alleles were imputed with the tool HIBAG⁸³ using a model trained for European ancestry.

### Experimental Methods

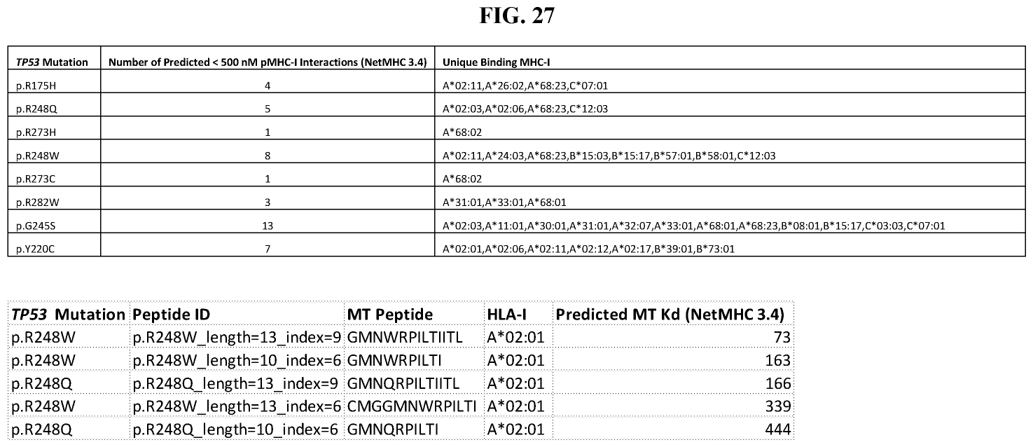

Peptide predictions. The HLA molecules predicted to present hotspot peptides using NetMHC 3.4^{61,63,64} are reported in FIG. 27. The HLA-A*02:01 allele is the most common HLA-I in TCGA. We aimed to infer differential potential immunogenicity between TP53 R175H and R248Q/W mutations, as these hotspots lie on different end of the trade-off between loss of function and potential neoantigen immunogenicity. We inferred all mutant peptides of 8-14 amino acids in length that cover the R175H and R248Q/W mutations and predicted IC50 affinities to HLA-A*02:01 using the NetMHC 3.4. All peptides with predicted affinities less than 500 nM are presented in FIG. 27. Only peptides corresponding to the R248Q/W mutations passed this filter, and we used the 10-mer peptides for the in vitro assays, as they were more likely to be presented. For R175H, we considered the HMTEVVRHC pep-tide (SEQ ID NO: 1), as previous work implied this peptide can be presented on HLA-A*02:01²⁸ although the predicted affinity was 10716 nM.

T2 binding assay. The TAP2-deficient human lymphoblastoid cell line T2 was maintained in RPMI-1640 supplemented with 7.5% FBS, NEAA, 2 mM L-glutamine and penicillin/streptomycin. Prior to assay setup, T2 cells were washed three times in serum-free RPMI-1640 and then plated at a concentration of 1×106/mL in serum-free RPMI-1640 with 5 μg/mL recombinant human (rh)β2 microglobulin (Sigma-Aldrich, cat. no. 475828) and 1, 10 or 100 μg/mL of peptide (×85% purity, Genscript) or DMSO as vehicle control and incubated overnight. The following day, cells were washed and stained with a fixable viability dye (Zombie NIR, 1:8000, BioLegend, cat. no. 423106) in PBS for 15 min on ice. Cells were then washed and stained with a FITC-conjugated anti-human HLA-A*02 antibody (clone BB7.2, 1:100, BD Biosciences, cat. no. 551285) for 30 min on ice in PBS. After staining, cells were washed and resuspended in PBS for acquisition on a 4-laser Aurora full spectrum cytometer (UV-V-B-R, Cytek). Data were analyzed using FlowJo software (version 10.7.1).

Human samples. All patients and healthy donors signed an approved informed consent before pro-viding tissue samples. Patient samples were collected on a tissue-collection protocol approved by the MSKCC Institutional Review Board. Peripheral blood mononuclear cells (PBMCs) from HLA-A*02:01 healthy donors and patients with TP53 R175H or R248Q mutant bladder or ovarian cancer were isolated from whole blood collected in CPT tubes containing sodium heparin (BD Vacutainer) according to the manufacturer's instructions. PBMCs from cancer patients were cryopreserved in FBS containing 10% DMSO until use. PBMCs from healthy donors were plated in 10 cm tissue culture dishes at 4-6×106 cells/mL in RPMI-1640, supplemented with 1% human serum (pooled male AB, Sigma-Aldrich, cat. no. H4522), 10 mM HEPES, 2 mM L-glutamine, and 50 μM 2-β-mercaptoethanol and incubated at 37 C for one hour. Non-adherent cells were washed off with PBS and cryopreserved in FBS containing 10% DMSO until further use. Adherent cells were cultured for 7 days in RPMI-1640 with 1% human serum, 1000 IU/mL rhGM-CSF, and 500 IU/mL rhIL-4 to induce differentiation of monocytes into monocyte-derived dendritic cells (mDCs). CD4+ and CD8+ T cells were isolated from the non-adherent cell fraction using human CD8 Microbeads (Miltenyi, cat. no. 130-045-201) and the human CD4+ T cell Isolation Kit (Miltenyi, cat. no. 130-0ϵ6-533) according to the manufacturer's instructions. CD4+ T cells were activated with 10 μg/mL PHA and cultured in the presence of 10 IU/mL rhIL-2 and 20 ng/mL rhIL-7 for one week before using them as CD4+ Th-APCs in peptide restimulation assays.

In vitro peptide stimulation assays. We used two types of in vitro peptide stimulation assays: one for inducing de novo priming of mutant p53 specific T-cell responses from healthy donors and one for recalling memory responses against mutant p53 peptides in T cells from patients bearing mutant p53 tumor lesions. To induce functional de novo priming of human CD8+ T-cells, we developed an optimized in vitro restimulation system (method manuscript in preparation). Briefly, CD8+ T-cells from HLA-A′Y02:01 healthy donors were stimulated with autologous mDCs pulsed with 10 μg/mL p53 peptides (>85% purity, Genscript), CEF (CEF-Class I peptide pool, 1:20, CTL), 1 μg/mL 15-mer HIV GAG peptide pool (JPT), or DMSO at a 5:1 ratio in RPMI-1640 supplemented with 10% FBS, NEAA, 2 mM L-glutamine, penicillin/streptomycin, 1 mM sodium pyruvate, and 50 μM β-mercaptoethanol (complete media) in the presence of 100 IU/mL rhIL-2 and 10 ng/mL rhIL-15. After one week of culture, cells were washed, and re-stimulated with peptide-pulsed, PHA-activated autologous CD4+ Th-APCs at a 1:1 ratio. Cultures were maintained in 100 IU/mL rhIL-2 and 10 ng/mL rhIL-15 for a second week. Cells were then washed and incubated with the specific peptides before intracellular cytokine staining by flow cytometry. To recall mutant p53 T-cell responses, patients PBMCs were stimulated with 10 μg/mL R175H and/or R248Q p53 (>85% purity, Genscript), CEF (CEF-Class I peptide pool, 1:20, CTL) as positive control, or DMSO as negative vehicle control in complete media in the presence of 10 IU/mL rhIL-2 and 10 ng/mL rhIL-15. Cells were restimulated with the respective peptides on day 7, and cultures were maintained with rhIL-2 and rhIL-15 for a second week. On day 15, cells were washed, restimulated with the specific peptides before intracellular cytokine staining by flow cytometry.

Intracellular cytokine staining by flow cytometry. Monensin (1-2 μM, BD GolgiStop, BD Pharmingen) was added 1 hour after the last peptide restimulation to inhibit intracellular protein transport and cultures were incubated for additional 5 hours. Cells were then washed and stained with an eFluor 506 (1:1000, eBioscience, cat. no. 65-0866-18) or Zombie NIR (1:8000, BioLegend, cat. no. 423106) fixable viability dye in PBS for 15 minutes on ice, followed by a 15-minute incubation with human Fc blocking reagent (1:10, Miltenyi) in 2% FBS PBS on ice, before staining with the following fluorochrome-conjugated surface antibodies: anti-human CD3-BUV395 (1:100, BD Biosciences, cat. no. 740283), anti-human CD4-BV650 (1:50, BD Biosciences, cat. no. 563875) or CD4-AlexaFluor700 (1:50, Invitrogen, cat. no. 56-0047-42), and anti-human CD8-BUV563 (1:50, BD Biosciences, cat. no. 612914) or CD8-AlexaFluor647 (1:50, BD Biosciences, cat. no. 557708), anti-human CD45RA-BUV737 (1:100, BD Biosciences, cat. no. 564442), and anti-human CD62L-PE (1:100, BD Biosciences, cat. no. 555544). After 40-minute incubation on ice, cells were washed and subsequently fixed and permeabilized using the FoxP3/Transcription Factor Staining Buffer Set (Thermo Fisher Scientific, cat. no. 00-5523-00). Intracellular staining was performed in permeabilization buffer for 45 minutes on ice with the following anti-bodies: anti-human IFN-γ-FITC (1:50, Invitrogen, cat. no. BMS107FI), anti-human TNF-α-PE-Cy7 (1:50, BD Biosciences, cat. no. 557647) and anti-human Ki67-APC-eFluor 780 (1:1600, Invitrogen, cat. no. 47-5698-82). Cells were washed in permeabilization buffer and resuspended in PBS for acquisition on a 4 laser Aurora full spectrum cytometer (UV-V-B-R, Cytek). Data were analyzed using FlowJo software (version 10.7.1).

Multiplex Identification of Antigen-Specific T Cell Receptors (MIRA) assay. To compare the relative immune fitness of TP53 mutations depending on the position of their amino acid substitutions, we used MIRA to search for TCRs against mutant p53 in naive CD8 T cell repertoires of healthy donors. MIRA com-bines conventional immunological techniques with high-throughput TCR sequencing to identify antigen specific T cells in high-throughput through the sorting and sequencing of T cells activated in response to pools of peptide epitopes⁸⁴.

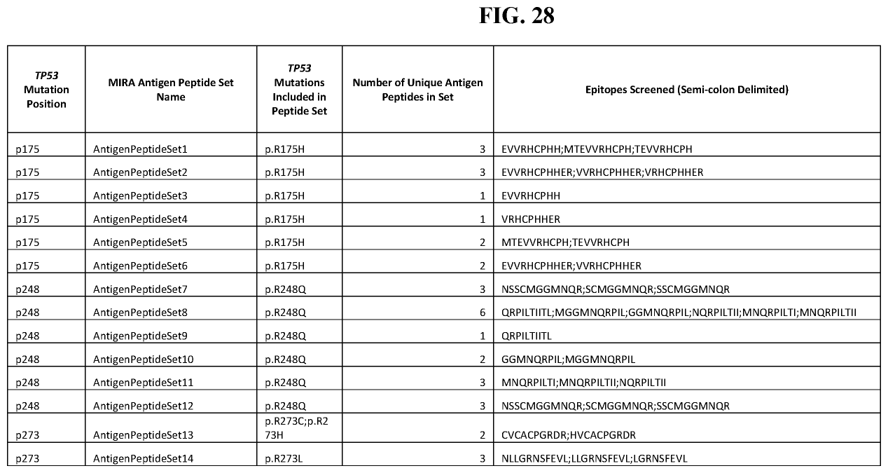

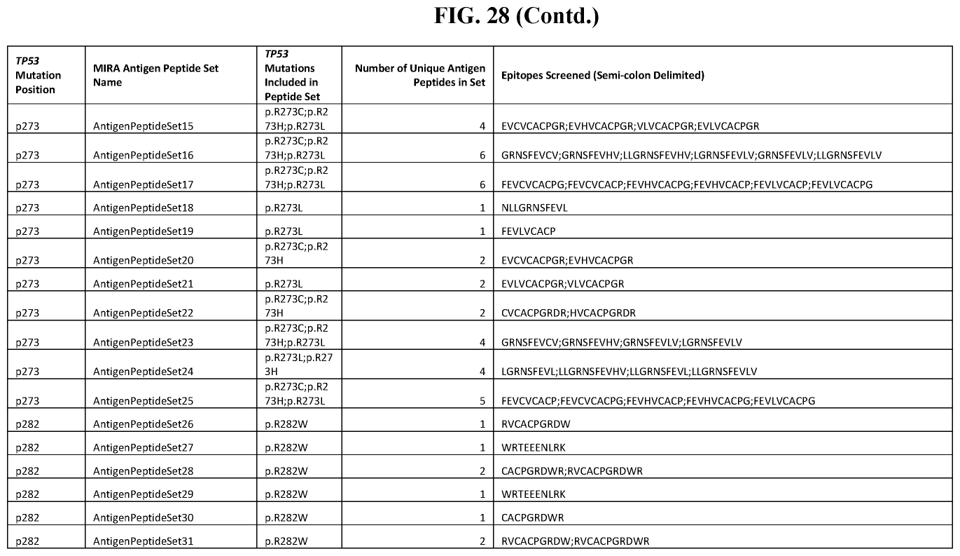

We synthesized 40 distinct 9-11 length peptide epitopes that encompassed common p53 mutations at positions pR175 (H), pR248 (Q), pR273 (C/H/L), and pR282 (W) and which were predicted to bind to at least one of 60 common HLA class I alleles by NetMHCpan version 4.1⁸⁵ (FIG. 28). Peptide synthesis was performed by GenScript (Piscataway, NJ). The 40 peptides were pooled in a combinatorial fashion as described previously⁸⁴, where peptides with high sequence similarity were grouped together into discrete antigen sets. Each antigen set was placed in a unique subset of 6 out of 11 peptide pools labelled A-K, hereafter referred to as the antigen's occupancy.

We acquired Leukopaks from 107 healthy donors from a variety of commercial sources (AllCells, Alameda CA & Bloodworks Northwest, Seattle WA). Donors represented diverse HLA Class I backgrounds, encompassing 25 distinct HLA-A alleles, 46 HLA-B alleles, and 20 HLA-C alleles at 4 digit typing. 100/107 donors had at least one A*02:01 allele. There were 103 unique MHC-I haplotypes. We conducted a total of 222 MIRA experiments on average 2 experiments per donor.

MIRA experiments were performed as follows: naive CD8 (nCD8) T cells were isolated from donor Leukopaks and 30-200 million nCD8s were co-cultured for 12-14 days with monocyte-derived dendritic cells pulsed with the entire set of query peptides in the presence of cytokines GM-CSF/IL-4/IFN-g and LPS. T cells were supplemented with IL-7 and IL-15 on day 3 of the expansion. Following a 12-14 day expansion, the T cell culture was split into replicate aliquots and T cells were re-stimulated with MIRA-formatted peptide pools at 37 C for 16 hours. Sorting was done on CD3+CD8+CD137+ T cells and followed similar preparation and sequencing of the TCRb locus as reported in Ref. 90. T-cell presence was assessed by aggregating the behaviour of specific TCRb sequences across sorted pools and we utilized a non-parametric Bayesian model described previously⁸⁶ to identify T-cell clonotypes with read count patterns consistent with enrichment in 6 of the 11 replicate antigen exposures (also described in Ref 88).

We considered all TCR-antigen associations with a posterior probability of >=0.5 to represent a significant response to the antigen at that occupancy, then counted the number of TCRs that responded to antigens with each of the p53 p175, p248, p273, and p282 mutations. To permit fair comparison of the number of TCRs yielded between each of the TP53 positional mutants, we calculated each donor's average count of TCRs yielded per antigen peptide by (1) the number of peptides in the MIRA antigen set (i.e. the number of putative epitopes at that occupancy), (2) the number of MIRA antigens (i.e. occupancies) representing each of the four TP53 positional mutants, and (3) each donor's number of experiments. This procedure yielded a single value representing each of the 107 donors' average number of TCRs yielded per antigen peptide, for each of the TP53 p175, p248, p273, and p282 MIRA antigen groups.

We reasoned that TP53 positional mutation antigen groups with lower immune fitness should yield higher normalized TCR yield from these 107 healthy donors. To test for significant differences in normalized TCR yield, we conducted a two-sided Mann-Whitney U Tests on normalized TCR yield values for each pairwise combination of p175, p248, p273, and p282.

Selection of representative cancer driver genes and hotspots. A total of 27 representative tumor suppressors and oncogenes implicated in driving tumorigenesis and commonly mutated in TCGA were selected^{1,87}. These genes are: KRAS, HRAS, NRAS, PTEN, PIK3CA, PIK3R1, EGFR, BRAF, NOTCH1, RB1, ARID1A, MYC, POLE, MLH1, MSH2, MSH3, IDH1, CDKN2A, CTNNB1, ERBB2, SMAD2, SMAD4, APC, BRCA1, BRCA2, FAT4, and TP53. Only missense mutations, which are amenable to our model predictions, were considered because for example, there are fewer doubts concerning mutant protein expression. Hotspots from TCGA were manually curated. The genes and their hotspots are shown below.

Selected cancer driver genes and hotspots

| Gene | Hotspots |
|---|---|
| KRAS | G12D, G12V, G12C |
| HRAS | Q61R |
| NRAS | Q61R, Q61K |
| PTEN | R130Q, R130G |
| APC | S2307L |
| PIK3CA | E545K, H1047R, E542K |
| PIK3R1 | G376R, N564D |
| EGFR | L858R, A289V, G598V |
| BRCA1 | E1258D |
| BRCA2 | A1393V, E3342K |
| BRAF | V600E |
| NOTCH1 | A465T |
| RB1 | R876C, R741C, R451C |
| ARID1A | G2087R |
| MYC | S161L |
| POLE | P286R, V411L |
| MLH1 | R265C, R385C |
| MSH2 | R929Q, R406Q, K871N |
| MSH3 | A841T, D479Y, K757N, I1082M, D143N |
| IDH1 | R132H, R132C |
| CDKN2A | H83Y, P114L, D108Y |
| CTNNB1 | S37F, T41A, S45P |
| ERBB2 | S310F |
| SMAD2 | R120Q, S276L, R321Q |
| SMAD4 | R361H, R361C |
| FAT4 | R2685Q, R1671C, D1790N, H2514Y |
| TP53 | R175H, R248Q, R273H, R248W, R273C, R282W, |
| | G245S, Y220C |

Selection of representative genes and mutations implicated in non-cancer diseases. We tested if mutations which are less conserved are more likely to generate more immunogenic peptides (as defined by likelihood to be presented on class I MHC), outside of the cancer setting. To do so, dozens of genes which have single nucleotide polymorphisms that are associated with non-cancerous diseases were examined. Any gene for which there was at least some evidence that it had functional importance for cancer development, or whose symptoms manifested as benign tumors were filtered out. We kept genes in which, to date, mutations only have strong documented evidence for roles in non-cancerous diseases.

A total of nine genes were considered. Five of these genes are hemoglobin subunits (HBA, HBB, HBD, HG1, HG2), and the other four are related to other non-cancer associated conditions (PAH, F8, PHEX, POGZ). Mutations in hemoglobin subunits are well-documented, mainly the HBA and HBB subunits which are the major hemoglobin subunits in adults^{88,89}. While some mutations are benign and do not alter hemoglobin function or stability, there are multiple mutations which are functionally destructive. Mutations in phenylalanine hydroxylase (PAH) are associated with phenylketonuria, resulting in reduced phenylalanine metabolism⁹⁰. Mutations in Factor VIII (F8) contribute to hemophilia A⁹¹. Mutations in phosphate-regulating neutral endopeptidase, X-linked (PHEX) are related to bone deformations due to inhibited phosphate retention⁹². Mutations in the pogo transposable element with ZNF domain (POGZ) gene are related to White-Sutton syndrome⁹³. In all cases, mutations within the genes in question may have a spectrum of functional effects, from negligible changes to significant alterations in function or protein stability.

Single-nucleotide polymorphism data for these genes available from the NCBI's dbSNP⁹⁴ were collated and genomic mutations mapped to amino acid alterations using the GRCh38 reference genome, identifying a total of 2,195 missense mutations across these 9 genes. We then only kept the mutation set which were unequivocally not-pathogenic (annotated as “benign”, “protective”, “likely-benign”, and/or “benign-likely-benign”) or pathogenic (annotated as “pathogenic”, “likely-pathogenic”, and/or “pathogenic-likely-pathogenic”) as determined by the NCBI's ClinVar annotation system⁹⁵. This resulted in 113 not-pathogenic mutations and 836 pathogenic mutations for a total of 949 mutations. All other mutations were not considered for the analysis.

For each gene, inferred population-averaged likelihood of class-I MHC presentation for the nine 9-mer peptides surrounding the mutation across the “non-pathogenic” (i.e., more sequence conservation) and “pathogenic” (i.e., poor sequence conservation) groups we compared as described herein.

Mutation datasets. Our models are applied to somatic mutations across commonly mutated tumor suppressors and oncogenes, as well as pre-neoplastic TP53 mutations. For mutant p53, we train the mutation model on somatic TCGA TP53 mutation distributions downloaded from the Genomic Data Commons.⁸⁷ We consider a total of 2,764 p53 mutations across 2,580 tumors in TCGA. We only consider missense mutations which arise from a single-nucleotide variation.

In examining models without concentration for all considered commonly mutated tumor suppressors and oncogenes, we utilized missense mutation distributions from both COSMIC (version 90)⁹⁶ and TCGA, as available from the Genomic Data Commons⁸⁷. We only considered missense mutations from single-nucleotide variations to limit confounding issues with protein expression in other types of mutants, such as truncation mutants. Where possible, we assured that we considered properly matched primary canonical transcripts of these genes across databases. For KRAS, where there are two well-expressed isoforms which have largely conserved amino acid sequences, we focused on isoform “A”, which is listed as the canonical transcript in the UniProt database⁹⁷.

It has become clear in recent years that TP53 mutations exist in cells which are non-cancerous³⁰. To date, there is no large-scale non-tumor somatic p53 mutation database which collates data from multiple sources, such as IARC does for p53 mutations in tumors and in patients with Li-Fraumeni Syndrome. To address this, we assembled SNV-generated missense TP53 mutations in non-tumor tissues across 17 publications into one non-neoplastic TP53 mutation database, collating 3,541 missense mutation occurrences (3,135 of which are in the DNA binding domain, defined here as amino acids [100, 300]), comparable in order-of-magnitude to other databases such as IARC R20 Europe (N=7,579) and TCGA (N=2,764). We gathered mutations in the blood from eight datasets⁹⁸⁻¹⁰³, urothelium mutations in one dataset¹⁰⁴, bladder mutations in one dataset¹⁰⁵, bronchial mutations in three datasets¹⁰⁶⁻¹⁰⁸, colorectal mutations in three datasets¹⁰⁹⁻¹¹¹, gynecological mutations in seven datasets¹¹²⁻¹¹⁸, esophageal mutations in nine datasets¹¹⁹⁻¹²⁷, liver mutations in one dataset¹²⁸, skin mutations in ten datasets¹²⁹⁻¹³⁷, and four pan-tissue datasets¹³⁸⁻¹⁴¹. In all cases we assured that only mutations which were identified as not being cancer-derived were included.

For the Li-Fraumeni IARC mutation distribution, we used the R20 version of the IARC germline database⁴⁸. We excluded all data which may have been contributed by the NCI, in order to avoid analyzing survival for the same person twice. We only considered missense mutations.

Kaplan-Meier Curves. We examined the role of inferred mutant p53 functional, immune, and total fitness on survival in both non-immunotherapy treated (TCGA, pan-cancer) and immune checkpoint-blockade (ICB)-treated (non-small cell lung cancer, Memorial Sloan Kettering (MSK)) cohorts. For the IARC R20 Li-Fraumeni patients with germline TP53 mutations, we plotted a Kaplan-Meier curve for first age of onset of a tumor. In all cases we estimated the mutant fitness using the inferred tissue-specific concentration and the matched haplotype where possible. We used the matched mutant and haplotype for defining the immune fitness for all cohorts except for the IARC R20 Li-Fraumeni cohort. For the IARC Li-Fraumeni cohort, we infer the haplotype using TCGA haplotype distribution.

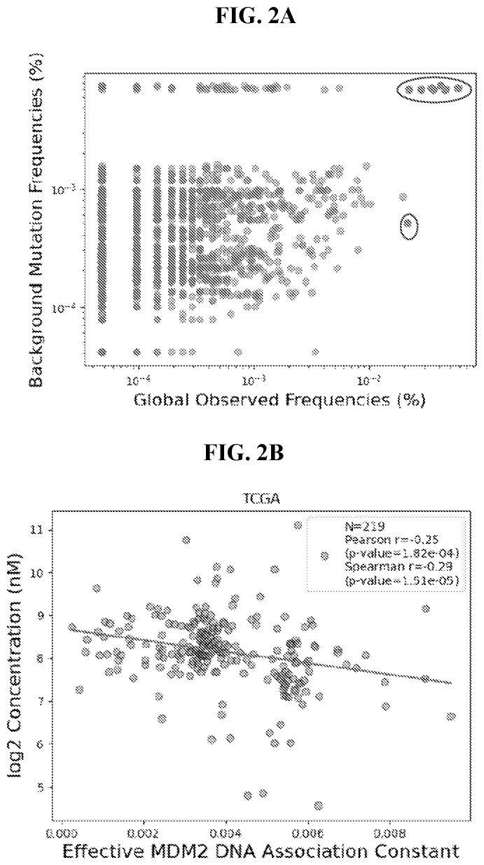

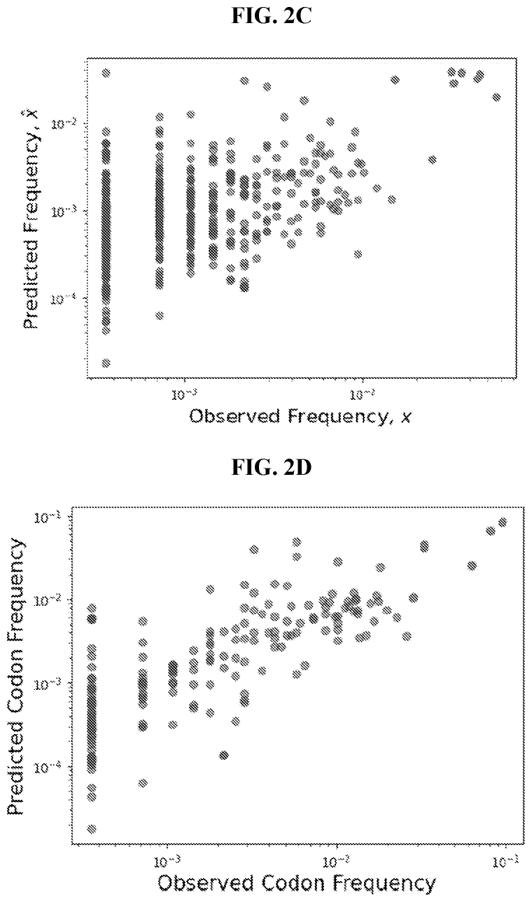

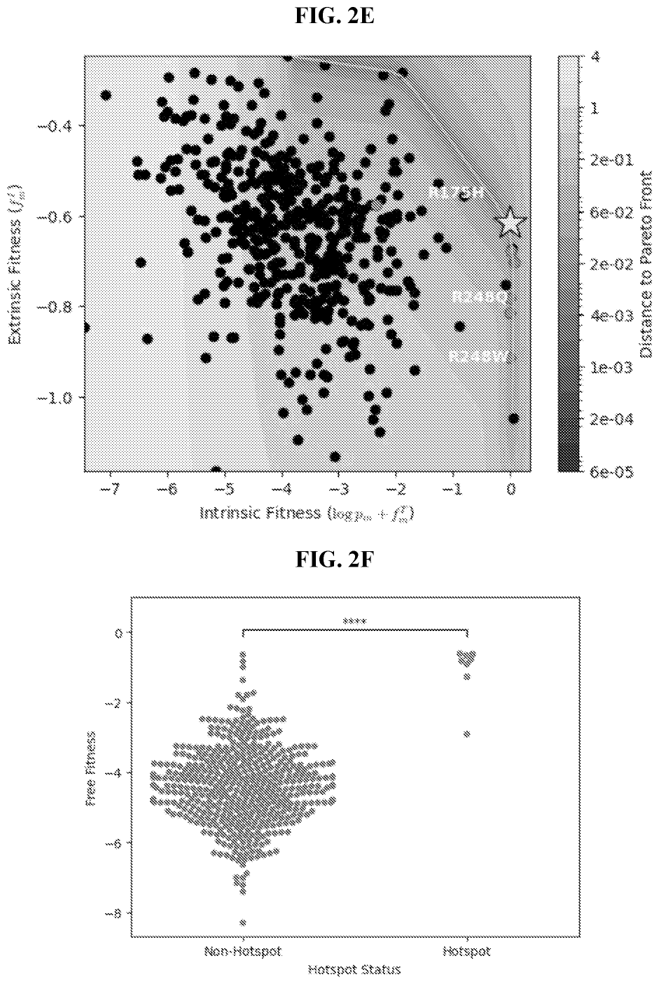

Description of statistical methods. We used Welch's T-test and the Mann-Whitney U-test for categorical tests. We used the Pearson and the Spearman correlations for continuous variables. For model training and testing, we calculated the Kullback-Leibler divergence using the observed and predicted mutation frequencies. The confidence intervals in SI FIG. 2 are 95% confidence intervals computed using the normal approximation. The log-rank test is used for testing separation significance in Kaplan-Meier curves.

### Example 2: Models for Predicting Fitness of P53 Mutations

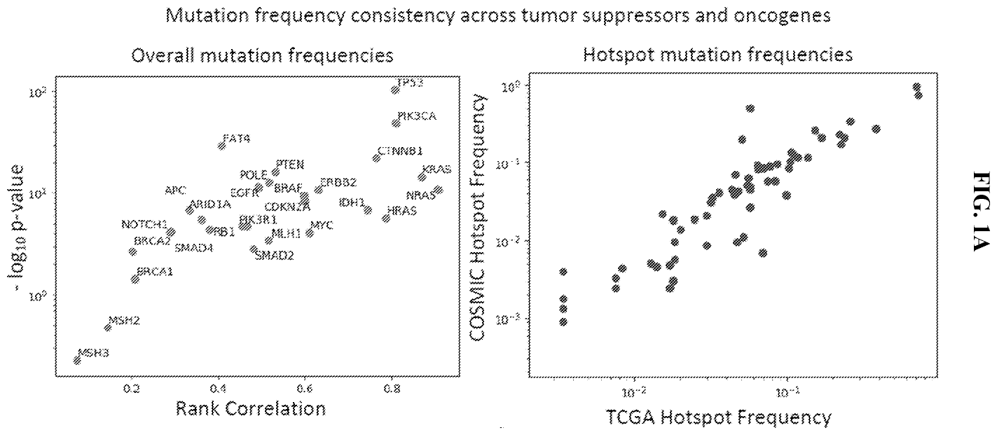

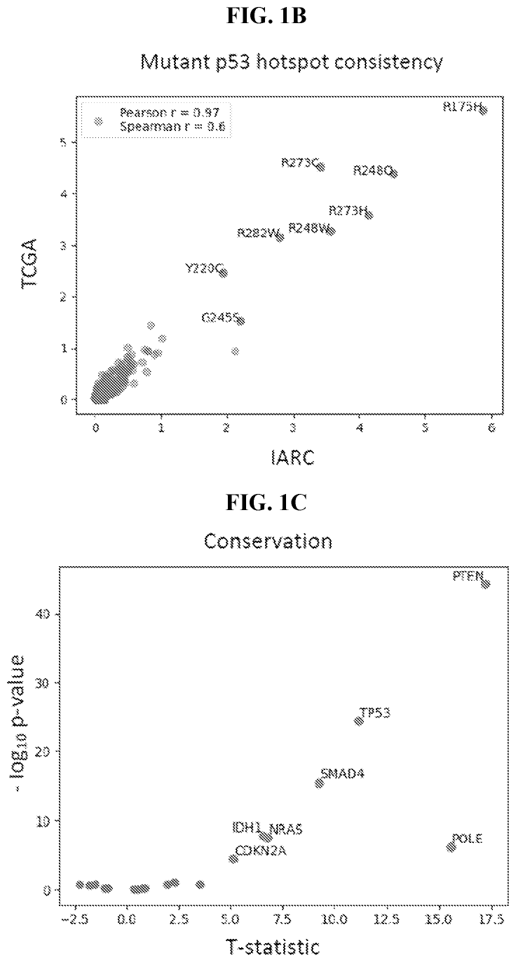

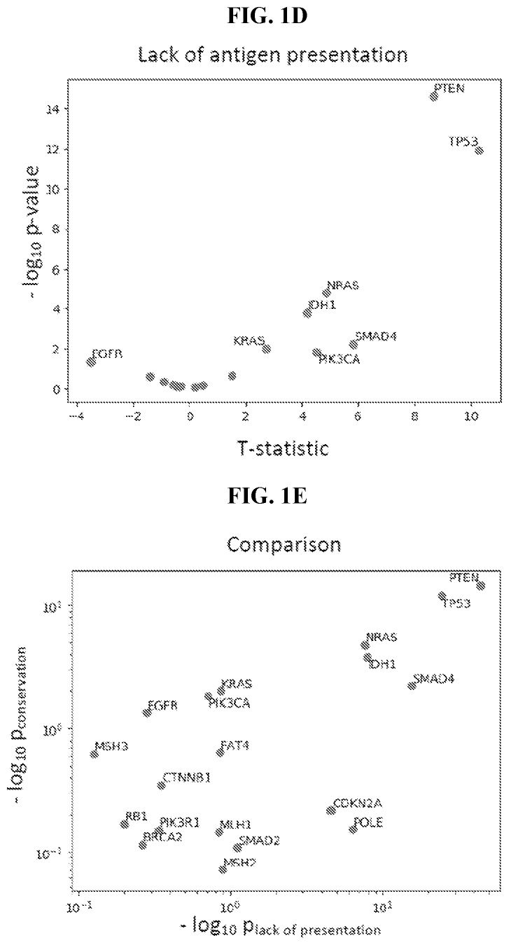

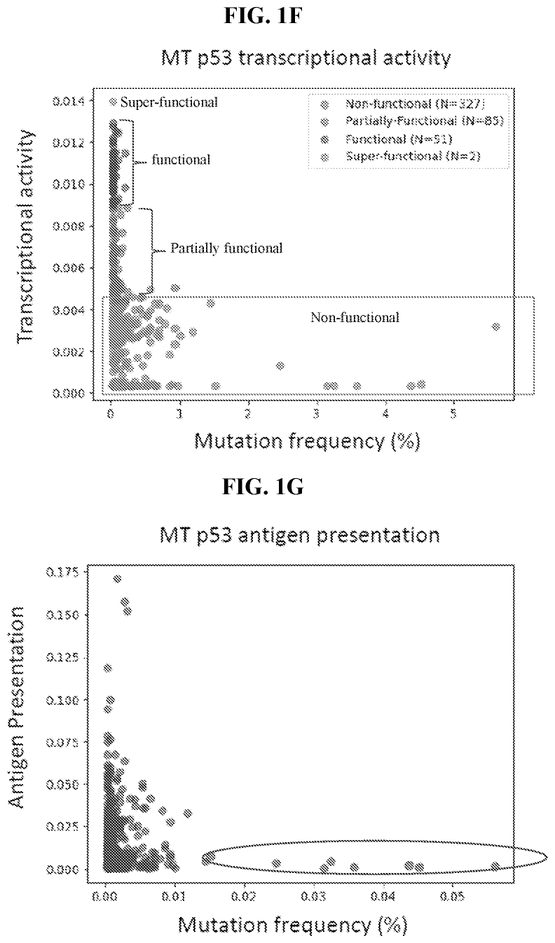

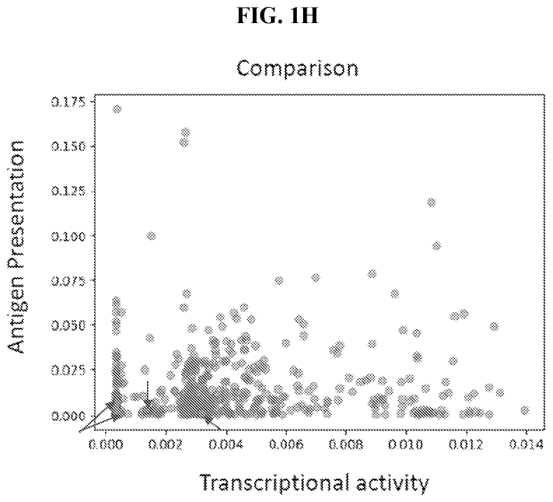

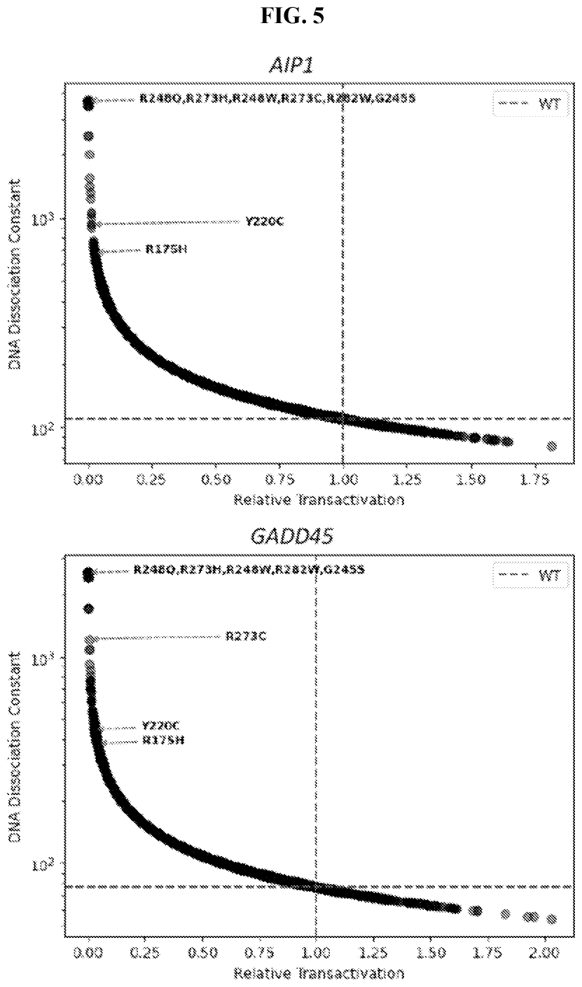

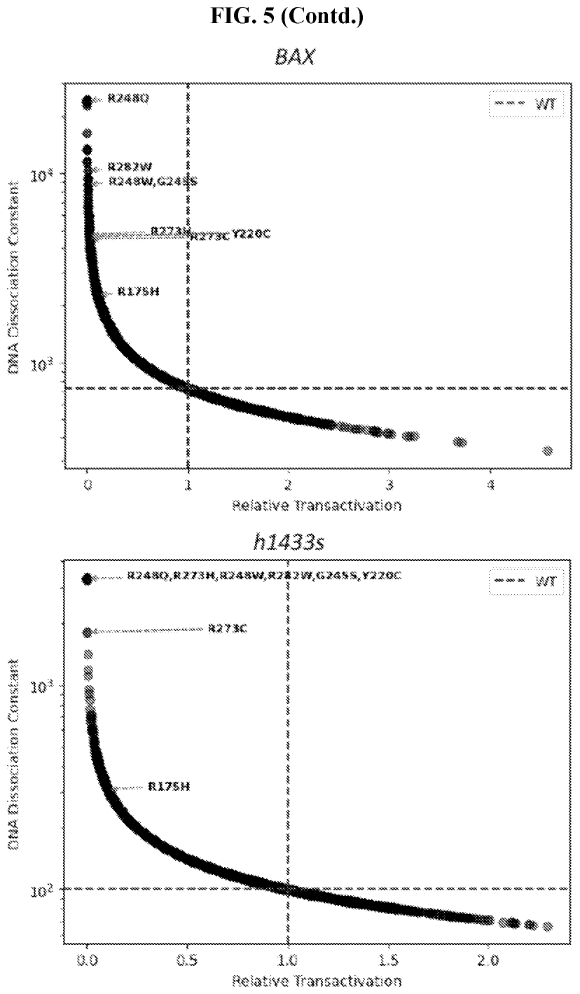

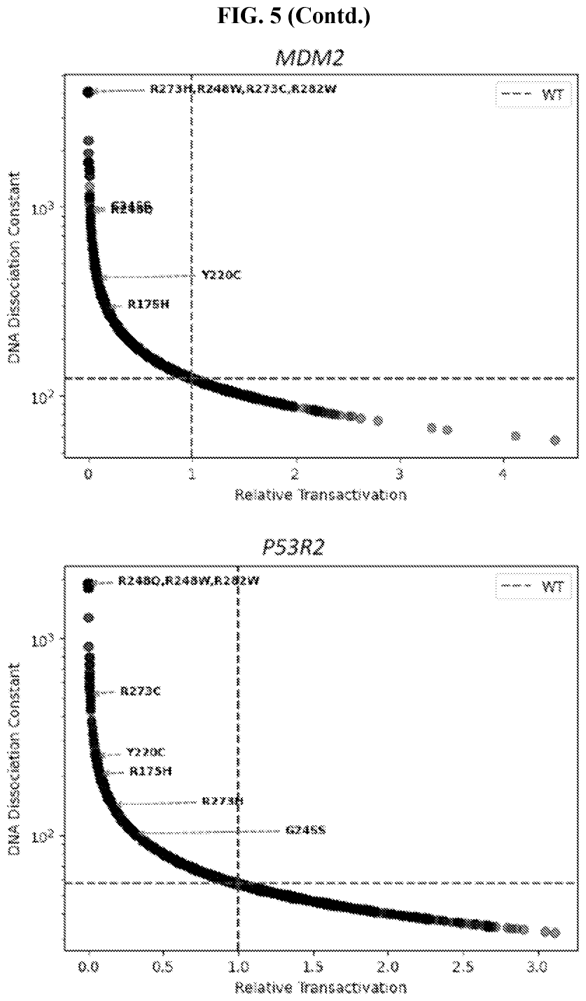

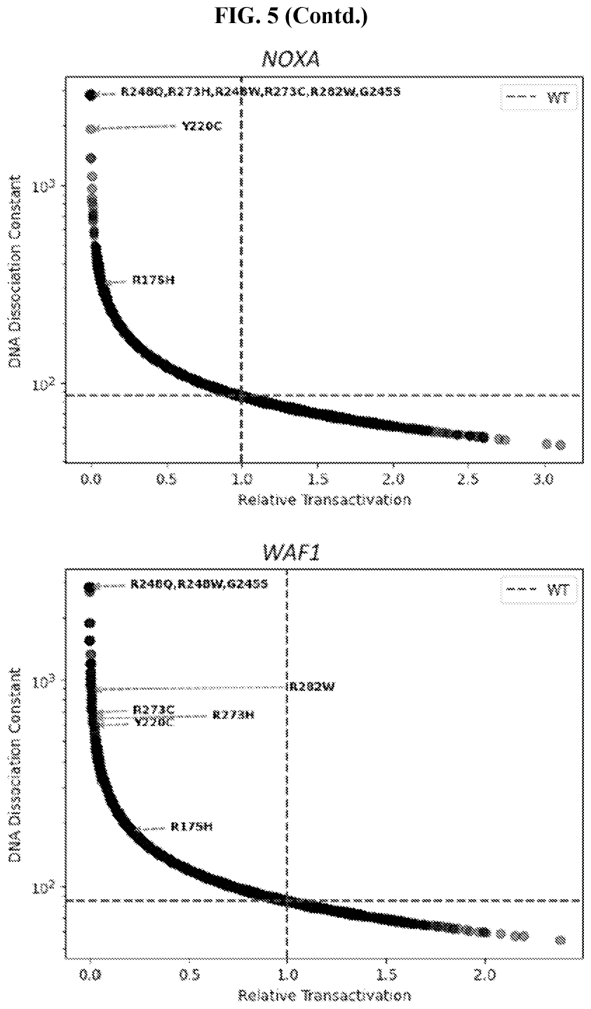

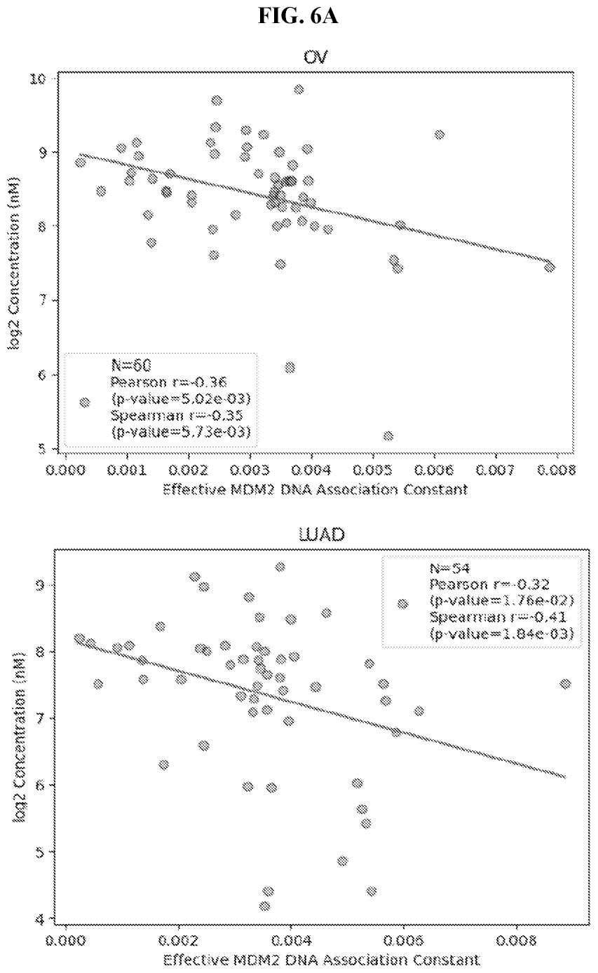

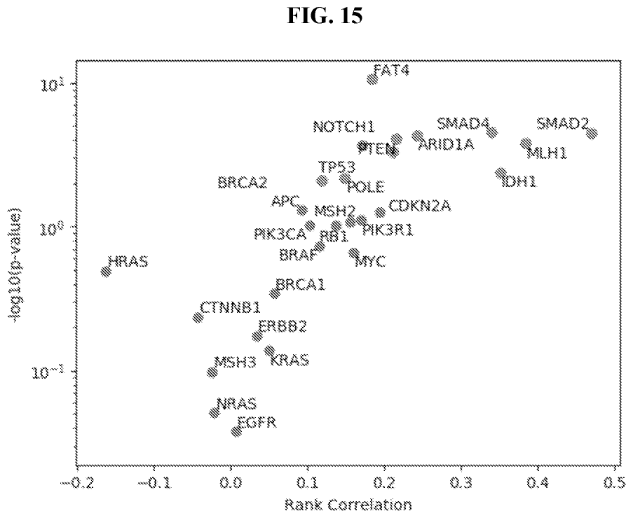

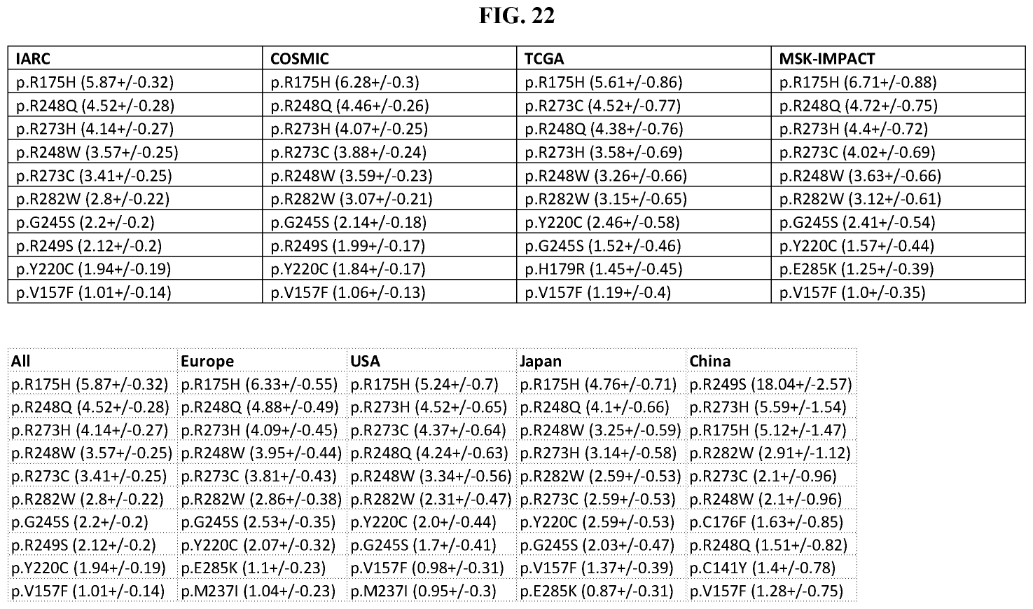

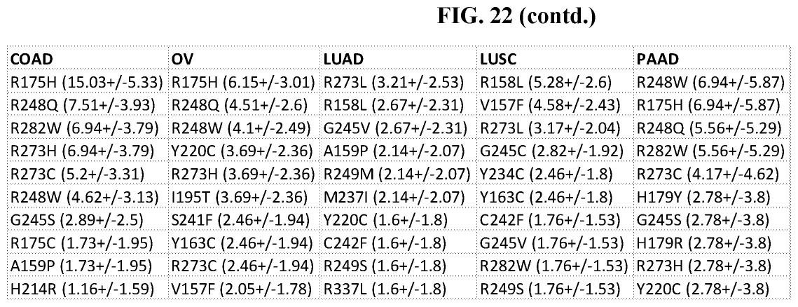

Mutation frequency distributions for commonly mutated driver genes are conserved across multiple cancer mutation databases (FIGS. 1A-1B), and innate mutation rates based on trinucleotide context significantly correlated with mutation frequencies for several genes (FIG. 15). Amino acid conservation over homologous proteins, a proxy for functional phenotype (FIG. 1C), and in silico predicted likelihood of presentation by major histocompatibility complex class-I (MHC-I) molecules (FIG. 1D) across driver genes, finding hotspot mutations create neoantigens less likely to be presented than non-hotspots (Marty, R. et al. Cell 171, 1272-1283.e15 (2017)). Several genes, particularly the tumor suppressors TP53 and PTEN, have hotspots which optimize conservation and immunogenicity (FIG. 1E), implying fitness may be driven by both features. The Examples disclosed herein focus on TP53 as it is widely mutated in tumors, with well-established pan-cancer hotspots whose order is conserved across databases (FIG. 1B, FIG. 22), and thorough functional data is available (Kato, S. et al. Proceedings of the National Academy of Sciences 100, 8424 (2003)). Altered transcription factor function of mutant p53 was quantified across eight principal transcriptional targets by leveraging a quantitative yeast assay (FIG. 1F, FIG. 5). While there was a general loss of transactivation for hotspot mutations, many non-hotspots had comparably low transactivation capacity. Moreover, germline class-I MHC molecules present the set of nonamer neopeptides surrounding hotspot mutations worse than non-hotspot peptides across TCGA haplotypes (p-value 4.748e-7, two-sided Welch's T-test; FIG. 1G). Mutant p53 loss of transcriptional affinity and predicted MHC-I binding affinity of derived neopeptides only showed a weak correlation (Pearson r=0.073, p-value 0.117; Spearman r=0.144, p-value 0.002; FIG. 1H). We therefore concluded the mechanisms proposed to underlie mutant p53 fitness all provide predictive information.

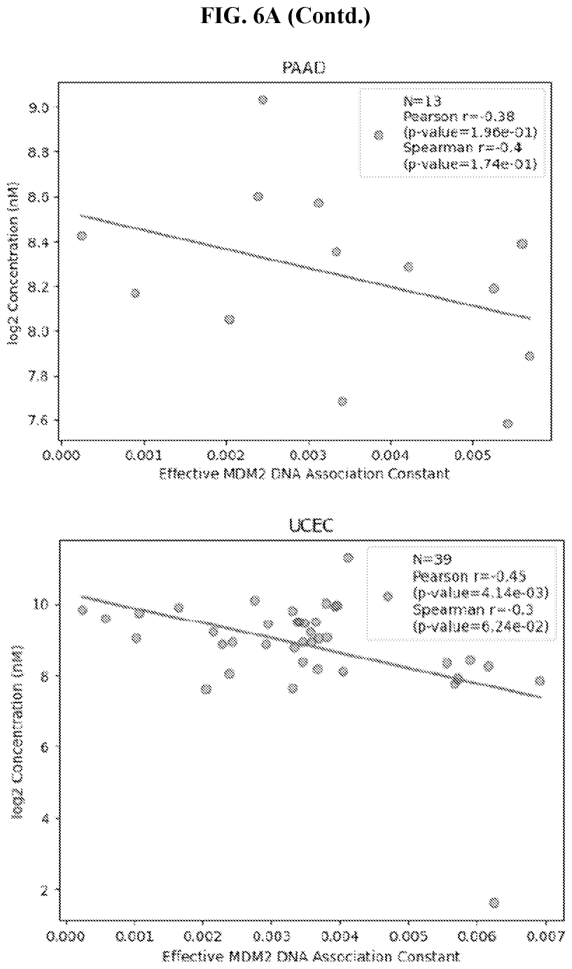

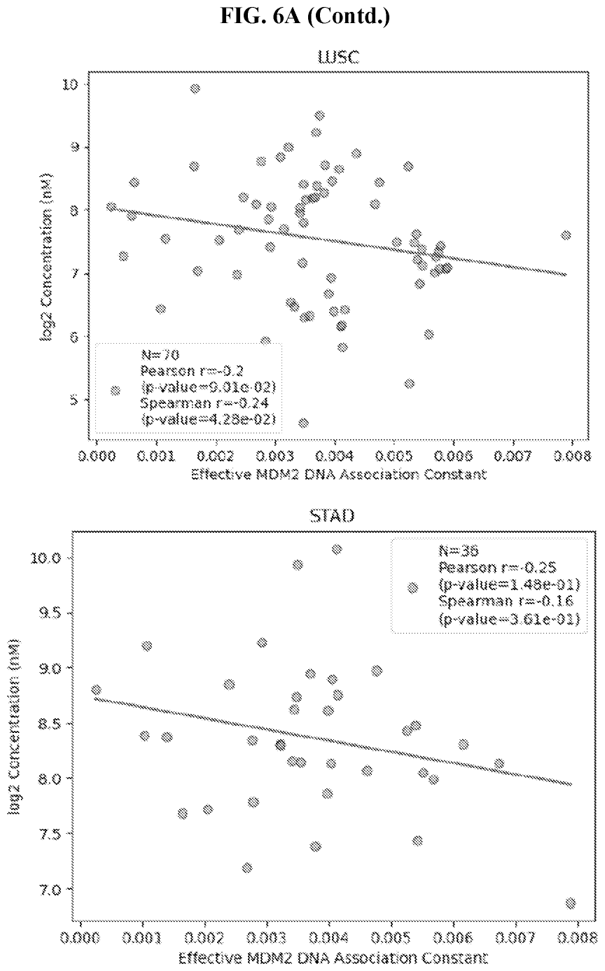

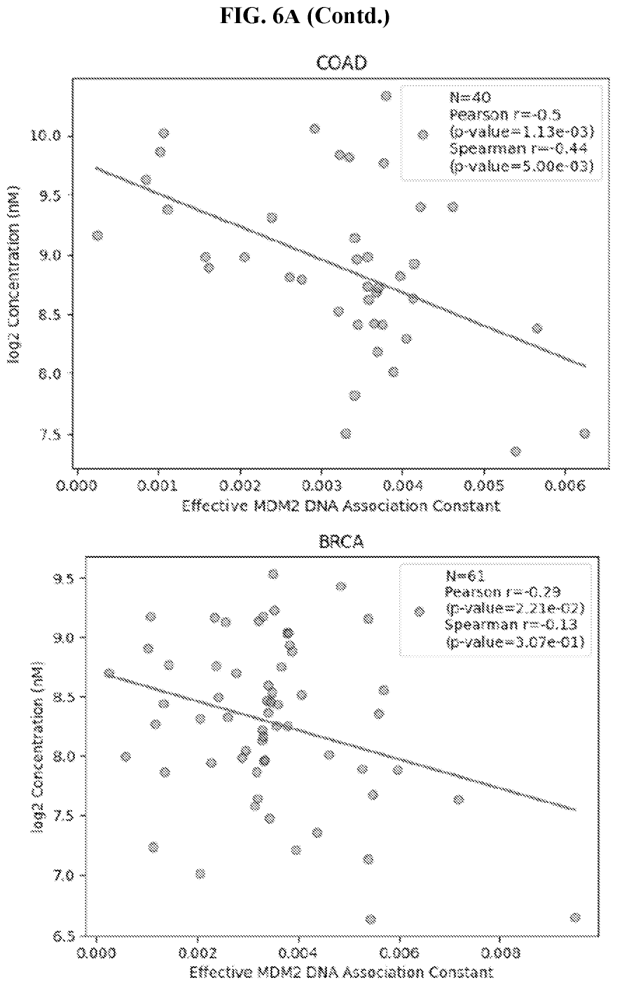

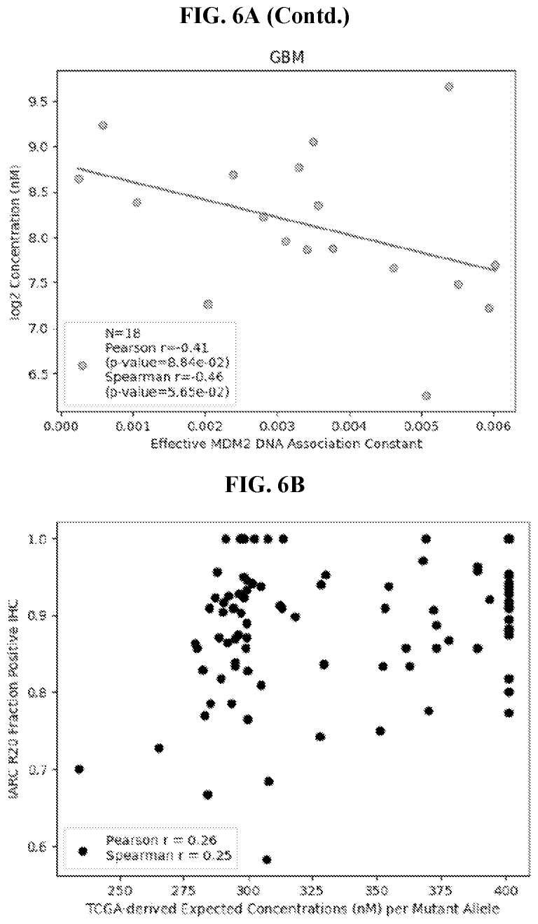

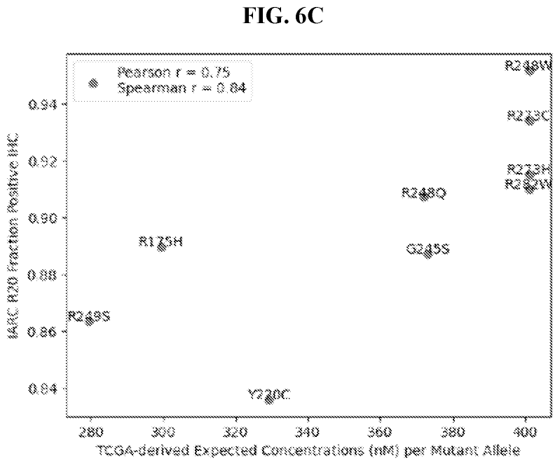

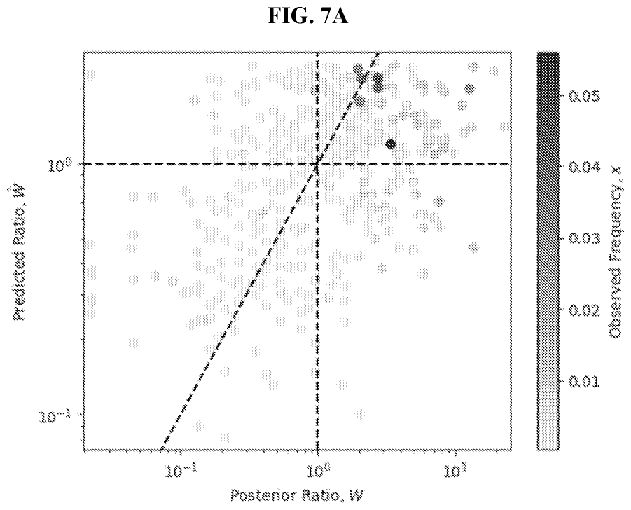

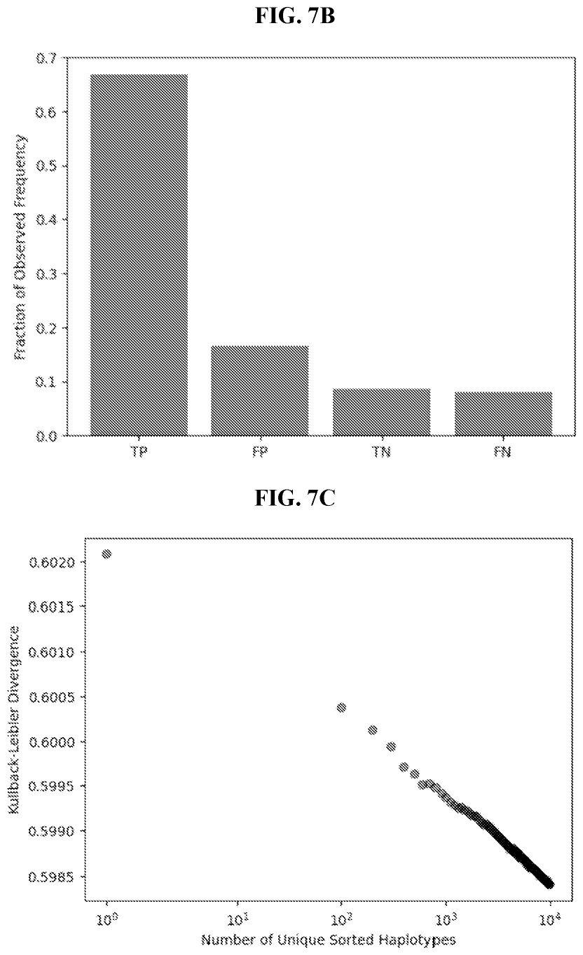

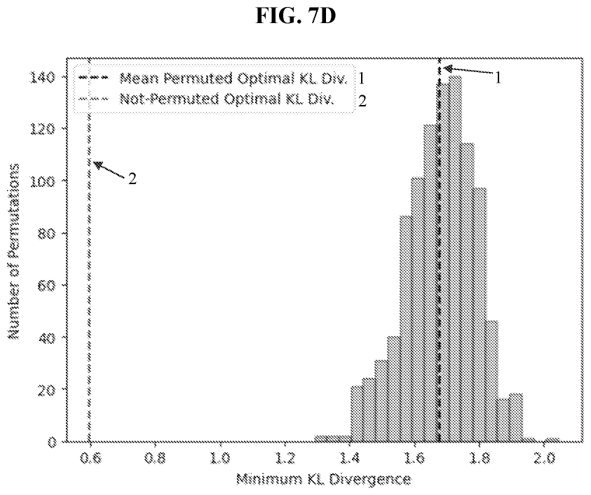

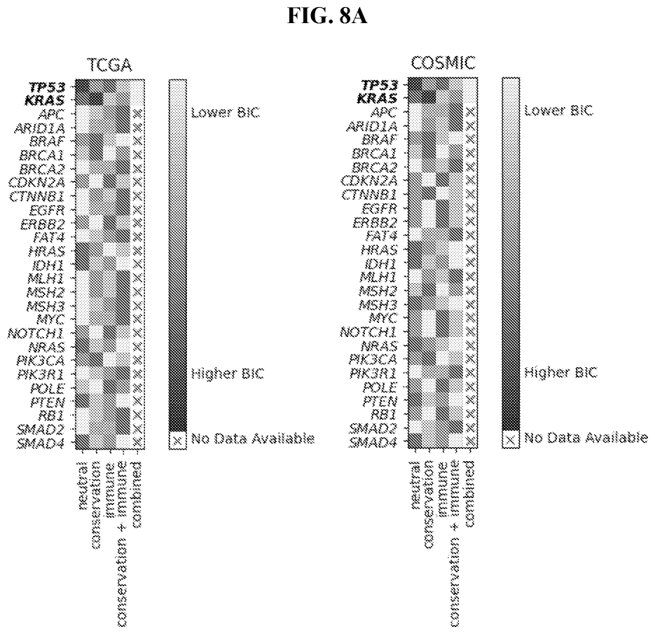

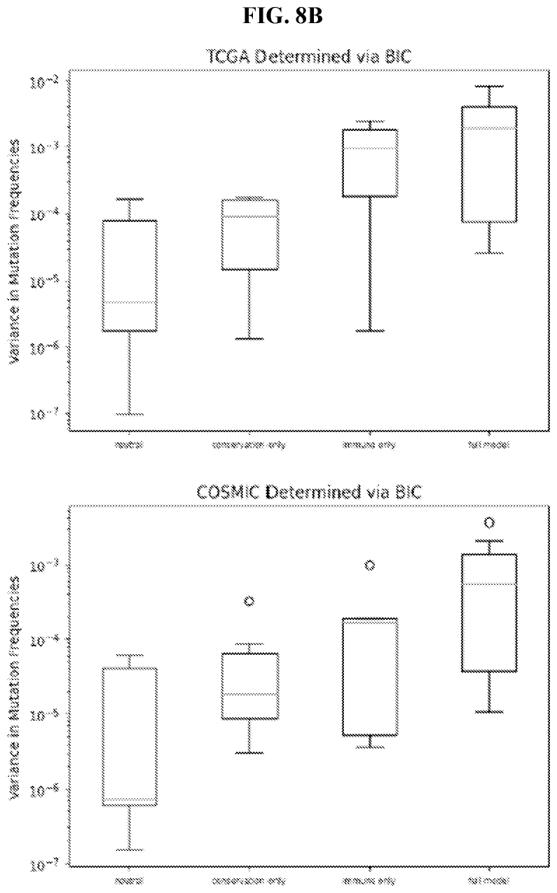

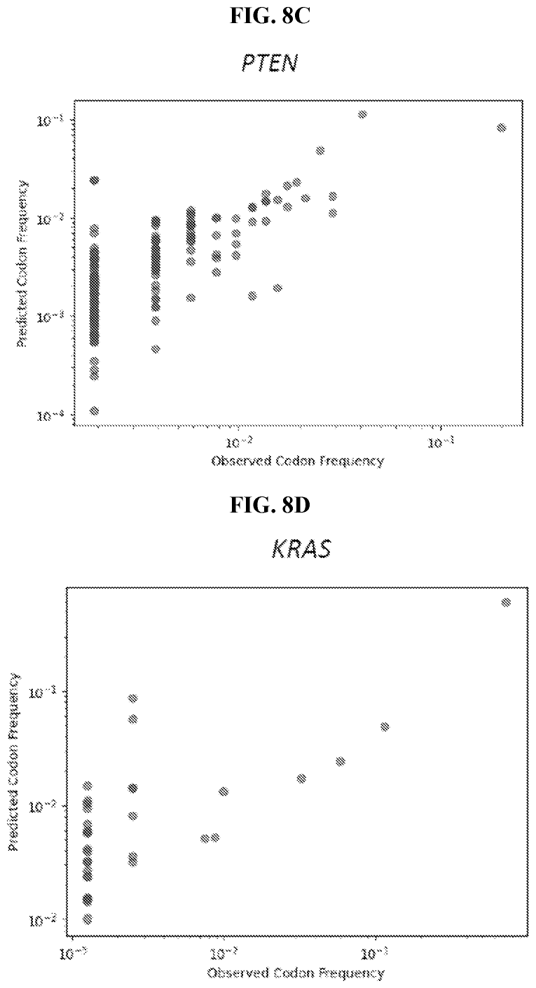

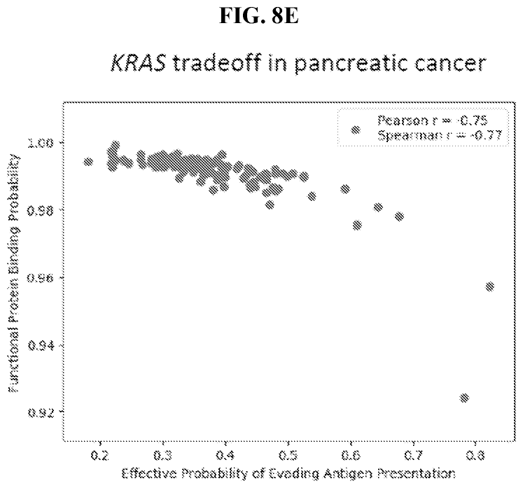

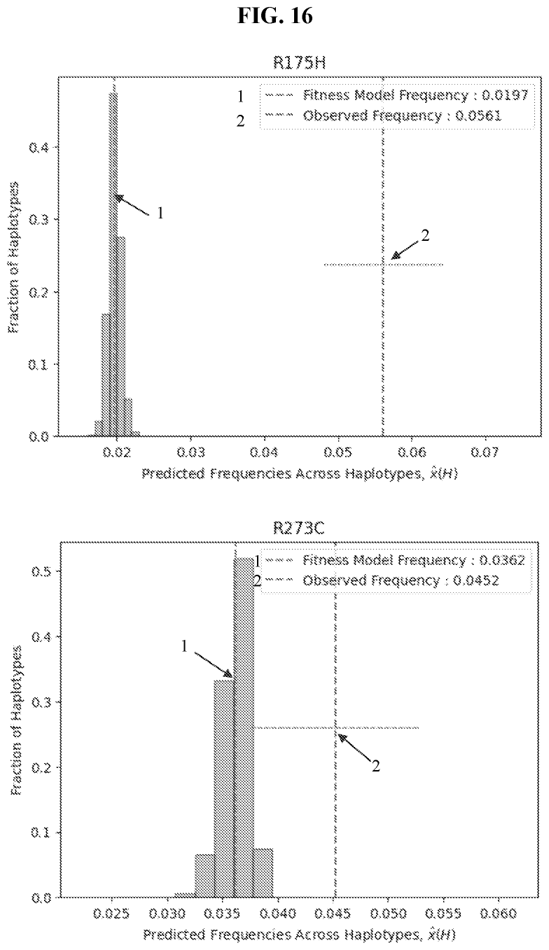

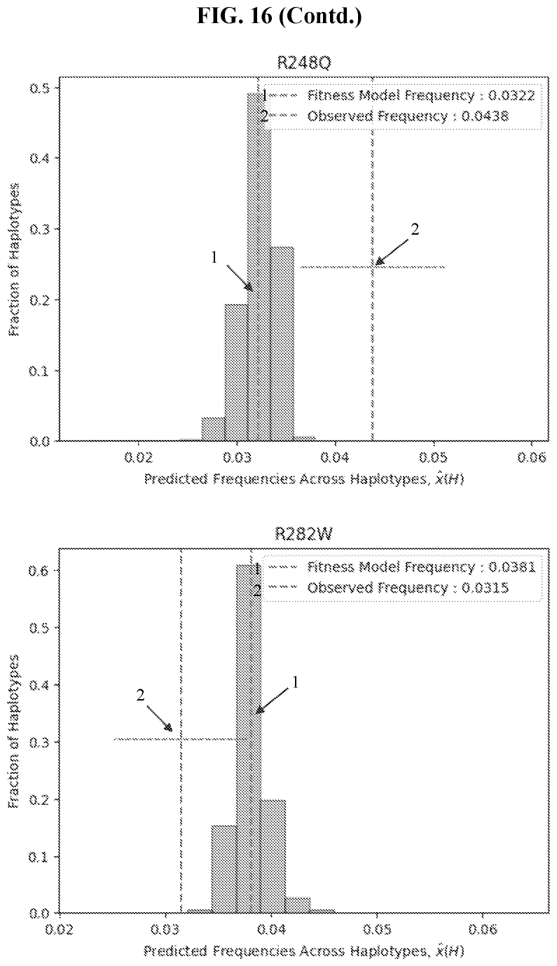

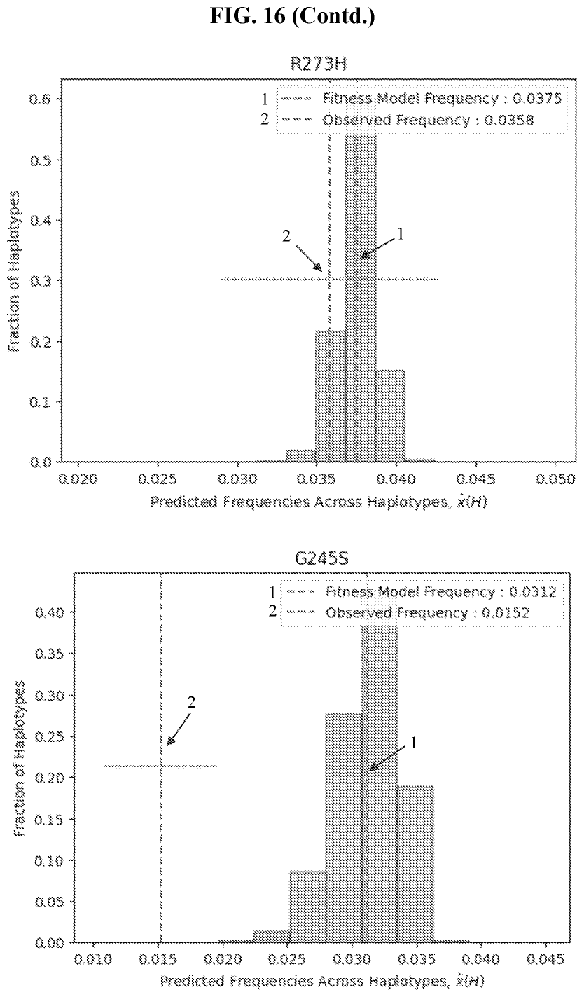

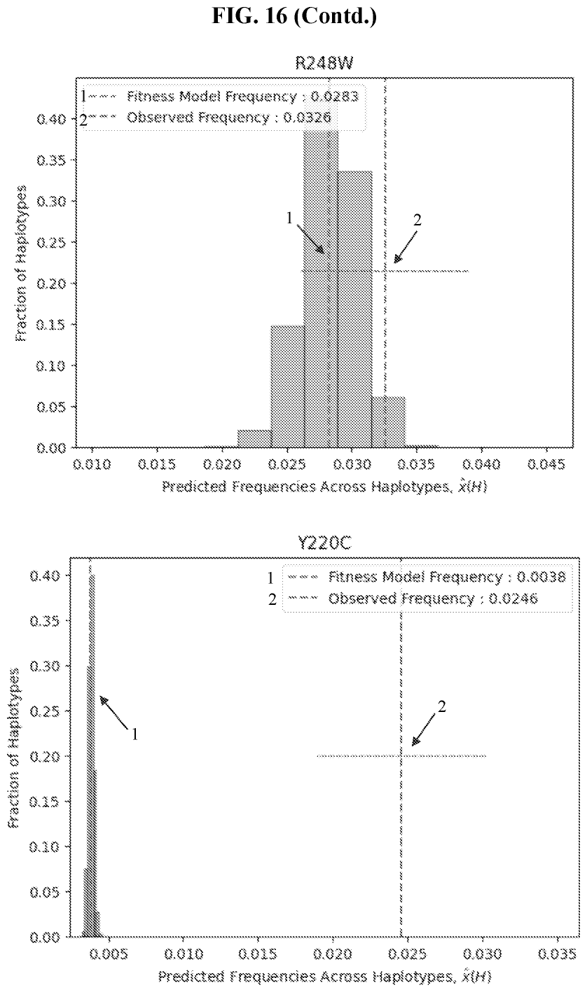

The next objective was to integrate and harmonize this proposed set of features within a mechanistic mathematical model of mutant p53 fitness (Eigen, M. Naturwissenschaften 58, 465-523 (1971); Gerland, U. & Hwa, T. Journal of Molecular Evolution 55, 386-400 (2002); Luksza, M. & Lässig, M. Nature 507, 57-61 (2014); Łuksza, M. et al. Nature 551, 517-520 (2017)). A model based on background mutation rates alone reliably identified hotspots as among the most-frequent (FIG. 2A) but was insufficient to separate hotspots from other mutations. The next objective was to capture variation in mutant p53 concentration, which affects both transcription factor function and neoantigen presentation. Quantitative p53 concentration data is unavailable for most mutants, so TCGA samples were assigned a normalized p53 protein concentration and effective MDM2 promoter affinity (Ma, L. et al. Proceedings of the National Academy of Sciences 102, 14266 (2005); Gaglia, G., Guan, Y., Shah, J. V. & Lahav, G. Proceedings of the National Academy of Sciences 110, 15497 (2013)). Consistently, a significant inverse relationship was found between these two variables across tumor types (FIG. 2B, FIG. 6A), and significant correlation between p53 concentration estimates and immunohistochemistry data (FIGS. 6B-6C). A non-linear two-parameter model that separates p53 fitness onto a positive (pro-oncogenic) and a negative (immunogenic) component was constructed (Methods). For each variant, the positive advantage was quantified as the median probability of “not-binding” target promoter DNA sequences and the negative cost was quantified as the geometric mean of the probability each mutant-derived peptide would bind each MHC-I allele molecule within the germline haplotype. These fitness components couple to one another via mutant p53 concentration. Each component is given an appropriate weight by minimizing the Kullback-Leibler divergence of the predicted frequencies with respect to the observed frequencies in TCGA. The fitness model disclosed herein successfully predicts the overall mutation distribution, both per mutation and per codon (FIGS. 2C-2D, FIG. 16); differentiates hotspot mutations from the bulk; and accurately predicts the increase or decrease in each mutant frequency with respect to the background mutation frequencies in 69.36% and 64.78% of mutants, respectively (FIGS. 7A-7B). Explaining the distribution of TP53 mutations requires both functional and immune components via determination of model relative likelihoods, with the functional component carrying greater weight (FIG. 23, Methods). Model optimization strongly depends on sampled MHC-I haplotype space (FIG. 7C, FIG. 16) and all mutant phenotypes (Methods, FIG. 7D). A similar model was optimized and applied to multiple driver genes, with conservation used as a proxy for function as these genes lacked precision measurements for their mutant phenotype (FIG. 8A, Methods). Combined models were more predictive for mutation distributions with larger frequency variance, implying increased mutation frequency variance relates to increased selection for certain mutations as expected from Fisher's Theorem (FIG. 8B). As examples, a combined model worked particularly well to predict the PTEN mutation distribution (FIG. 8C). For KRAS, we were able to include available binding affinities to downstream RAF effector protein for hotspots to quantify positive functional fitness (Hunter, J. C. et al. Molecular Cancer Research 13, 1325-1335 (2015)), in addition to inferred conservation and immunogenicity, to build a highly predictive model (FIG. 8D, Methods).

To represent the landscape of mutant driver fitness, we defined the “free fitness” of each mutation as the sum of the positive functional fitness, the negative immune fitness, and the logarithm of the background frequency (Methods). The free fitness landscape (FIG. 2E) was plotted and a general trade-off was observed between intrinsic terms (background frequency and functional fitness) and extrinsic immune terms (Pearson r=−0.31, p-value <0.0001; Spearman r=−0.33, p-value <0.0001). The trade-off observed in p53 is reminiscent of other evolutionary trade-offs, and it was hypothesized that TP53 hotspots are nearly Pareto optimal (Shoval, O. et al. Science 336, 1157-1160 (2012); Pinheiro, F., Warsi, O., Andersson, D. I. & Lâssig, bioRxiv 2020.07.02.184622 (2020). The Pareto front was computed and the optimal fitness coordinate constrained by the front was identified utilizing our model (FIG. 2E, Methods). TP53 hotspots have the statistically highest free fitness (Welch's T-test p-value <0.0001, FIG. 2F) and occupy an optimal regime nearly on or on the Pareto front. However, there is substantial variation between hotspot mutations. For instance, R175H is functionally the most wild-type-like hotspot but typically has the poorest MHC-I binding capacity. By contrast, R248Q/W mutations have near complete loss of transcriptional function and therefore can more often “afford” to generate potentially immunogenic neoantigens, since the proliferative competitive advantage induced by this mutation would offset the cost of immunogenicity. Likewise a trade-off between intrinsic and extrinsic free fitness components was found for KRAS hotspots in TCGA pancreatic adenocarcinoma, where KRAS is typically mutated (FIG. 8E).

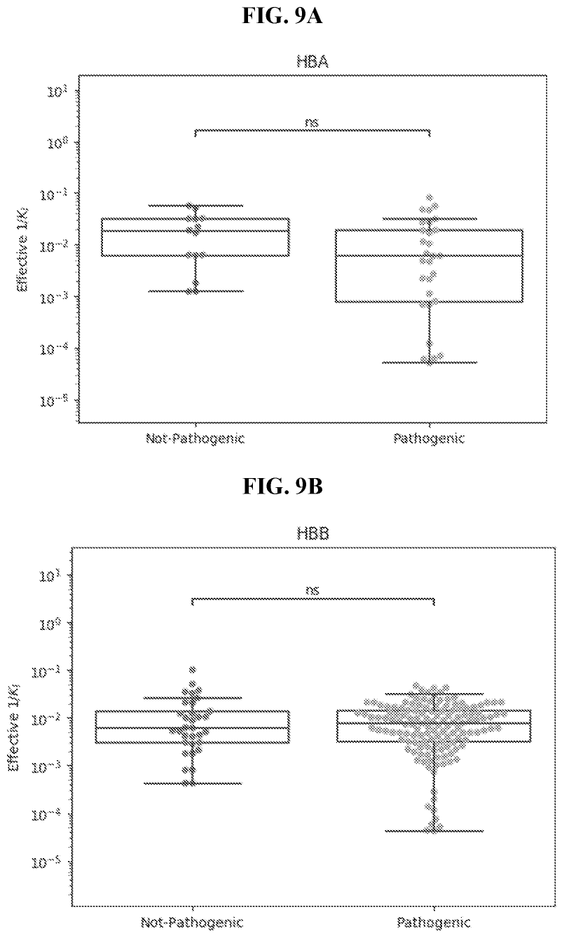

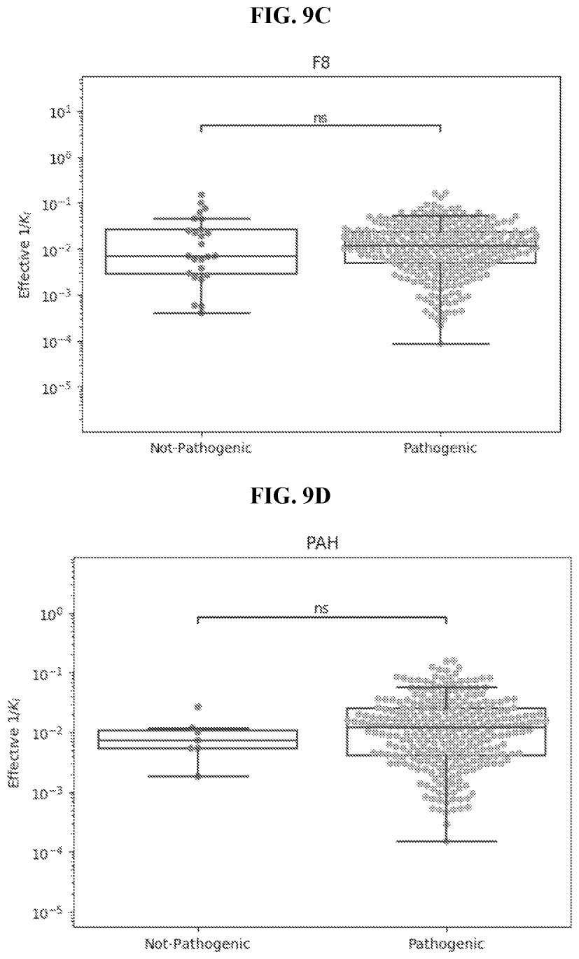

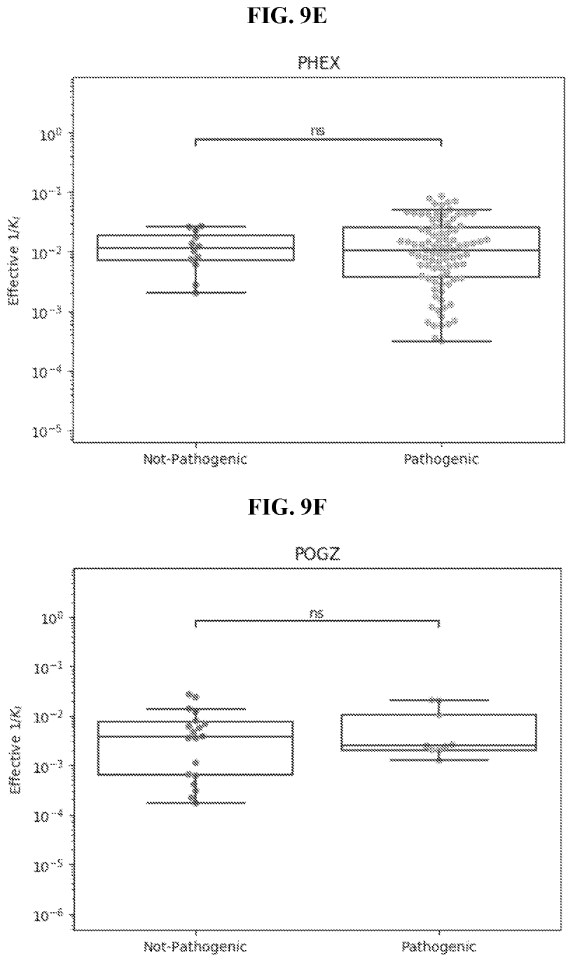

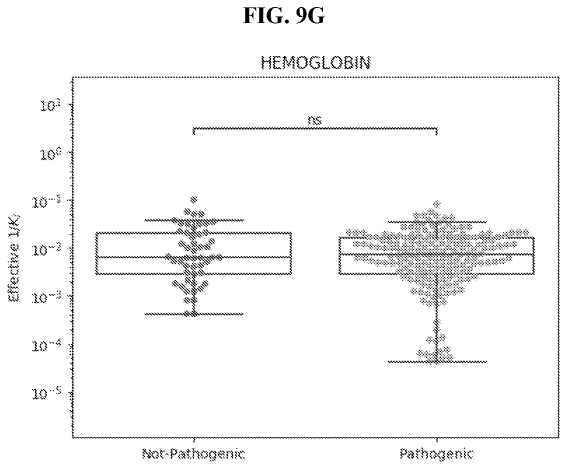

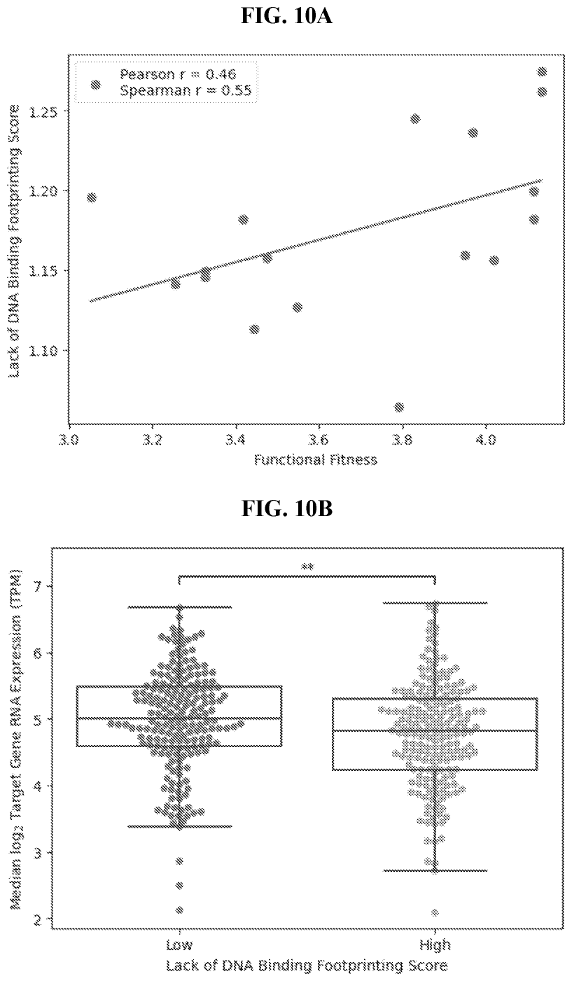

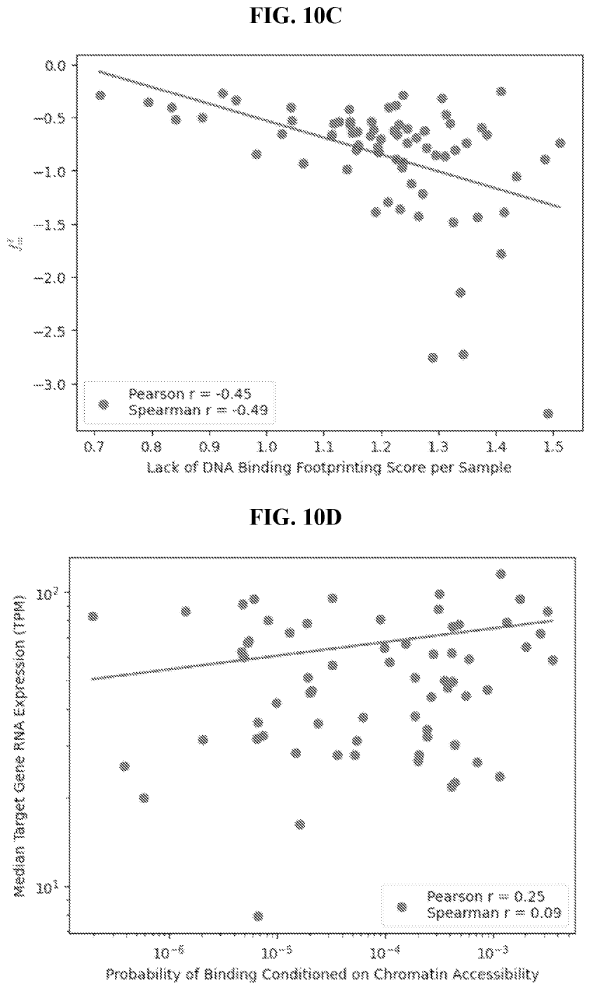

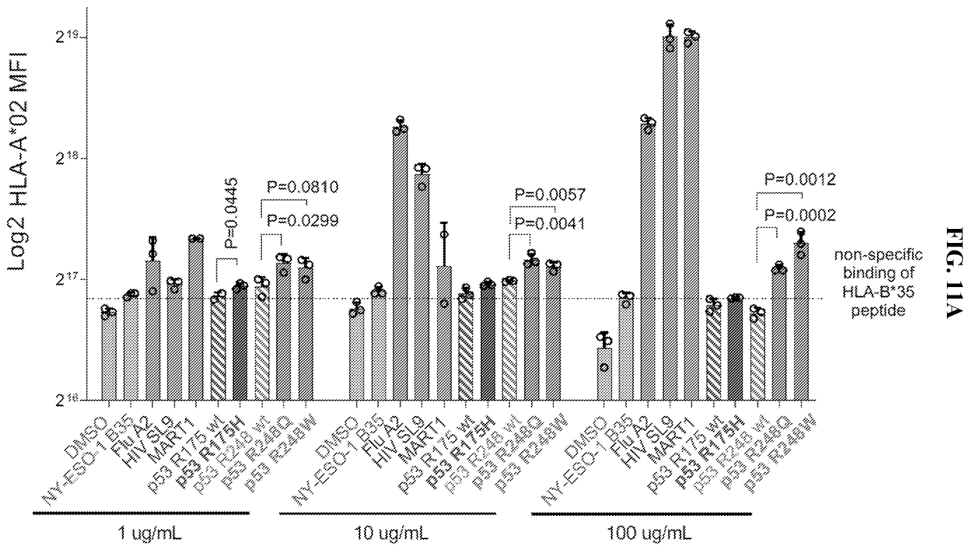

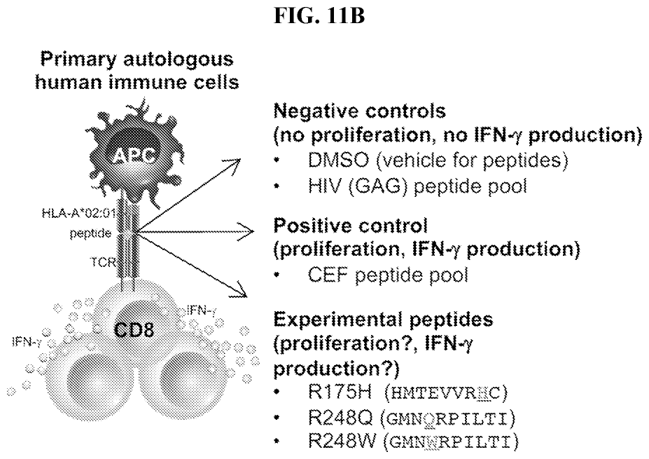

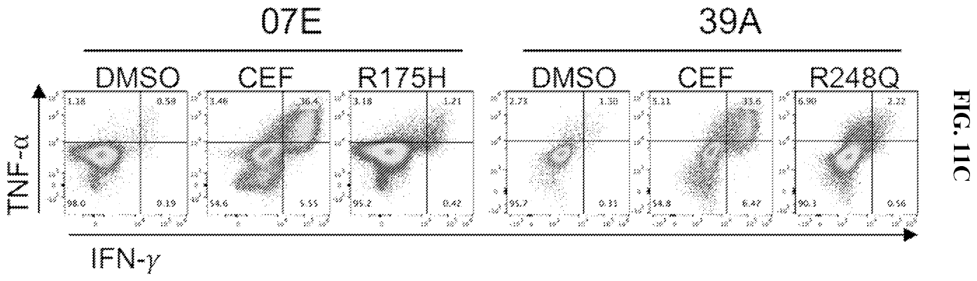

One possible explanation is that mutations across diverse diseases which are more deleterious to protein function are more likely to generate more immunogenic peptides due to lack of amino acid conservation. Non-pathogenic and pathogenic mutations in a curated set of non-cancerous disease driver genes were therefore compared and both types of mutations were found to generate comparably predicted immunogenic peptides (FIG. 9), implying the trade-off observed is not to be expected a priori. As functional predictions for mutant TP53 described herein are based on precision yeast assays with artificial constructs, evidence of an oncogenic-immunogenic trade-off utilizing independent human TCGA ATAC-seq and RNA-seq assays were also checked to develop a score for the lack of transcription factor occupancy in mutant p53 (Methods). The functional component of the TP53 fitness model correlates significantly with lack of binding (FIG. 10A), and samples with increased lack of p53 binding consistently had a concomitant decrease in p53 target gene RNA expression (FIG. 10B). The oncogenic-immunogenic trade-off was independently re-derived by comparing inferred immunogenicity to lack of binding (FIG. 10C). Finally, as a further control, a correlation between the probability of DNA binding used in the fitness model derived from yeast and median target gene RNA expression conditioned on chromatin accessibility was found (FIG. 10D).

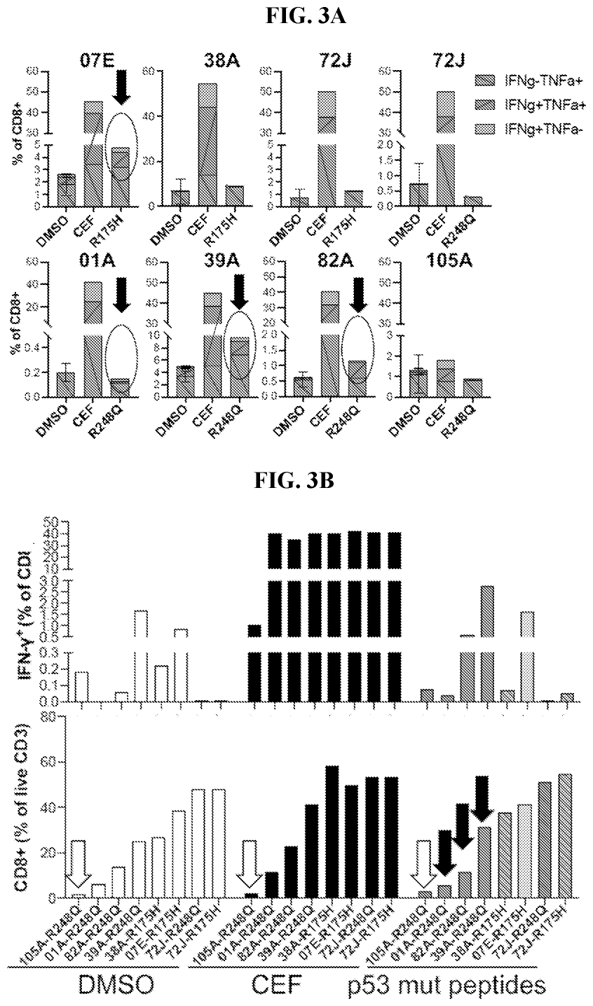

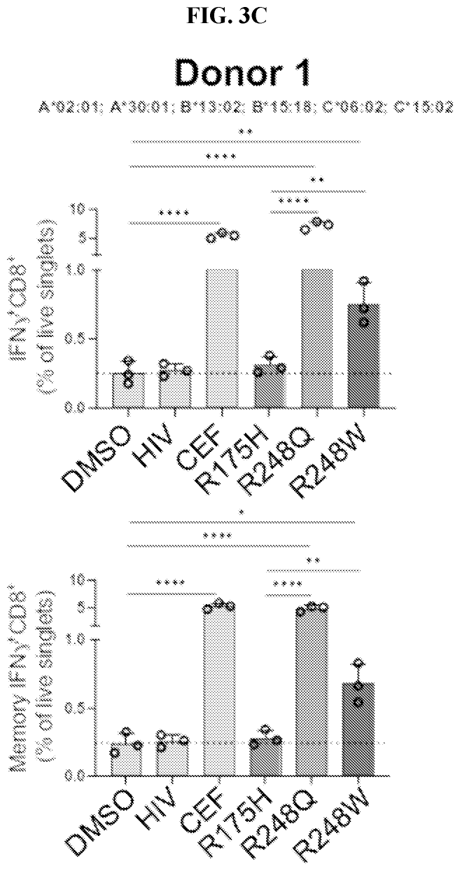

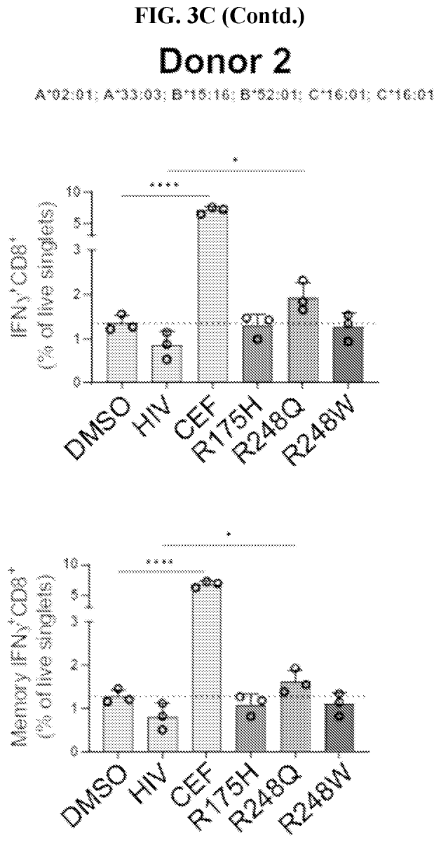

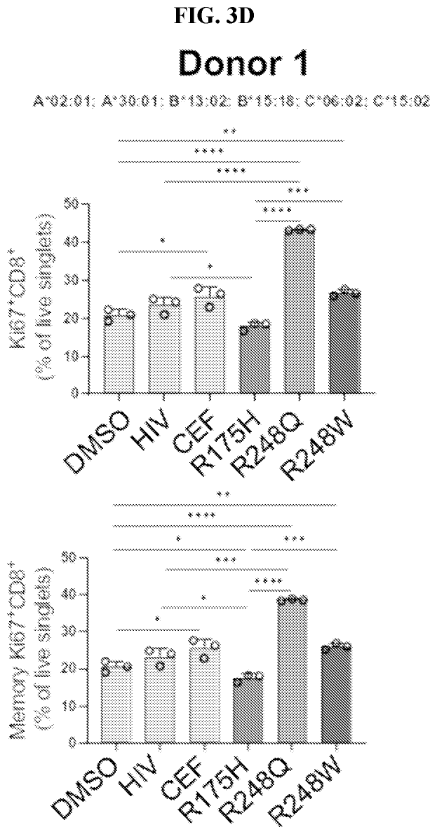

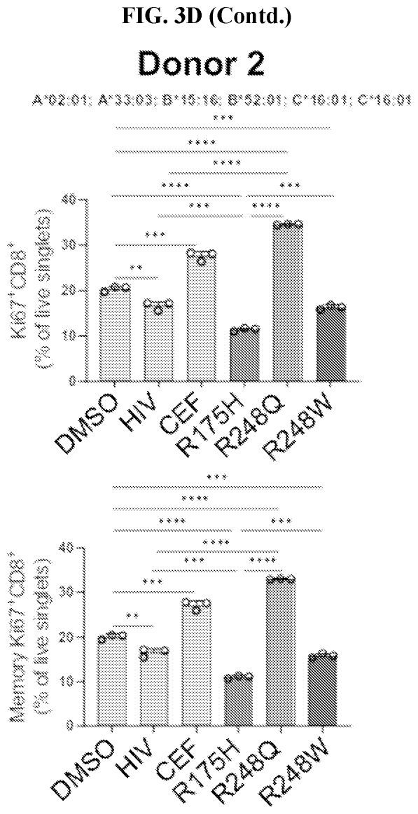

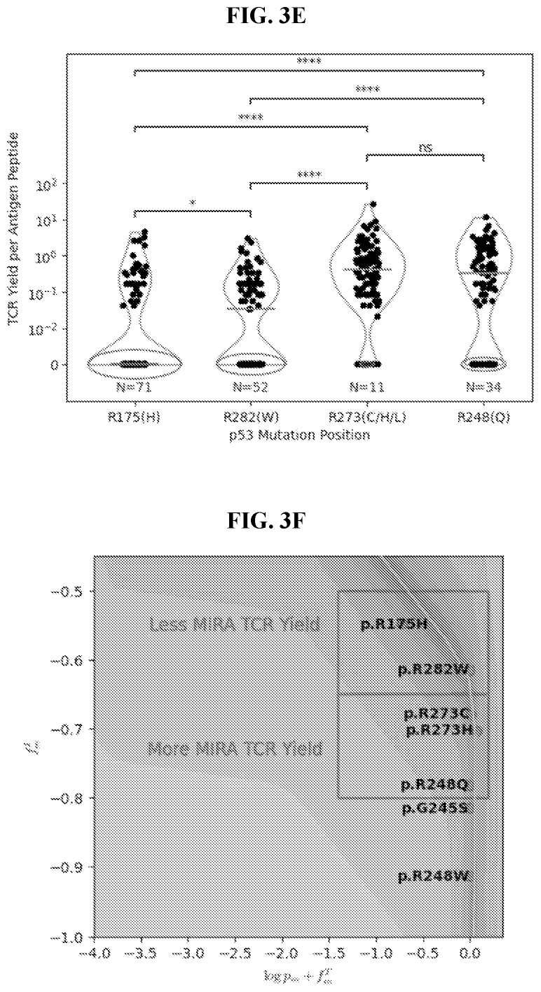

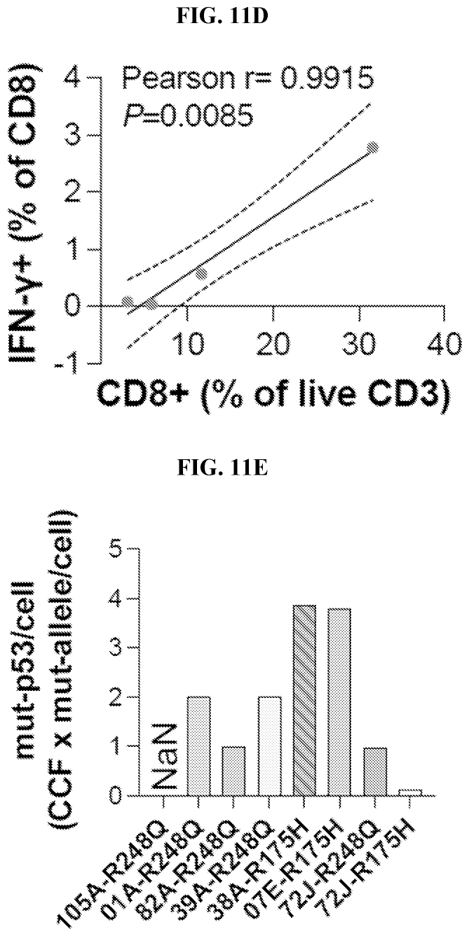

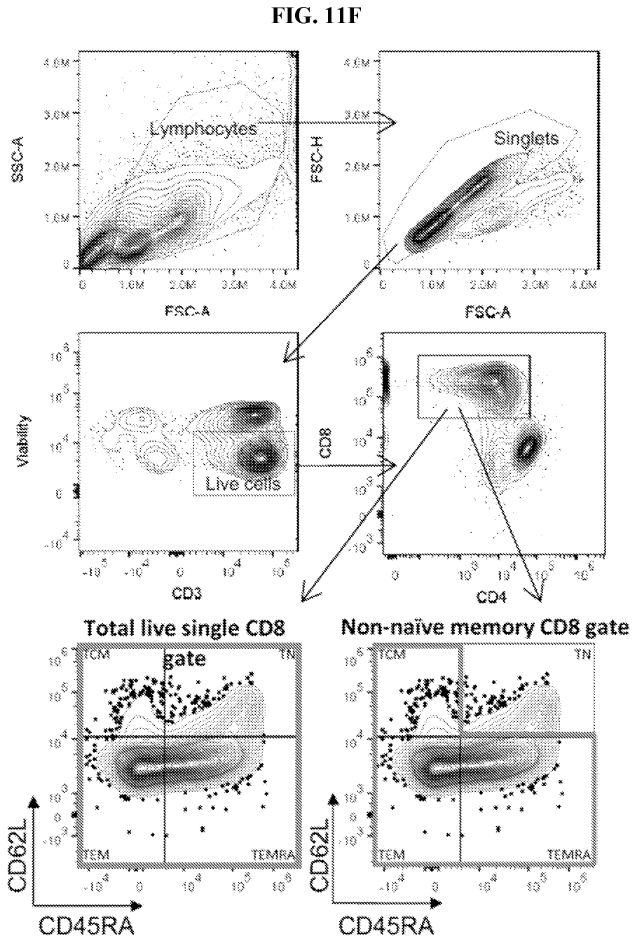

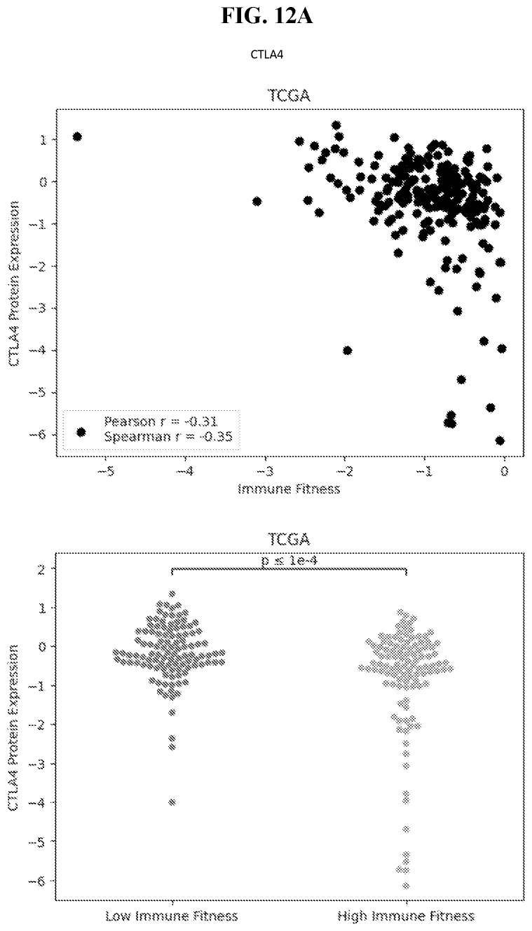

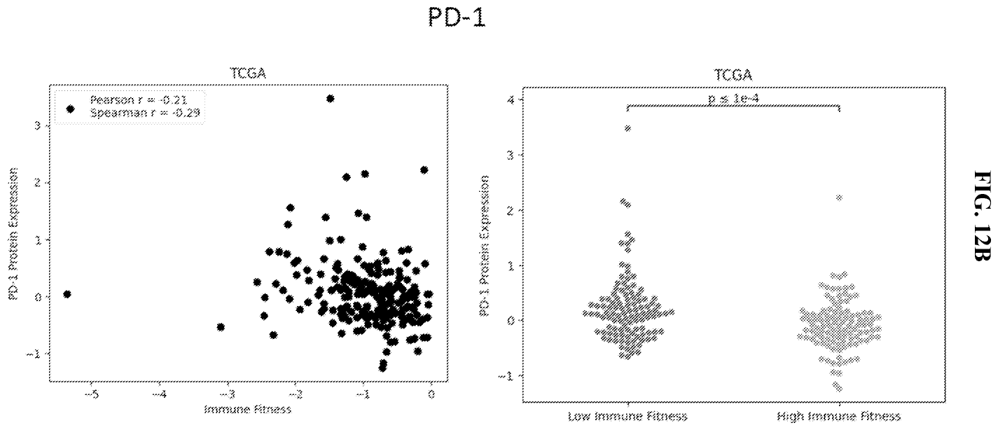

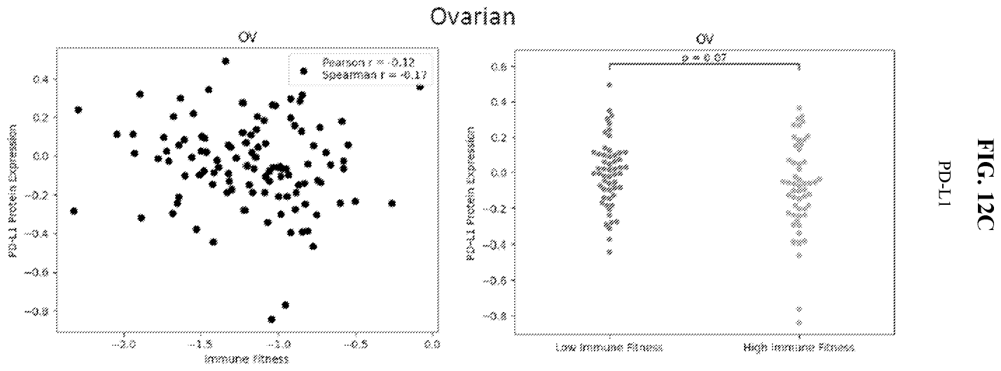

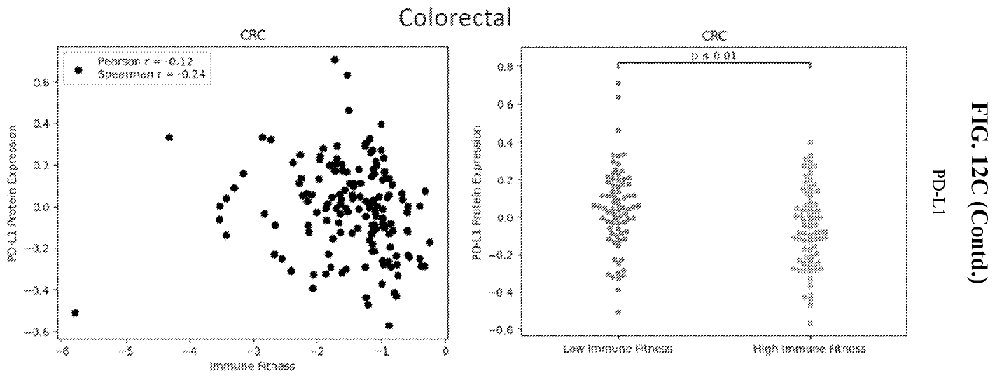

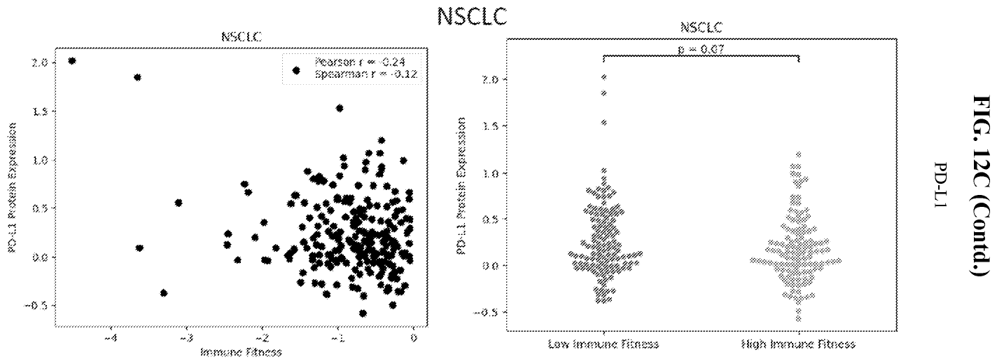

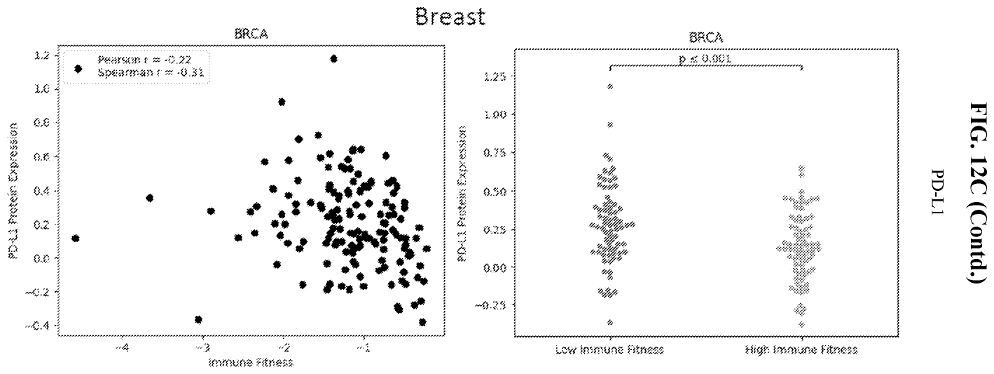

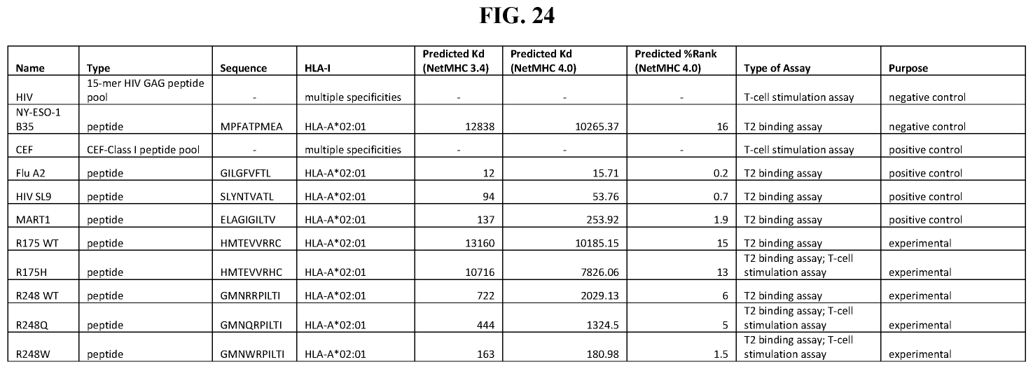

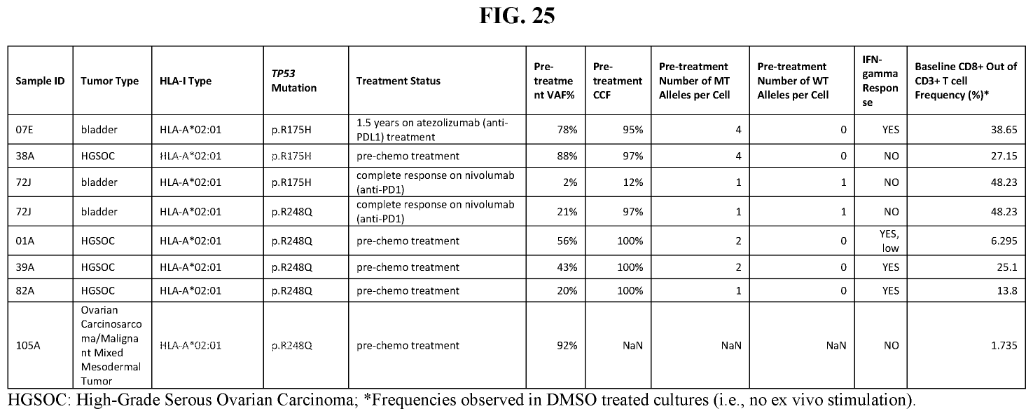

The predictions for mutant p53 were tested using peptides from hotspot mutations predicted to be presented on HLA-A*02:01 (NetMHC 3.4, <500 nM; Methods), the most frequent MHC-I allele in TCGA. Based on this, two 10-mers for each R248Q/W mutation which differed by one amino acid were selected (FIG. 24). In agreement with the model predictions, we were unable to find a R175H peptide that matched the same criteria, so we selected a peptide which was previously reported to bind to HLA-A*02:01²⁸ (FIG. 24). First, to test for the ability of these peptides to bind and stabilize HLA on the cell surface, the peptides were incubated with TAP2-deficient human lymphoblastoid T2 cells. These cells were used to evaluate peptide binding to MHC, as they do not present endogenous peptides and will only express HLA-A*02:01 molecules on their surface if pulsed with peptides that bind MHC. R248W and R248Q peptides but not the R175H peptide were able to significantly increase HLA-A*02:01 expression on T2 cells in a dose dependent manner in comparison with the respective wild-type peptide sequence, indicative of correct binding and stabilization of the complex on the cell surface (FIG. 11A; FIG. 24). The ability of R175H and R248Q/W TP53 hotspot mutations to elicit differential immune responses in cancer patients in vivo was tested. Seven HLA-A*02:01 patients with tumors carrying those mutations and available peripheral blood mononuclear cell (PBMC) samples at Memorial Sloan Kettering Cancer Center (MSK) were identified. In total, samples in bladder and ovarian cancer from three patients with a somatic R175H mutation (07E, 38A and 72J) and five patients with R248Q mutant tumors (72J, 01A, 39A, 82A and 105A) (FIG. 25) were acquired. One patient (72J) shared both mutations, although the R175H clonal fraction was far lower, and all ovarian patients lost the wild-type allele (FIG. 25). All but two patients (72J and 07E) were immunotherapy-naïve at the time of sample collection. Patient 72J, who had a tumor with both hotspot mutations, had an on-going complete response to nivolumab (anti-PD1) treatment with no disease detectable at the time of PBMC collection. Patient 07E, who harbored the R175H mutation, was on atezolizumab (anti-PD-L1) treatment at the time of PBMC collection. All other samples (n=1/3 from R175H mutant patients and n=4/5 from R248Q mutant patients) were collected before treatment initiation. PBMC samples were stimulated with the same peptides from the R175H or R248Q mutations or CEF peptide pool or DMSO as positive and negative controls, respectively (FIG. 24), before measuring IFN-γ and TNF-α responses in CD8+ T cells by flow cytometry (FIGS. 3A-3B; FIGS. 11B-11D). No reactivity was found in patient 72J, which harbored both hotspot mutations and had a complete response to nivolumab, suggesting that expansion/persistence of the cognate T-cell pools depends on the levels of the mutant protein. Responses were found in three of four remaining R248Q samples, with response in those samples proportional to CD8+ population size (FIG. 3B; FIG. 11D). By contrast, interestingly, one of two remaining patients with R175H with four mutants TP53 alleles and received anti-PD-L1 treatment, had neopeptide reactivity. Their reactivity was presumably due to the combination of a high R175H concentraion and immunotherapy (Patient 07E; FIG. 3A; FIG. 11E).

To determine if differential reactivity to TP53 hotspots was a broad phenomenon in the healthy population, the capacity of R175H and R248Q/W peptides when loaded onto autologous antigen presenting cells to prime and expand specific T cells in two HLA-A*02:01 healthy donors was compared (FIG. 11B; FIG. 24; Methods). Greater IFN-γ and Ki67 expression in T cells stimulated with peptides containing R248 mutations as compared to R175H was consistently observed in both tested donors (FIGS. 3C-3D; FIG. 11F). Furthermore, yield of TP53 hotspot-specific T-cell clones using the MIRA assay was assessed by Adaptive Biotechnologies in 107 healthy donor PBMC samples representing a set of distinct 25 HLA-A, 46 HLA-B, and 20 HLA-C alleles and multiple peptide lengths (Methods). A total of 40 mutant epitopes from R175, R282, R273, and R248 loci covering the top six p53 hotspots were screened for multiple peptide lengths. The distribution of normalized T-cell receptor (TCR) yield per antigen peptide per donor (indicative of specific clonal expansion) was plotted for each hotspot position (FIG. 3E). Notably, the R175 hotspot yielded a statistically lower TCR reactivity per peptide as compared to all other hotspots, having a median value of zero reacting TCRs per peptide. More generally, hotspot reactivity directly corresponded to the model predictions (FIG. 3F). These results indicate MHC-I haplotype and TCR repertoire distributions of the healthy population may be more likely to react to the R248 locus as compared to the R175 locus.

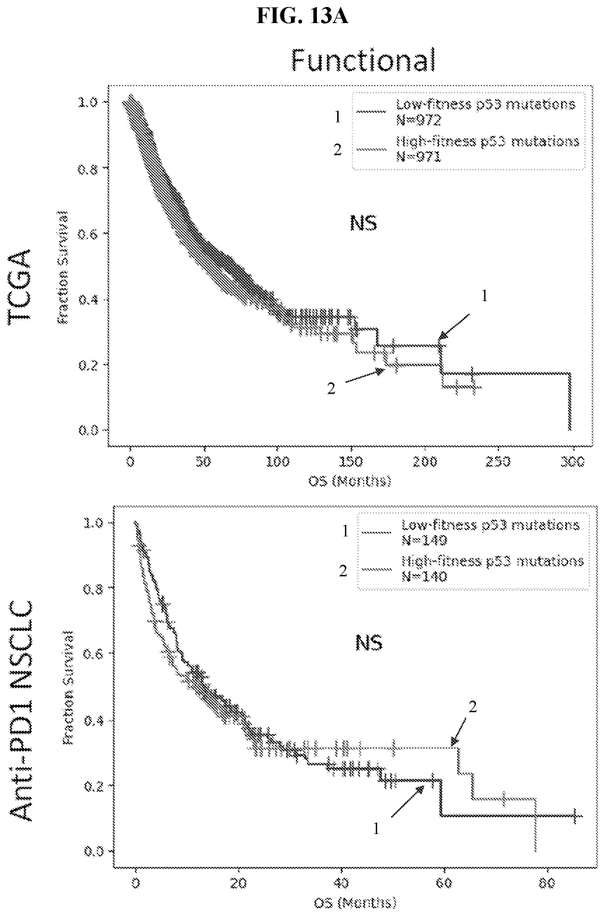

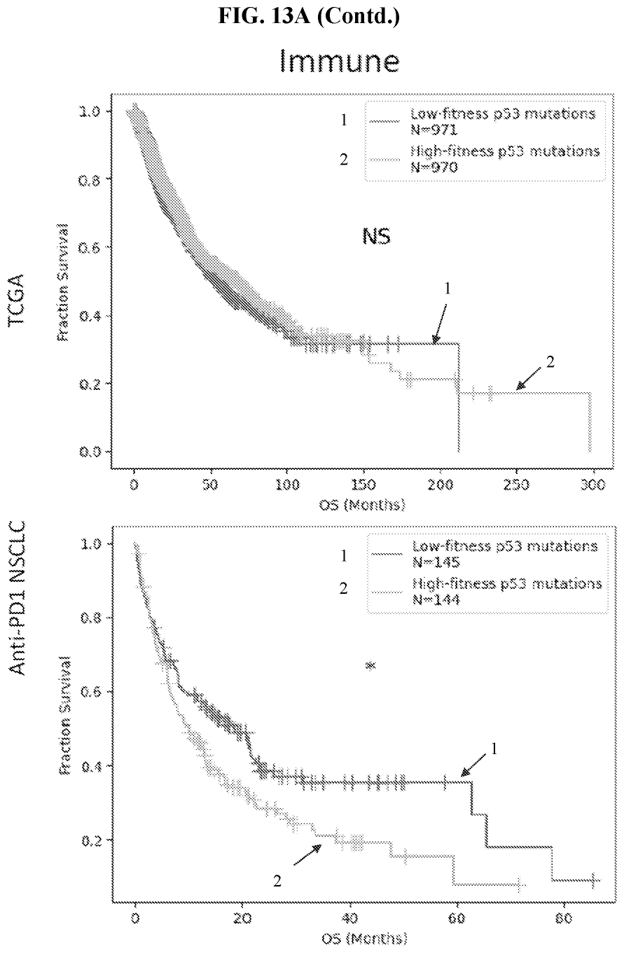

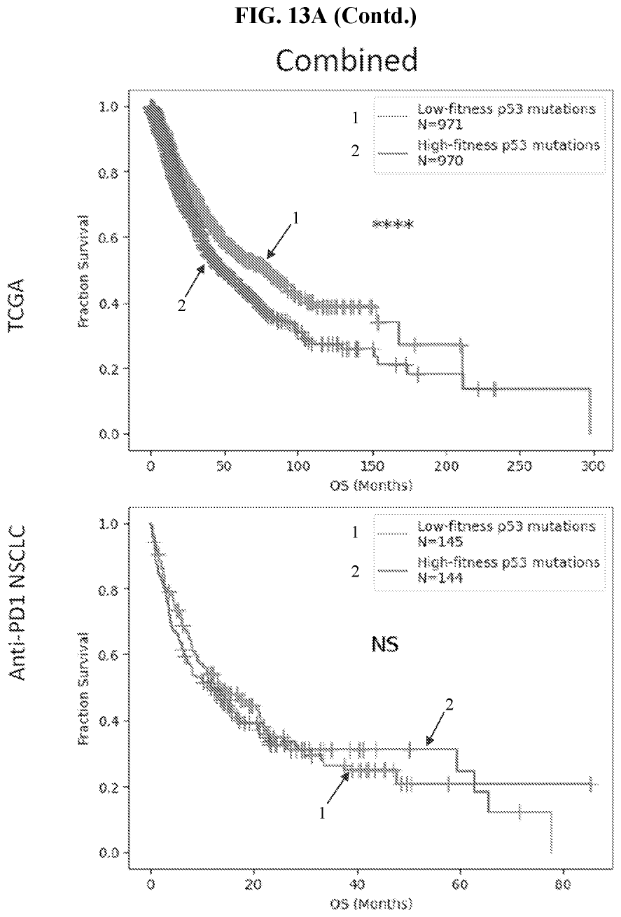

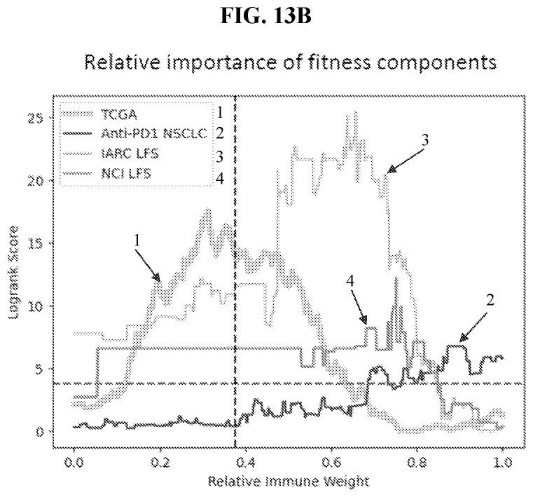

Validating the link between increased immunogenicity and immune response, it was found that expression of immune checkpoint proteins CTLA-4, PD-1 and PD-L1 in TCGA samples with TP53 mutations that had been predicted to be more immunogenic based on their ability to be presented in the germline MHC-I haplotype was increased. This suggests high immune activation and the concurrent establishment of adaptive immune resistance (FIG. 12). When survival based on functional, immune, and total combined fitness in TCGA and anti-PD1 treated patients in a large cohort of non-small cell lung cancer (NSCLC) patients was segregated (FIG. 13A), functional and immune fitness components were required to achieve significant survival separation in TCGA, while immune fitness on its own significantly separated survival in immunotherapy treated NSCLC (FIG. 13A). For robustness, the models were retrained across a range of relative weights between functional and immune fitness (FIG. 13B, Methods), and demonstrated both components contribute to a model optimized for survival separation across TCGA, while the immune component is the main determinant for an equivalent model in the immunotherapy-treated NSCLC cohort.

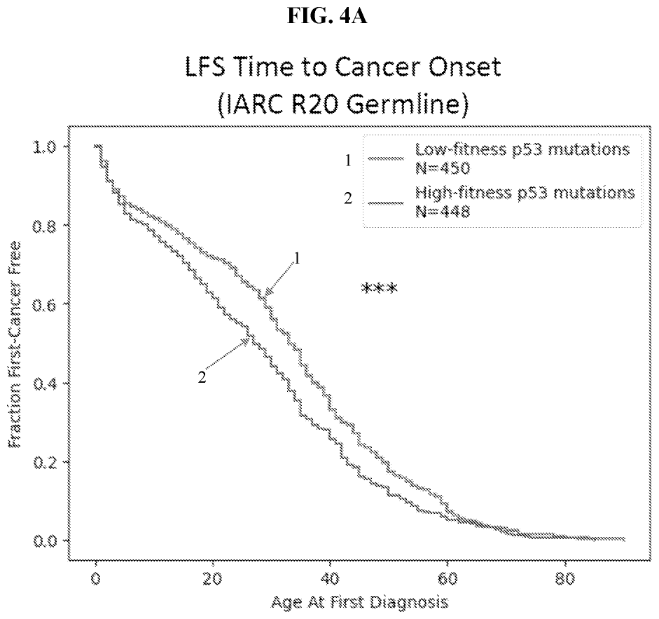

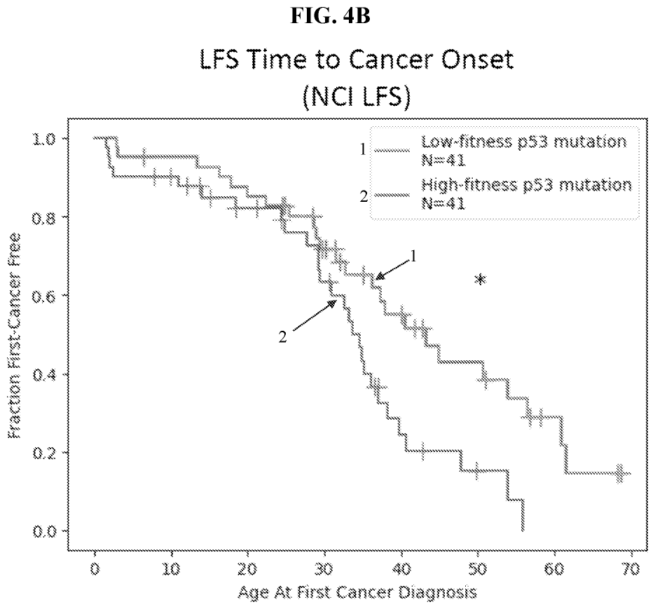

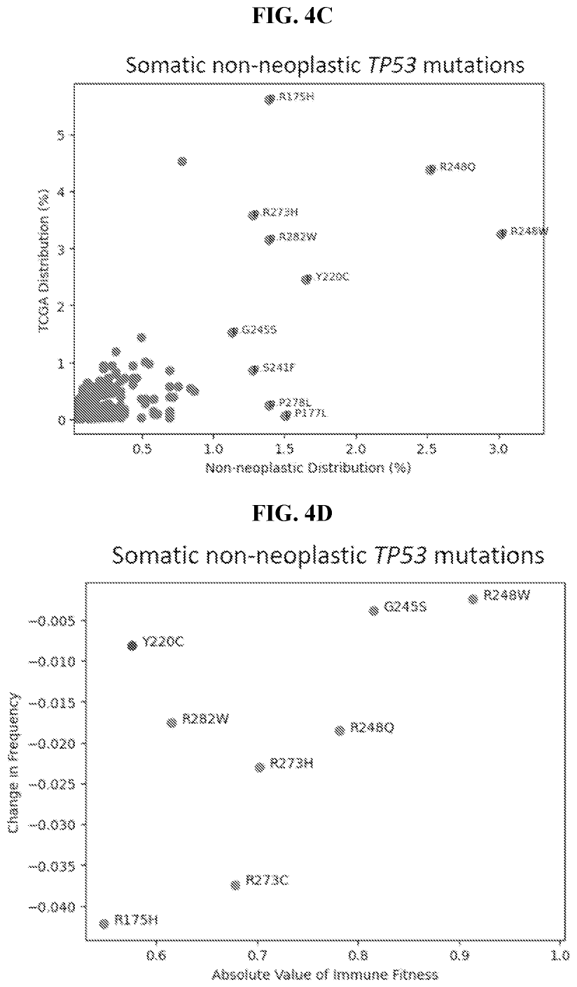

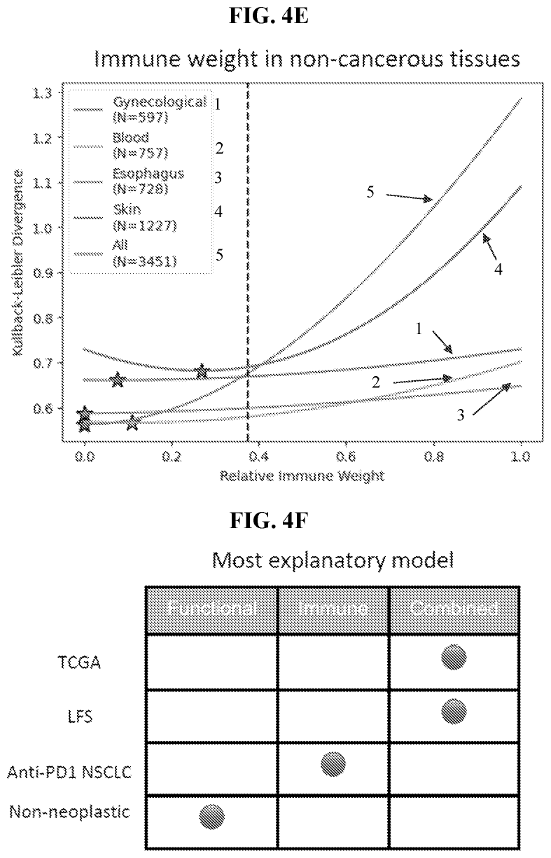

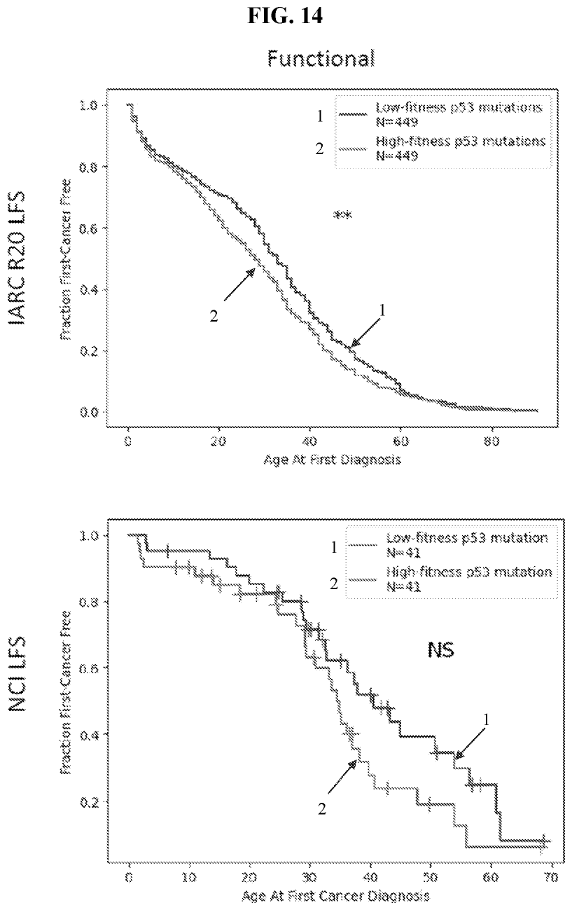

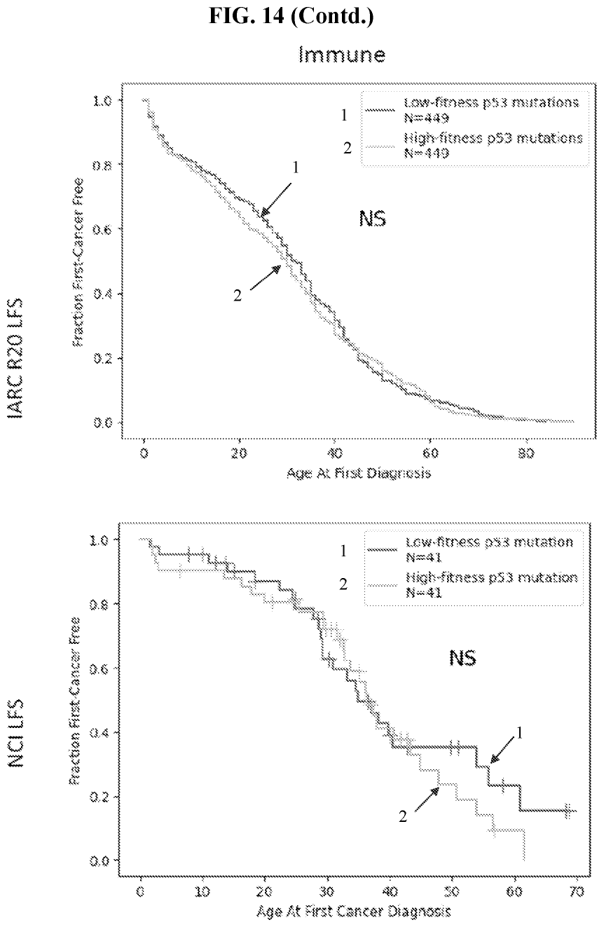

As germline TP53 mutations are the primary cause of Li-Fraumeni syndrome (LFS), a highly cancer-prone autosomal dominant disorder, it was hypothesized that mutant p53 fitness relates to time to first tumor formation in LFS patients. We plotted Kaplan-Meier curves showing age of tumor onset for persons with germline p53 mutations in the IARC R20 germline dataset as well as an independent LFS cohort coordinated by the National Cancer Institute (NCI), stratified based on p53 fitness using matched p53 mutation and HLA-I information available in the NCI cohort. As the IARC R20 germline dataset did not have matched HLA information, we inferred fitness using the TCGA haplotype population (Methods). We found functional and immune components were required for significant separation of time to onset (FIG. 13A), with the immune component required for significant separation across a range of relative weights (FIGS. 4A-4B; FIG. 13B, FIG. 14). These results may seem counterintuitive in that mutant p53 may be interpreted as “self” by the adaptive immune system in LFS patients. However, increased mutant p53 abundance, as well as loss of the wild-type TP53 allele, compounded by additional somatic mutations, may dramatically increase tumor immune surveillance and mutant p53 antigenicity during tumorigenesis. These findings suggest there may exist a role for immune surveillance and a potential for immune intervention in germline TP53-mutated tumors.

Finally, evidence is growing many non-cancerous cells in diverse tissues harbor somatic TP53 mutations that confer a competitive advantage, predisposing the clones containing such mutations to develop into cancerous lesions. To better characterize somatic TP53 mutations in non-cancerous cells, we collated mutation data from multiple published works across many mutated tissues. We found the same somatic hotspots in non-neoplastic cells (FIG. 4C). However, surprisingly, the frequency of hotspots changed: R175H is dramatically under-represented in non-neoplastic cells as compared to tumors (p-value <0.0001, two-sided binomial test), while the potentially more immunogenic R248Q/W mutations were among the most frequent mutations. The addition of an immune component improved predictions much less than in the neoplastic setting (FIG. 4F, FIG. 26), supporting the hypothesis that the change in hotspot frequencies between non-cancerous and cancerous datasets is driven by a hotspot mutation's immune fitness, particularly for hotspots generated by CpG mutations (FIG. 4D). We split the non-neoplastic TP53 mutation dataset into the largest tissue-specific subgroups and found that the immune weight depended on the tissue type, although it was always weaker than the typical value for tumors in TCGA (FIGS. 4E-4F). Overall, these findings suggest that in pre-cancerous lesions more transcriptionally-active oncogenic mutations likely predominate, possibly by offering selective replicative advantage to pre-neoplastic cells. However, for cancer to form, the necessity for immune escape becomes more critical; thus, more immunologically fit mutations may become more prominent.

Disclosed herein is a general mathematical framework for predicting the fitness of tumor driver mutations, utilizing a minimal biophysical model that integrates background mutation rate, protein concentration, functional fitness advantage, and immune fitness cost (code available). Hotspots are predicted to fall on a near-optimal Pareto front, where trade-offs often constrain immunogenic driver-gene neoantigens from being fully eliminated. Driver mutations may therefore remain targetable by immunotherapies. The R175H mutation retains the largest degree of wild-type functional activity among TP53 hotspots while having the greatest immune cost. By contrast, R248Q/W mutants, despite a very strong loss of transactivation phenotype, appear in tumors less frequently than R175H mutations. Our model offers an explanation by showing the R248 locus may create neoantigens more likely to be recognized by T cells as compared to R175H and, as such, the R248Q/W mutations are more likely to be immunoedited or evade the immune system through regulation. We successfully applied similar models to over two dozen commonly mutated cancer driver genes, particularly for PTEN and KRAS. Such trade-offs appear to play out in the transition to cancer: in non-cancerous TP53-mutated cells the frequency order of hotspot mutations changes and R248Q/W mutations become the most frequent, suggesting immunogenicity is less relevant, consistent with recent observations that immune editing is less relevant in non-cancerous mutations. Such insight helps define a window of opportunity for prophylactic immune intervention against mutant p53, such as vaccination. Additionally, our model shows both p53 functional and immune mutant fitness may play a role in determining the age of tumor onset in LFS, suggesting a potential benefit of targeting germline TP53 mutations immunotherapeutically. Finally, our free fitness framework lends itself naturally to interpretable, free-energy based machine learning methods.

### REFERENCES

1. Martinez-Jimenez, F. et al. A compendium of mutational cancer driver genes. Nature Reviews Cancer 20, 555-572 (2020).
2. Baugh, E. H., Ke, H., Levine, A. J., Bonneau, R. A. & Chan, C. S. Why are there hotspot mutations in the TP53 gene in human cancers? Cell Death & Differentiation 25, 154-160 (2018).
3. Petitjean, A. et al. Impact of mutant p53 functional properties on TP53 mutation patterns and tumor phenotype: lessons from recent developments in the IARC TP53 database. Human Mutation 28, 622-629 (2007).
4. Giacomelli, A. O. et al. Mutational processes shape the landscape of TP53 mutations in human cancer. Nature Genetics 50, 1381-1387 (2018).
5. Kato, S. et al. Understanding the function-structure and function-mutation relationships of p53 tumor suppressor protein by high-resolution missense mutation analysis. Proceedings of the National Academy of Sciences 100, 8424-8429 (2003).
6. Kotler, E. et al. A systematic p53 mutation library links differential functional impact to cancer mutation pattern and evolutionary conservation. Molecular Cell 71, 178-190 (2018).
7. Marty, R. et al. MHC-I genotype restricts the oncogenic mutational landscape. Cell 171, 1272-1283 (2017).
8. Pyke, R. M. et al. Evolutionary pressure against MHC class II binding cancer mutations. Cell 175, 416-428 (2018).
9. Ding, J. et al. Systematic analysis of somatic mutations impacting gene expression in 12 tumour types. Nature Communications 6, 1-13 (2015).
10. Huang, N., Shah, P. K. & Li, C. Lessons from a decade of integrating cancer copy number alterations with gene expression profiles. Briefings in Bioinformatics 13, 305-316 (2012).
11. Fehrmann, R. S. et al. Gene expression analysis identifies global gene dosage sensitivity in cancer. Nature Genetics 47, 115-125 (2015).
12. Köbel, M. et al. Optimized p53 immunohistochemistry is an accurate predictor of TP53 mutation in ovarian carcinoma. The Journal of Pathology: Clinical Research 2, 247-258 (2016).
13. Murnyák, B. & Hortobágyi, T. Immunohistochemical correlates of TP53 somatic mutations in cancer. Oncotarget 7, 64910 (2016).
14. Cole, A. J. et al. Assessing mutant p53 in primary high-grade serous ovarian cancer using immunohistochemistry and massively parallel sequencing. Scientific Reports 6, 1-12 (2016).
15. Tran E. et al. T-cell transfer therapy targeting mutant KRAS in cancer. New England Journal of Medicine 375, 2255-2262 (2016).
16. Hsiue E. H. et al. Targeting a neoantigen derived from a common TP53 mutation. Science 371, 6533 (2021).
17. Shamalov, K., Levy, S. N., Horovitz-Fried, M. & Cohen, C. J. The mutational status of p53 can influence its recognition by human T-cells. Oncoimmunology 6, e1285990 (2017).
18. Eigen, M. Selforganization of matter and the evolution of biological macromolecules. Naturwissenschaften 58, 465-523 (1971).
19. Gerland, U. & Hwa, T. On the selection and evolution of regulatory DNA motifs. Journal of Molecular Evolution 55, 386-400 (2002).
20. Łuksza, M. & Lässig, M. A predictive fitness model for influenza. Nature 507, 57-61 (2014).
21. Balachandran, V. P. et al. Identification of unique neoantigen qualities in long-term survivors of pancreatic cancer. Nature 551, 512-516 (2017).
22. Łuksza, M. et al. A neoantigen fitness model predicts tumour response to checkpoint blockade immunotherapy. Nature 551, 517-520 (2017).
23. Ma, L. et al. A plausible model for the digital response of p53 to DNA damage. Proceedings of the National Academy of Sciences 102, 14266-14271 (2005).
24. Gaglia, G., Guan, Y., Shah, J. V. & Lahav, G. Activation and control of p53 tetramerization in individual living cells. Proceedings of the National Academy of Sciences 110, 15497-15501(2013).
25. Hunter, J. C. et al. Biochemical and structural analysis of common cancer-associated KRAS mutations. Molecular Cancer Research 13, 1325-1335 (2015).
26. Shoval, O. et al. Evolutionary trade-offs, Pareto optimality, and the geometry of phenotype space. Science 336, 1157-1160 (2012).
27. Pinheiro, F., Warsi, O., Andersson, D. I., & Lässig, M. Metabolic fitness landscapes predict the evolution of antibiotic resistance. Nature Ecology & Evolution 5, 677-687 (2021).
28. Malekzadeh P. et al. Neoantigen screening identifies broad TP53 mutant immunogenicity in patients with epithelial cancers. The Journal of Clinical Investigation 129, 1109-1114 (2019).
29. Kratz, C. P. et al. Analysis of the Li-Fraumeni Spectrum Based on an International Germline TP53 Variant Data Set: An International Agency for Research on Cancer TP53 Database Analysis. JAMA Oncology 28, e214398 (2021).
30. De Andrade, K. C. et al. Cancer incidence, patterns, and genotype-phenotype associations in individuals with pathogenic or likely pathogenic germline TP53 variants: an observational cohort study. Lancet Oncology 22, 1787-1798 (2021).
31. Martincorena, I. & Campbell, P. J. Somatic mutation in cancer and normal cells. Science 349, 1483-1489 (2015).
32. Caushi, J. X. et al. Transcriptional programs of neoantigen-specific TIL in anti-PD-1-treated lung cancers. Nature 596, 126-132 (2021).
33. Bear, A. S. et al. Biochemical and functional characterization of mutant KRAS epitopes validates this oncoprotein for immunological targeting. Nature Communications 12, 1-6 (2021).
34. Malekzadeh, P. et al. Antigen experienced T cells from peripheral blood recognize p53 neoantigens. Clinical Cancer Research. 26, 1267-1276 (2020).
35. Colom, B. et al. Mutant clones in normal epithelium outcompete and eliminate emerging tumours. Nature 598, 510-514 (2021).
36. Levine, A. J., Ting, D. T. & Greenbaum, B. D. P53 and the defenses against genome instability caused by transposons and repetitive elements. Bioessays 38, 508-513 (2016).
37. Dayan, P., Hinton, G. E., Neal, R. M., & Zemel, R. S. The Helmholtz machine. Neural Computation 7, 889-904 (1995).
38. Lunter, G. & Hein, J. A nucleotide substitution model with nearest-neighbour interactions. Bioinformatics 20, i216-i223 (2004). URL https://doi.org/10.1093/bioinformatics/bth901.
39. Wheeler, D. L. et al. Database resources of the national center for biotechnology information. Nucleic Acids Research 36, D13-D21 (2007).
40. Cingolani, P. et al. A program for annotating and predicting the effects of single nucleotide poly-morphisms, SnpEff: SNPs in the genome of Drosophila melanogaster strain w¹¹¹⁸; iso-2; iso-3. Fly 6, 80-92 (2012).
41. Gaglia, G., Guan, Y., Shah, J. V. & Lahav, G. Activation and control of p53 tetramerization in individual living cells. Proceedings of the National Academy of Sciences 110, 15497-15501 (2013).
42. Weinberg, R. L., Veprintsev, D. B. & Fersht, A. R. Cooperative binding of tetrameric p53 to DNA. Journal of Molecular Biology 341, 1145-1159 (2004).
43. Weinberg, R. L., Veprintsev, D. B., Bycroft, M. & Fersht, A. R. Comparative binding of p53 to its promoter and DNA recognition elements. Journal of Molecular Biology 348, 589-596 (2005).
44. Weinberg, R. L., Freund, S. M., Veprintsev, D. B., Bycroft, M. & Fersht, A. R. Regulation of DNA binding of p53 by its C-terminal domain. Journal of Molecular Biology 342, 801-811 (2004).
45. He, F. et al. Interaction between p53 N terminus and core domain regulates specific and nonspecific DNA binding. Proceedings of the National Academy of Sciences 116, 8859-8868 (2019).
46. Cain, C., Miller, S., Ahn, J. & Prives, C. The N terminus of p53 regulates its dissociation from DNA. Journal of Biological Chemistry 275, 39944-39953 (2000).
47. Friedler, A., Veprintsev, D. B., Freund, S. M., Karoly, I. & Fersht, A. R. Modulation of binding of DNA to the C-terminal domain of p53 by acetylation. Structure 13, 629-636 (2005).
48. Jordan, J. J. et al. Low-level p53 expression changes transactivation rules and reveals superactivating sequences. Proceedings of the National Academy of Sciences 109, 14387-14392 (2012).
49. Bouaoun, L. et al. TP53 variations in human cancers: new lessons from the IARC TP53 database and genomics data. Human Mutation 37, 865-876 (2016).
50. Ma, L. et al. A plausible model for the digital response of p53 to DNA damage. Proceedings of the National Academy of Sciences 102, 14266-14271 (2005).
51. Li, J. et al. Explore, visualize, and analyze functional cancer proteomic data using the cancer proteome atlas. Cancer Research 77, e51-e54 (2017).
52. Li, J. et al. TCPA: a resource for cancer functional proteomics data. Nature Methods 10, 1046-1047 (2013).
53. Raine, K. M. et al. ascatNgs: Identifying somatically acquired copy-number alterations from whole-genome sequencing data. Current Protocols in Bioinformatics 56, 15-9 (2016).
54. Tate, J. G. et al. COSMIC: the catalogue of somatic mutations in cancer. Nucleic Acids Research 47, D941-D947 (2019).
55. Grossman, R. L. et al. Toward a shared vision for cancer genomic data. New England Journal of Medicine 375, 1109-1112 (2016).
56. Landau, D. A., Carter, S. L., Getz, G. & Wu, C. J. Clonal evolution in hematological malignancies and therapeutic implications. Leukemia 28, 34-43 (2014).
57. Dentro, S. C., Wedge, D. C. & Van Loo, P. Principles of reconstructing the subclonal architecture of cancers. Cold Spring Harbor Perspectives in Medicine 7, a026625 (2017).
58. Khiabanian, H. et al. Inference of germline mutational status and evaluation of loss of heterozygosity in high-depth, tumor-only sequencing data. JCO Precision Oncology 2, 1-15 (2018).
59. McGranahan, N. et al. Clonal status of actionable driver events and the timing of mutational processes in cancer evolution. Science Translational Medicine 7, 283ra54-283ra54 (2015).
60. Chan, W. M., Siu, W. Y., Lau, A. & Poon, R. Y. How many mutant p53 molecules are needed to inactivate a tetramer? Molecular and Cellular Biology 24, 3536-3551 (2004).
61. Lundegaard, C., Lund, O. & Nielsen, M. Accurate approximation method for prediction of class I MHC affinities for peptides of length 8, 10 and 11 using prediction tools trained on 9mers. Bioinformatics 24, 1397-1398 (2008).
62. Consortium, T. U. UniProt: The universal protein knowledgebase in 2021. Nucleic Acids Research 49, D480-D489 (2021).
63. Nielsen, M. et al. Reliable prediction of T-cell epitopes using neural networks with novel sequence representations. Protein Science 12, 1007-1017 (2003).
64. Lundegaard, C. et al. NetMHC-3.0: accurate web accessible predictions of human, mouse and monkey MHC class I affinities for peptides of length 8-11. Nucleic Acids Research 36, W509-W512 (2008).
65. Andreatta, M. & Nielsen, M. Gapped sequence alignment using artificial neural networks: application to the MHC class I system. Bioinformatics 32, 511-517 (2016).
66. Thorsson, V. et al. The immune landscape of cancer. Immunity 48, 812-830 (2018).
67. Gonzalez-Galarza, F. F. et al. Allele frequency net database (AFND) 2020 update: gold-standard data classification, open access genotype data and new query tools. Nucleic Acids Research 48, D783-D788 (2020).
68. Gragert, L., Madbouly, A., Freeman, J. & Maiers, M. Six-locus high resolution HLA haplotype frequencies derived from mixed-resolution DNA typing for the entire US donor registry. Human Immunology 74, 1313-1320 (2013).
69. Ashkenazy, H. et al. ConSurf 2016: an improved methodology to estimate and visualize evolutionary conservation in macromolecules. Nucleic Acids Research 44, W344-W350 (2016).
70. Berezin, C. et al. ConSeq: the identification of functionally and structurally important residues in protein sequences. Bioinformatics 20, 1322-1324 (2004).
71. Suzek, B. E. et al. UniRef clusters: a comprehensive and scalable alternative for improving sequence similarity searches. Bioinformatics 31, 926-932 (2015).
72. Li, W. & Godzik, A. Cd-hit: a fast program for clustering and comparing large sets of protein or nucleotide sequences. Bioinformatics 22, 1658-1659 (2006).
73. Fu, L., Niu, B., Zhu, Z., Wu, S. & Li, W. CD-HIT: accelerated for clustering the next-generation sequencing data. Bioinformatics 28, 3150-3152 (2012).
74. Katoh, K. & Standley, D. M. MAFFT multiple sequence alignment software version 7: improvements in performance and usability. Molecular Biology and Evolution 30, 772-780 (2013).
75. Pupko, T., Bell, R. E., Mayrose, I., Glaser, F. & Ben-Tal, N. Rate4Site: an algorithmic tool for the identification of functional regions in proteins by surface mapping of evolutionary determinants within their homologues. Bioinformatics 18, S71-S77 (2002).
76. Bullock, A. N., Henckel, J. & Fersht, A. R. Quantitative analysis of residual folding and DNA binding in mutant p53 core domain: definition of mutant states for rescue in cancer therapy. Oncogene 19, 1245-1256 (2000).
77. Tan, Y. & Luo, R. Structural and functional implications of p53 missense cancer mutations. PMC Biophysics 2,5 (2009).
78. Chen, Y., Dey, R. & Chen, L. Crystal structure of the p53 core domain bound to a full consensus site as a self-assembled tetramer. Structure 18, 246-256 (2010).
79. Mageean, C. J., Griffiths, J. R., Smith, D. L., Clague, M. J. & Prior, I. A. Absolute quantification of endogenous Ras isoform abundance. PLoS One 10, e0142674 (2015).
80. Corces, M. R. et al. The chromatin accessibility landscape of primary human cancers. Science 362 (2018).
81. Baek, S., Goldstein, I. & Hager, G. L. Bivariate genomic footprinting detects changes in transcription factor activity. Cell Reports 19, 1710-1722 (2017).
82. Mai, P. L. et al. Risks of first and subsequent cancers among TP53 mutation carriers in the National Cancer Institute Li-Fraumeni syndrome cohort. Cancer 122, 3673-3681 (2016).
83. Zheng, X. et al. HIBAG-HLA genotype imputation with attribute bagging. The Pharmacogenomics Journal 14, 192-200 (2014).
84. Klinger, M. et al. Multiplex identification of antigen-specific T cell receptors using a combination of immune assays and immune receptor sequencing. PLoS One 10, e0141561 (2015).
85. Jurtz, V. et al. NetMHCpan-4.0: improved peptide-MHC class I interaction predictions integrating eluted ligand and peptide binding affinity data. The Journal of Immunology 199, 3360-3368 (2017).
86. Snyder, T. M. et al. Magnitude and dynamics of the T-cell response to SARS-CoV-2 infection at both individual and population levels. MedRxiv (2020).
87. Grossman, R. L. et al. Toward a shared vision for cancer genomic data. New England Journal of Medicine 375, 1109-1112 (2016).
88. Thom, C. S., Dickson, C. F., Gell, D. A. & Weiss, M. J. Hemoglobin variants: biochemical properties and clinical correlates. Cold Spring Harbor Perspectives in Medicine 3, a011858 (2013).
89. Kaufman, D. P., Khattar, J. & Lappin, S. L. Physiology, Fetal Hemoglobin. StatPearls [Internet](2021).
90. Scriver, C. R. The PAH gene, phenylketonuria, and a paradigm shift. Human Mutation 28, 831-845 (2007).
91. Oldenburg, J. & El-Maarri, O. New insight into the molecular basis of hemophilia A. International Journal of Hematology 83, 96-102 (2006).
92. Dixon, P. H. et al. Mutational analysis of PHEX gene in X-linked hypophosphatemia. The Journal of Clinical Endocrinology & Metabolism 83, 3615-3623 (1998).
93. Assia Batzir, N. et al. Phenotypic expansion of POGZ-related intellectual disability syndrome (White-Sutton syndrome). American Journal of Medical Genetics Part A 182, 38-52 (2020).
94. Sherry, S. T. et al. dbSNP: the NCBI database of genetic variation. Nucleic Acids Research 29, 308-311 (2001).
95. Landrum, M. J. et al. ClinVar: improving access to variant interpretations and supporting evidence. Nucleic Acids Research 46, D1062-D1067 (2018).
96. Tate, J. G. et al. COSMIC: the catalogue of somatic mutations in cancer. Nucleic Acids Research 47, D941-D947 (2019).
97. Consortium, T. U. UniProt: The universal protein knowledgebase in 2021. Nucleic Acids Research 49, D480-D489 (2021).
98. Abelson, S. et al. Prediction of acute myeloid leukaemia risk in healthy individuals. Nature 559, 400-404 (2018).
99. Desai, P. et al. Somatic mutations precede acute myeloid leukemia years before diagnosis. Nature Medicine 24, 1015-1023 (2018).
100. Wong, T. N. et al. Role of TP53 mutations in the origin and evolution of therapy-related acute myeloid leukaemia. Nature 518, 552-555 (2015).
101. Jaiswal, S. et al. Age-related clonal hematopoiesis associated with adverse outcomes. New England Journal of Medicine 371, 2488-2498 (2014).
102. Genovese, G. et al. Clonal hematopoiesis and blood-cancer risk inferred from blood DNA sequence. New England Journal of Medicine 371, 2477-2487 (2014).
103. Xie, M. et al. Age-related mutations associated with clonal hematopoietic expansion and malignancies. Nature Medicine 20, 1472-1478 (2014).
104. Li, R. et al. Macroscopic somatic clonal expansion in morphologically normal human urothelium. Science 370, 82-89 (2020).
105. Lawson, A. R. et al. Extensive heterogeneity in somatic mutation and selection in the human bladder. Science 370, 75-82 (2020).
106. Yoshida, K. et al. Tobacco smoking and somatic mutations in human bronchial epithelium. Nature 578, 266-272 (2020).
107. Franklin, W. A. et al. Widely dispersed p53 mutation in respiratory epithelium. a novel mechanism for field carcinogenesis. The Journal of Clinical Investigation 100, 2133-2137 (1997).
108. Kadara, H. et al. Driver mutations in normal airway epithelium elucidate spatiotemporal resolution of lung cancer. American Journal of Respiratory and Critical Care Medicine 200, 742-750 (2019).
109. Lee-Six, H. et al. The landscape of somatic mutation in normal colorectal epithelial cells. Nature 574, 532-537 (2019).
110. Olafsson, S. et al. Somatic evolution in non-neoplastic IBD-affected colon. Cell 182, 672-684 (2020).
111. Matas, J. et al. Colorectal cancer is associated with the presence of cancer driver mutations in normal colon. medRxiv (2021).
112. Salk, J. J. et al. Ultra-sensitive TP53 sequencing for cancer detection reveals progressive clonal selection in normal tissue over a century of human lifespan. Cell Reports 28, 132-144 (2019).
113. Krimmel, J. D. et al. Ultra-deep sequencing detects ovarian cancer cells in peritoneal fluid and reveals somatic TP53 mutations in noncancerous tissues. Proceedings of the National Academy of Sciences 113, 6005-6010 (2016).
114. Krimmel-Morrison, J. D. et al. Characterization of TP53 mutations in Pap test DNA of women with and without serous ovarian carcinoma. Gynecologic Oncology 156, 407-414 (2020).
115. Paracchini, L. et al. Detection of TP53 clonal variants in Papanicolaou test samples collected up to 6 years prior to high-grade serous epithelial ovarian cancer diagnosis. JAMA Network Open 3, e207566-e207566 (2020).
116. Jia, L. et al. Endometrial glandular dysplasia with frequent p53 gene mutation: a genetic evidence supporting its precancer nature for endometrial serous carcinoma. Clinical Cancer Research 14, 2263-2269 (2008).
117. Moore, L. et al. The mutational landscape of normal human endometrial epithelium. Nature 580, 640-646 (2020).
118. Anglesio, M. S. et al. Cancer-associated mutations in endometriosis without cancer. New England Journal of Medicine 376, 1835-1848 (2017).
119. Martincorena, I. et al. Somatic mutant clones colonize the human esophagus with age. Science 362, 911-917 (2018).
120. Yokoyama, A. et al. Age-related remodelling of oesophageal epithelia by mutated cancer drivers. Nature 565, 312-317 (2019).
121. Prevo, L. J., Sanchez, C. A., Galipeau, P. C. & Reid, B. J. p53-mutant clones and field effects in Barrett's esophagus. Cancer Research 59, 4784-4787 (1999).
122. Mandard, A.-M., Hainaut, P. & Hollstein, M. Genetic steps in the development of squamous cell carcinoma of the esophagus. Mutation Research/Reviews in Mutation Research 462, 335-342 (2000).
123. Weaver, J. M. et al. Ordering of mutations in preinvasive disease stages of esophageal carcinogenesis. Nature Genetics 46, 837-843 (2014).
124. Waridel, F. et al. Field cancerisation and polyclonal p53 mutation in the upper aero-digestive tract. Oncogene 14, 163-169 (1997).
125. Ross-Innes, C. S. et al. Whole-genome sequencing provides new insights into the clonal architecture of Barrett's esophagus and esophageal adenocarcinoma. Nature Genetics 47, 1038-1046 (2015).
126. Stachler, M. D. et al. Paired exome analysis of Barrett's esophagus and adenocarcinoma. Nature Genetics 47, 1047-1055 (2015).
127. Yuan, W. et al. Clonal evolution of esophageal squamous cell carcinoma from normal mucosa to primary tumor and metastases. Carcinogenesis 40, 1445-1451 (2019).
128. Kim, S. K. et al. Comprehensive analysis of genetic aberrations linked to tumorigenesis in regenerative nodules of liver cirrhosis. Journal of Gastroenterology 54, 628-640 (2019).
129. Ling, G. et al. Persistent p53 mutations in single cells from normal human skin. The American Journal ofPathology 159, 1247-1253 (2001).
130. Martincorena, I. et al. High burden and pervasive positive selection of somatic mutations in normal human skin. Science 348, 880-886 (2015).
131. Jonason, A. S. et al. Frequent clones of p53-mutated keratinocytes in normal human skin. Proceedings of the National Academy of Sciences 93, 14025-14029 (1996).
132. Simons, B. D. Deep sequencing as a probe of normal stem cell fate and preneoplasia in human epidermis. Proceedings of the National Academy of Sciences 113, 128-133 (2016).
133. Ren, Z.-P. et al. Benign clonal keratinocyte patches with p53 mutations show no genetic link to synchronous squamous cell precancer or cancer in human skin. The American Journal of Pathology 150, 1791 (1997).
134. Bäckvall, H. et al. Mutation spectra of epidermal p53 clones adjacent to basal cell carcinoma and squamous cell carcinoma. Experimental Dermatology 13, 643-650 (2004).
135. Hernando, B. et al. The effect of age on the acquisition and selection of cancer driver mutations in sun-exposed normal skin. Annals of Oncology 32, 412-421 (2021).
136. Tang, J. et al. The genomic landscapes of individual melanocytes from human skin. Nature 586, 600-605 (2020).
137. Muradova, E. et al. Noninvasive assessment of epidermal genomic markers of UV exposure in skin. Journal of Investigative Dermatology 141, 124-131 (2021).
138. Coorens, T. H. et al. Extensive phylogenies of human development inferred from somatic mutations. Nature 597, 387-392 (2021).
139. Moore, L. et al. The mutational landscape of human somatic and germline cells. Nature 597, 381-386 (2021).
140. Xia, L. et al. Statistical analysis of mutant allele frequency level of circulating cell-free DNA and blood cells in healthy individuals. Scientific Reports 7, 1-7 (2017).
141. Yizhak, K. et al. RNA sequence analysis reveals macroscopic somatic clonal expansion across normal tissues. Science 364 (2019).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for selecting a candidate therapy based on mutant p53 fitness, comprising:
(a) obtaining, by one or more processors of a computing device, a dataset comprising a plurality of p53 missense mutations present in one or more subjects;
(b) for each p53 missense mutation in the dataset, applying a multi-parameter orthogonal model to obtain a fitness score, wherein the multi-parameter orthogonal model comprises:
(i) generating, by the one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene;
(ii) generating, by the one or more processors, an immunogenic cost metric for the p53 missense mutation based on binding affinities of MHC class I molecules to p53-derived nonamer neopeptides including the p53 missense mutation; and
(iii) generating, by the one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric;
(c) identifying, by the one or more processors, a subset of p53 missense mutations that have fitness scores that exceed a threshold;
(d) selecting, by the one or more processors, adoptive T-cell therapy or neoantigen vaccine therapy for the subset of p53 missense mutations; and
(e) storing, by the one or more processors, in a computer-readable non-volatile memory device, adoptive T-cell therapy or neoantigen vaccine therapy in association with the subset of p53 missense mutations as a candidate therapy,
optionally wherein
the pro-oncogenic advantage metric is assigned a greater weight relative to the immunogenic cost metric; or
the at least one p53 target gene is WAF1, MDM2, BAX, h1433s, AIP1, GADD45, NOXA, or P53R2; or
the transactivation levels of the at least one p53 target gene are determined using quantitative transactivation assays in yeast; or
the pro-oncogenic advantage metric is a median probability of the p53 polypeptide encoded by the p53 missense mutation not binding to the promoter region of the at least one p53 target gene, optionally wherein generating the pro-oncogenic advantage metric comprises applying, by the one or more processors, a cooperative Hill function; or
the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric; or
the divergence-based statistical analysis comprises minimizing, by the one or more processors, divergence scores between observed and predicted frequencies of the p53 missense mutation, optionally wherein the divergence scores that are minimized are Kullback-Leibler divergences; or
the dataset is generated, by the one or more processors, from DNA sequencing data obtained from one or more patients diagnosed with or at risk for cancer or Li-Fraumeni syndrome (LFS), optionally wherein the cancer is colorectal cancer, lung cancer, breast cancer, ovarian cancer, uterine cancer, or thyroid cancer; or
the MHC class I molecules comprise HLA-A alleles, HLA-B alleles, and HLA-C alleles; or
generating the immunogenic cost metric comprises determining, by the one or more processors, a geometric mean of probabilities of the p53-derived nonamer neopeptides including the p53 missense mutation binding each allele of the MHC class I molecules; or
the MHC class I molecules comprise one or more HLA alleles selected from the group consisting of A*02:11, A*26:02, A*68:23, C*07:01, A*02:03, A*02:06, C*12:03, A*68:02, A*24:03, B*15:03, B*15:17, B*57:01, B*58:01, A*31:01, A*33:01, A*68:01, A*11:01, A*30:01, A*32:07, B*08:01, C*03:03, A*02:01, A*02:12, A*02:17, B*39:01, and B*73:01; or
the plurality of p53 missense mutations comprises somatic and/or germline p53 mutations.

2. (canceled)

3. (canceled)

4. The method of claim 1, further comprising administering the adoptive T-cell therapy or neoantigen vaccine therapy to a patient comprising at least one p53 missense mutation that is present in the subset of p53 missense mutations, optionally wherein the neoantigen vaccine therapy is a RNA neoantigen vaccine, a synthetic long peptide neoantigen vaccine, or a dendritic cell (DC)-based neoantigen vaccine.

5. (canceled)

6. (canceled)

7. (canceled)

8. (canceled)

9. A method for selecting a candidate anti-cancer therapy based on mutant p53 fitness, comprising:
(a) obtaining, by one or more processors of a computing device, a dataset comprising a plurality of p53 missense mutations present in one or more subjects;
(b) for each p53 missense mutation in the dataset, applying a model to obtain a fitness score, wherein the model comprises:
(i) generating, by the one or more processors, an immunogenic cost metric for the p53 missense mutation based on binding affinities of MHC class I molecules to p53-derived nonamer neopeptides including the p53 missense mutation; and
(ii) generating, by the one or more processors, based on the immunogenic cost metric, a fitness score, wherein generating the fitness score comprises applying a divergence-based statistical analysis to optimize the immunogenic cost metric;
(c) identifying a subset of p53 missense mutations that have fitness scores that fall below a threshold;
(d) selecting, by the one or more processors, an immune checkpoint blockade therapy for the subset of p53 missense mutations; and
(e) storing, by the one or more processors, in a computer-readable non-volatile memory device, the immune checkpoint blockade therapy in association with the subset of p53 missense mutations as a candidate anti-cancer therapy,
optionally wherein
the multi-parameter orthogonal model further comprises: generating, by the one or more processors, a logarithmic frequency metric for the p53 missense mutation based on background frequency of the p53 missense mutation, and generating by the one or more processors, based on the pro-oncogenic advantage metric, the immunogenic cost metric, and the logarithmic frequency metric, a free fitness score, wherein generating the free fitness score comprises aggregating the pro-oncogenic advantage metric, the immunogenic cost metric, and the logarithmic frequency metric; or
the divergence-based statistical analysis comprises minimizing, by the one or more processors, divergence scores between observed and predicted frequencies of the p53 missense mutation, optionally wherein the divergence scores that are minimized are Kullback-Leibler divergences; or
the dataset is generated, by the one or more processors, from DNA sequencing data obtained from one or more patients diagnosed with or at risk for cancer, optionally wherein the cancer is colorectal cancer, lung cancer, breast cancer, ovarian cancer, uterine cancer, or thyroid cancer; or
the MHC class I molecules comprise HLA-A alleles, HLA-B alleles, and HLA-C alleles; or
generating the immunogenic cost metric comprises determining, by the one or more processors, a geometric mean of probabilities of the p53-derived nonamer neopeptides including the p53 missense mutation binding each allele of the MHC class I molecules; or
the MHC class I molecules comprise one or more HLA alleles selected from the group consisting of A*02:11, A*26:02, A*68:23, C*07:01, A*02:03, A*02:06, C*12:03, A*68:02, A*24:03, B*15:03, B*15:17, B*57:01, B*58:01, A*31:01, A*33:01, A*68:01, A*11:01, A*30:01, A*32:07, B*08:01, C*03:03, A*02:01, A*02:12, A*02:17, B*39:01, and B*73:01; or
the plurality of p53 missense mutations comprises somatic and/or germline p53 mutations.

10. (canceled)

11. The method of claim 9, further comprising administering the immune checkpoint blockade therapy to a cancer patient comprising at least one p53 missense mutation that is present in the subset of p53 missense mutations, optionally wherein the immune checkpoint blockade therapy comprises anti-PD-L1 therapy, anti-PD-1 therapy, or anti-CTLA4 therapy.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A method for selecting a patient diagnosed with or at risk for cancer for treatment with an immune checkpoint inhibitor comprising:
detecting the presence of a p53 mutation in a biological sample obtained from the patient, wherein the p53 mutation is selected from the group consisting of R248Q, R273H, R248W, R273C, and G245S; and
administering to the patient an effective amount of the immune checkpoint inhibitor.

24. The method of claim 23, wherein the immune checkpoint inhibitor is an anti-PD-L1 therapy, an anti-PD-1 therapy, or an anti-CTLA4 therapy.

25. The method of claim 23, wherein the cancer is colorectal cancer, lung cancer, breast cancer, ovarian cancer, uterine cancer, or thyroid cancer.

26. The method of claim 23, wherein the biological sample comprises blood, plasma, serum or tissue.

27. The method of claim 23, wherein the p53 mutation is detected via in situ hybridization, polymerase chain reaction (PCR), Next-generation sequencing, Northern blotting, microarray, dot or slot blots, fluorescent in situ hybridization (FISH), electrophoresis, chromatography, or mass spectroscopy.

28. A method for classifying tumor behavior for a potential tumor based on mutant p53 fitness, comprising:
(a) obtaining, by one or more processors of a computing device, a dataset comprising a plurality of p53 missense mutations present in one or more subjects;
(b) for each p53 missense mutation in the dataset, applying a multi-parameter orthogonal model to obtain a fitness score, wherein the multi-parameter orthogonal model comprises:
(i) generating, by the one or more processors, a pro-oncogenic advantage metric for the p53 missense mutation based on a decrease in transactivation levels of at least one p53 target gene caused by reduced binding of a p53 polypeptide encoded by the p53 missense mutation to a promoter region of the at least one p53 target gene;
(ii) generating, by the one or more processors, an immunogenic cost metric for the p53 missense mutation based on binding affinities of MHC class I molecules to p53-derived nonamer neopeptides including the p53 missense mutation; and
(iii) generating, by the one or more processors, based on the pro-oncogenic advantage metric and the immunogenic cost metric, a fitness score, wherein generating the fitness score comprises assigning weights to the pro-oncogenic advantage metric and the immunogenic cost metric, and applying a divergence-based statistical analysis to optimize the pro-oncogenic advantage metric and the immunogenic cost metric;
(c) identifying, by the one or more processors, a subset of p53 missense mutations that have fitness scores that exceed a threshold;
(d) identifying, by the one or more processors, at least one of an age of tumor onset or a tumor type corresponding to the potential tumor for the subset of p53 missense mutations; and
(e) storing, by the one or more processors, in a computer-readable non-volatile memory device, the at least one of the age of tumor onset or the tumor type in association with the subset of p53 missense mutations as a tumor behavior classification.

29. The method of claim 28, wherein the tumor behavior classification identifies the age of tumor onset as 10-20 years.

30. The method of claim 28, wherein the tumor behavior classification identifies the age of tumor onset as 30-50 years

31. The method of claim 28, wherein the tumor behavior classification identifies the age of tumor onset as 50 years or older.

32. (canceled)

33. (canceled)

34. (canceled)

35. (canceled)

36. (canceled)

37. The method of claim 28, wherein the divergence-based statistical analysis comprises minimizing, by the one or more processors, divergence scores between observed and predicted frequencies of the p53 missense mutation, optionally wherein the divergence scores that are minimized are Kullback-Leibler divergences.

38. (canceled)

39. The method of claim 28, wherein the MHC class I molecules comprise HLA-A alleles, HLA-B alleles, and HLA-C alleles.

40. The method of claim 28, wherein generating the immunogenic cost metric comprises determining, by the one or more processors, a geometric mean of probabilities of the p53-derived nonamer neopeptides including the p53 missense mutation binding each allele of the MHC class I molecules.

41. The method of claim 28, wherein the MHC class I molecules comprise one or more HLA alleles selected from the group consisting of A*02:11, A*26:02, A*68:23, C*07:01, A*02:03, A*02:06, C*12:03, A*68:02, A*24:03, B*15:03, B*15:17, B*57:01, B*58:01, A*31:01, A*33:01, A*68:01, A*11:01, A*30:01, A*32:07, B*08:01, C*03:03, A*02:01, A*02:12, A*02:17, B*39:01, and B*73:01.

42. The method of claim 28, wherein the dataset is generated, by the one or more processors, from DNA sequencing data obtained from one or more patients diagnosed with or at risk for Li-Fraumeni syndrome (LFS).

43. The method of claim 28, wherein the tumor behavior classification identifies the tumor type as corresponding to colorectal cancer, lung cancer, breast cancer, ovarian cancer, uterine cancer, or thyroid cancer.

44. The method of claim 28, wherein the plurality of p53 missense mutations comprises germline p53 mutations.

45. (canceled)

46. (canceled)

47. (canceled)

48. (canceled)
